(19) United States
(12) Patent Application Publication
(10) Pub. No.: US 2026/0184769 A1
(43) Pub. Date: Jul. 2, 2026

(54) YEAST-BASED IMMUNOTHERAPY AGAINST CLOSTRIDIUM DIFFICILE INFECTION

(71) Applicants: Hanping Feng, Ellicott City, MD (US); James Eugene GALEN, Eldersburg, MD (US); Kevin CHEN, Baltimore, MD (US); Yixuan ZHU, Ellicott City, MD (US)

(72) Inventors: Hanping Feng, Ellicott City, MD (US); James Eugene GALEN, Eldersburg, MD (US); Kevin CHEN, Baltimore, MD (US); Yixuan ZHU, Ellicott City, MD (US)

(73) Assignee: University of Maryland, Baltimore, Baltimore, MD (US)

(21) Appl. No.: 19/275,250

(22) Filed: Jul. 21, 2025

Related U.S. Application Data
(63) Continuation of application No. 17/955,537, filed on Sep. 29, 2022, now Pat. No. 12,384,833.
Application No. 17/955,537 is a division of application No. 15/768,331, now Pat. No. 11,466,075.
(60) Provisional application No. 62/240,810, filed on Oct. 13, 2015.

(51) Int. Cl. C07K 16/1282; A61K 9/00; A61K 36/064; A61K 39/00; A61K 45/06; A61P 31/04; C12N 1/18; G01N 33/569

(52) U.S. Cl. CPC C07K 16/1282; A61K 9/0031; A61K 9/0043; A61K 9/0053; A61K 36/064; A61K 45/06; A61P 31/04; C12N 1/18; G01N 33/56961; A61K 2039/505; C07K 2317/14; C07K 2317/21; C07K 2317/22; C07K 2317/31; C07K 2317/35; C07K 2317/52; C07K 2317/569; C07K 2317/64; C07K 2317/76; C07K 2317/94

(57) ABSTRACT

Antibody-based binding agents derived from human and camelid immunoglobulins are described, as well as strains of yeast engineered to secrete the binding agents, and methods of treating and preventing Clostridium difficile infections using the engineered strains of yeast. These binding agents recognize and bind with specificity to Clostridium difficile toxin A and/or toxin B and in some cases exhibit toxin neutralizing activity. The binding agents include camelid V_{H}H peptide monomers, linked groups of V_{H}H peptide monomers, V_{H}H peptide monomers joined to antibody Fc domains, and V_{H}H peptide monomers joined to IgG antibodies.

### CROSS-REFERENCE TO RELATED APPLICATIONS

This application is a continuation of U.S. Non-Provisional application Ser. No. 17/955,537, filed on Sep. 29, 2022, now U.S. Pat. No. 12,384,833, which is a divisional of U.S. Non-Provisional application Ser. No. 15/768,331, filed on Apr. 13, 2018, now U.S. Pat. No. 11,466,075, which is a national stage of International Patent Application No. PCT/US2016/056875, filed on Oct. 13, 2016, which claims priority to U.S. Provisional Application No. 62/240,810, filed Oct. 13, 2015, the contents of each which are hereby incorporate by reference in their entirety.

### STATEMENT OF FEDERALLY SPONSORED RESEARCH AND DEVELOPMENT

This invention was made with government support under Grant Numbers DK084509 and AI109776 awarded by the National Institutes of Health. The government has certain rights in the invention.

### SEQUENCE LISTING

A sequence listing in electronic ST.26 XML format is filed with this application and incorporated herein by reference. The name of the XML file is “130507-0127_ST26_SL”; the file was created on Jan. 23, 2023; the size of the file is 387,826 bytes.

### BACKGROUND

The bacterium Clostridium difficile is the most common cause of nosocomial antibiotic-associated diarrhea as well as the etiologic agent of pseudomembranous colitis [1]. It is estimated that over 500,000 cases of C. difficile-associated disease (CDI) occur annually in the United States, with the annual mortality rate ranging from about 3-17%, depending on the strains. With the emergence of hypervirulent and antibiotic-resistant strains, the incidence of mortality in CDI patients is increasing rapidly [2].

CDI is mainly caused by the two C. difficile exotoxins TcdA and TcdB (as TedA-TcdB-strains are avirulent) [21,22]. The two toxins are structurally similar and exhibit a similar mode of action on host cells. Both toxins target host Rho GTPases, leading to their inactivation as well as cytoskeleton disorganization. The relative roles of the two toxins in the pathogenesis of CDI are not well understood, but it is clear that either toxin individually can cause CDI in animals [22,23].

The options for treating CDI patients are limited and the recurrence rate is high (20-35% of patients). Current standard treatment for CDI using antibiotics causes the disruption of microflora and results in a relapse rate approaching 35% [3,13]. While other interventions have been tried (e.g., probiotics, toxin-absorbing polymers, and toxoid vaccines), neither prevention nor treatment strategies have kept up with the increased incidence and severity of this infection. The risk of further episodes of CDI in recurrent patients can be more than 50% and a subset of patients will have multiple recurrences. Recurrent CDI can be caused by the same strain or newly colonizing strains [15-18].

Newer immune-based therapies have been shown to be somewhat effective in clinical trials, including intravenous immunoglobulin (IVIG) against severe CDI [4-8] and human monoclonal antibodies against recurrent CDI [9]. Fidaxomicin, a narrow spectrum macrocyclic antibiotic, showed an effect similar to oral vancomycin on CDI but was significantly better at lowering the relapse rate [10]. Fecal transplantation is effective against refractory and recurrent CDI, but it is difficult to standardize and it is associated with risks [11,12].

CDI is a frustrating condition that is difficult to treat and may affect patients for months or even years, causing tremendous morbidity and mortality [19]. Accordingly, there is a need for new treatments for CDI, and means for preventing both primary and recurrent CDI in subjects at risk of developing CDI.

### BRIEF SUMMARY OF INVENTION

Provided herein are antibody-based fusion protein binding agents that selectively bind C. difficile virulence factors TcdA and TcdB, and strains of the probiotic yeast Saccharomyces genetically engineered to express and secrete these C. difficile toxin binding agents. Both the yeast and the binding agents show utility in treating and preventing primary and recurrent CDI in a subject. Orally administered Saccharomyces secreting the binding agents in host intestines can relieve ongoing CDI and prevent recurrence.

The present invention is thus directed to C. difficile toxin binding agents, strains of Saccharomyces including, but not limited to, Saccharomyces boulardii engineered to produce the binding agents, methods of making the engineered strains of yeast, and methods of treating and preventing primary and recurrent CDI using the binding agents and the engineered strains of yeast, among other important features.

### Binding Agents

The binding agents of the present invention include simple V_{H}H peptide monomers and linked groups of V_{H}H peptide monomers (comprising 2, 3, 4, or more monomers), as well as more complex binding agents that comprise V_{H}H peptide monomers joined to antibody Fc domains, as well as V_{H}H peptide monomers joined to partial or full IgG antibodies.

In a first embodiment, the present invention is directed to binding agents comprising V_{H}H peptide monomers and linked groups of V_{H}H peptide monomers comprising two, three, four, or more monomers, each of which binds TcdA and/or TcdB, preferably with specificity. Thus, the invention encompasses V_{H}H peptide binding agents comprising at least one V_{H}H peptide monomer, wherein each V_{H}H peptide monomer has binding specificity for an epitope of C. difficile toxin A (TcdA) or toxin B (TcdB). In certain aspects, these binding agents comprise two, three, four, or more linked V_{H}H peptide monomers. The V_{H}H peptide monomers include, but are not limited to, the V_{H}H peptide monomers 5D (SEQ ID NO:1), E3 (SEQ ID NO:3), AA6 (SEQ ID NO:5), and AH3 (SEQ ID NO:7).

In aspects of this embodiment where two or more monomer are linked, the monomers may be linked by flexible peptide linkers, generally comprising between 10 and 20 amino acids. Suitable linkers include, but are not limited to, linker-1 (SEQ ID NO:9), linker-2 (SEQ ID NO: 11), and linker-3 (SEQ ID NO:13).

In certain aspects of this embodiment, the binding agents bind to TedA and/or TcdB with specificity. In certain aspects of this embodiment, the binding agents exhibit TcdA and/or TcdB neutralizing activity.

In a specific aspect of this embodiment, the binding agent comprises four linked V_{H}H peptide monomers where two of the monomers have binding specificity for epitopes of TcdA and two of the monomers have binding specificity for epitopes of TcdB. The epitopes of TcdA may be the same or different. The epitopes of TcdB may be the same or different.

In a specific aspect of this embodiment, the binding agent comprises the amino acid sequence set forth in SEQ ID NO:19 or a sequence variant thereof having at least 95% sequence identity thereto, and wherein the sequence variant retains TcdA and/or TcdB binding specificity, or the sequence variant retains toxin neutralizing activity, or both. In some instances, variant amino acids of the sequence variant are located in framework regions of the V_{H}H peptide monomers.

In a second embodiment, the invention is directed to binding agents comprising V_{H}H peptide monomers joined to IgG antibodies, where the binding agents bind TedA and/or TcdB. In these IgG-based binding agents, the variable regions of the light and heavy chains of IgG antibodies are replaced by one, two, three, four or more of the V_{H}H peptide monomers.

In certain aspects of this embodiment, these binding agents comprise two, three, four, or more linked V_{H}H peptide monomers joined to the amino termini of IgG light and heavy chains in place of the variable regions. The V_{H}H peptide monomers include, but are not limited to, the V_{H}H peptide monomers 5D (SEQ ID NO:1), E3 (SEQ ID NO:3), AA6 (SEQ ID NO:5), and AH3 (SEQ ID NO:7).

In aspects of this embodiment where two or more monomer are linked, the monomers may be linked by flexible peptide linkers, generally comprising between 10 and 20 amino acids. Suitable linkers include, but are not limited to, linker-1 (SEQ ID NO:9), linker-2 (SEQ ID NO: 11), and linker-3 (SEQ ID NO:13).

In a first sub-embodiment, the invention is directed to tetra-specific, octameric binding agents comprising an IgG antibody, two sets of linked first and second V_{H}H peptide monomers, and two sets of linked third and fourth V_{H}H peptide monomers, wherein the IgG antibody comprises two arms, each arm comprising a heavy chain lacking a variable region and a light chain lacking a variable region, and each chain having an amino terminus, wherein for each arm of the antibody, one set of linked first and second V_{H}H peptide monomers is joined to the amino terminus of the light chain, and one set of linked third and fourth V_{H}H peptide monomers is joined to the amino terminus of the heavy chain, and wherein the V_{H}H peptide monomers have binding specificity for an epitope of Clostridium difficile toxin A (TcdA) or toxin B (TcdB). This binding agent is termed “tetra-specific” as it recognizes four different toxin epitopes. It is termed “octameric” as it bears eight V_{H}H peptide monomers (two copies of the first monomer, two copies of the second monomer, two copies of the third monomer, and two copies of the fourth monomer).

In this sub-embodiment, the first, second, third and fourth V_{H}H peptide monomers each has binding specificity for a different epitope.

In certain aspects of this sub-embodiment, two of the V_{H}H peptide monomers have binding specificity for epitopes of TedA and two of the V_{H}H peptide monomers have binding specificity for epitopes of TcdB.

In certain aspects of this sub-embodiment, the V_{H}H peptide monomers independently have binding specificity for an epitope in the glucosyltransferase domain, cysteine protease domain, translocation domain or receptor binding domain of TedA or TcdB.

In a specific aspect of this sub-embodiment, the light (kappa) chain of the binding agent comprises the amino acid sequence set forth in SEQ ID NO:46 (AA6/E3 kappa) or a sequence variant having at least 95% sequence identity thereto, and the heavy chain of the binding agent comprises the amino acid sequence set forth in SEQ ID NO:44 (AH3/5D heavy) or a sequence variant having at least 95% sequence identity thereto. As this binding agent is an IgG-based binding agent, it will be clear to the skilled artisan that two heavy chain polypeptides and two light chain polypeptides, having the noted amino acid sequences, will assemble through disulfide bonding to provide the complete binding agent. The sequence variants retain TcdA and/or TedB binding specificity, or the sequence variants retain toxin-neutralizing activity, or both. The variant amino acids of the sequence variants may be located in framework regions of the V_{H}H peptide monomers.

In a second sub-embodiment, the invention is directed to bi-specific or tetra-specific, tetrameric binding agents comprising an IgG antibody and first, second, third and fourth V_{H}H peptide monomers, wherein the IgG antibody comprises two arms, each arm comprising a heavy chain lacking a variable region and a light chain lacking a variable region, and each chain having an amino terminus, wherein for a first arm of the antibody, the first V_{H}H peptide monomer is joined to the amino terminus of the light chain, and the second V_{H}H peptide monomer is joined to the amino terminus of the heavy chain, wherein for a second arm of the antibody, the third V_{H}H peptide monomer is joined to the amino terminus of the light chain, and the fourth V_{H}H peptide monomer is joined to the amino terminus of the heavy chain, and wherein the V_{H}H peptide monomers have binding specificity for an epitope of Clostridium difficile toxin A (TcdA) or toxin B (TcdB). When the binding agent is “tetra-specific”, it recognizes four different toxin epitopes; when “bi-specific” it recognizes two different toxin epitopes. The binding agents are “tetrameric” as they bear four V_{H}H peptide monomers (when bi-specific, the first and third monomer have the same sequence and bind the same epitope, and the second and fourth monomers have the same sequence and bind the same epitope; when tetra-specific, each of the monomers has a different sequence and binds a different epitope).

When the binding agent is bi-specific, the first and second monomers have binding specificity for different epitopes, the first and third monomers have identical amino acid sequences, and the second and fourth monomers have identical amino acid sequences. One of the V_{H}H peptide monomers may have binding specificity for an epitope of TcdA and one of the V_{H}H peptide monomers may have binding specificity for an epitope of TcdB.

When the binding agent is tetra-specific, each of the V_{H}H peptide monomers has binding specificity for a different epitope. Two of the V_{H}H peptide monomers may have binding specificity for epitopes of TcdA and two of the V_{H}H peptide monomers may have binding specificity for epitopes of TcdB.

In certain aspects of this sub-embodiment, each of the V_{H}H peptide monomers has binding specificity for epitopes of TcdA.

In certain aspects of this sub-embodiment, each of the V_{H}H peptide monomers has binding specificity for epitopes of TcdB.

In certain aspects of this sub-embodiment, the V_{H}H peptide monomers independently have binding specificity for an epitope in the glucosyltransferase domain, cysteine protease domain, translocation domain or receptor binding domain of TedA or TcdB.

In a specific aspect of this sub-embodiment, the light (kappa) chain of the binding agent comprises the amino acid sequence set forth in SEQ ID NO:40 (AA6 kappa) or a sequence variant having at least 95% sequence identity thereto, and the heavy chain of the binding agent comprises the amino acid sequence set forth in SEQ ID NO:36 (AH3 heavy) or a sequence variant having at least 95% sequence identity thereto. As this binding agent is an IgG-based binding agent, it will be clear to the skilled artisan that two heavy chain polypeptides and two light chain polypeptides, having the noted amino acid sequences, will assemble through disulfide bonding to provide the complete binding agent. The sequence variants retain TedA and/or TcdB binding specificity, or the sequence variants retain toxin neutralizing activity, or both. The variant amino acids of the sequence variant may be located in framework regions of the V_{H}H peptide monomers.

In another specific aspect of this sub-embodiment, the light (kappa) chain of the binding agent comprises the amino acid sequence set forth in SEQ ID NO:42 (E3 kappa) or a sequence variant having at least 95% sequence identity thereto, and the heavy chain of the binding agent comprises the amino acid sequence set forth in SEQ ID NO:38 (5D heavy) or a sequence variant having at least 95% sequence identity thereto. As this binding agent is an IgG-based binding agent, it will be clear to the skilled artisan that two heavy chain polypeptides and two light chain polypeptides, having the noted amino acid sequences, will assemble through disulfide bonding to provide the complete binding agent. The sequence variants retain TcdA and/or TcdB binding specificity, or the sequence variants retain toxin neutralizing activity, or both. The variant amino acids of the sequence variants may be located in framework regions of the V_{H}H peptide monomers.

In certain aspects of this embodiment and the sub-embodiments, the binding agents bind to TcdA and/or TcdB with specificity. In certain aspects of this embodiment, the binding agents exhibit TcdA and/or TcdB neutralizing activity.

In a third embodiment, the invention is directed to binding agents comprising V_{H}H peptide monomers joined to antibody Fc domains, where the binding agents bind TcdA and/or TcdB. In these Fc domain-based binding agents, one, two, three, four or more of the V_{H}H peptide monomers are joined to the hinge, C_{H}2 and C_{H}3 regions of each arm of Fc domain of an antibody heavy chain. Thus, the peptide monomers replace the Fab regions of an antibody.

In certain aspects of this embodiment, these binding agents comprise two, three, four, or more linked V_{H}H peptide monomers joined to the amino termini of the arms of the Fc domains. The V_{H}H peptide monomers include, but are not limited to, the V_{H}H peptide monomers 5D (SEQ ID NO:1), E3 (SEQ ID NO:3), AA6 (SEQ ID NO:5) and AH3 (SEQ ID NO:7).

In aspects of this embodiment where two or more monomer are linked, the monomers may be linked by flexible peptide linkers, generally comprising between 10 and 20 amino acids. Suitable linkers include, but are not limited to, linker-1 (SEQ ID NO:9), linker-2 (SEQ ID NO: 11), and linker-3 (SEQ ID NO:13).

In a first sub-embodiment, the invention is directed to tetra-specific, octameric binding agents comprising an antibody Fc domain and two sets of linked first, second, third and fourth V_{H}H peptide monomers, wherein the antibody Fc domain comprises two arms, each arm comprising hinge, C_{H}2 and C_{H}3 regions of an antibody heavy chain, and each arm having an amino terminus, wherein for each arm of the Fc domain, one set of linked first, second, third and fourth V_{H}H peptide monomers is joined to the amino terminus of the arm, and where the V_{H}H peptide monomers have binding specificity for an epitope of Clostridium difficile toxin A (TcdA) or toxin B (TcdB). This binding agent is termed “tetra-specific” as it recognizes four different toxin epitopes. It is termed “octameric” as it bears eight V_{H}H peptide monomers (two copies of the first monomer, two copies of the second monomer, two copies of the third monomer, and two copies of the fourth monomer).

In certain aspects of this sub-embodiment, the first, second, third and fourth V_{H}H peptide monomers each has binding specificity for a different epitope.

In certain aspects of this sub-embodiment, two of the V_{H}H peptide monomers have binding specificity for epitopes of TcdA and two of the V_{H}H peptide monomers have binding specificity for epitopes of TcdB.

In certain aspects of this sub-embodiment, the V_{H}H peptide monomers independently have binding specificity for an epitope in the glucosyltransferase domain, cysteine protease domain, translocation domain or receptor binding domain of TedA or TcdB.

In a specific aspect of this sub-embodiment, the binding agent comprises the amino acid sequence set forth in SEQ ID NO:22 (ABAB-Fc) or a sequence variant having at least 95% sequence identity thereto, where the sequence variant retains TcdA and/or TcdB binding specificity, or the sequence variant retains toxin neutralizing activity, or both. As this binding agent is an Fc domain-based binding agent, it will be clear to the skilled artisan that two identical polypeptides, having the noted amino acid sequence, serve as the arms of the binding agent and that the arms will assemble through disulfide bonding to provide the complete binding agent. The variant amino acids of the sequence variant may be located in framework regions of the V_{H}H peptide monomers.

In a second sub-embodiment, the invention is directed to bi-specific, tetrameric binding agents comprising an antibody Fc domain and two sets of linked first and second V_{H}H peptide monomers, wherein the antibody Fc domain comprises two arms, each arm comprising hinge, C_{H}2 and C_{H}3 regions of an antibody heavy chain, and each arm having an amino terminus, wherein for each arm of the Fc domain, one set of linked first and second V_{H}H peptide monomers is joined to the amino terminus of the arm, and where the V_{H}H peptide monomers have binding specificity for an epitope of Clostridium difficile toxin A (TcdA) or toxin B (TcdB). This binding agent is termed “bi-specific” as it recognizes two different toxin epitopes. It is termed “tetrameric” as it bears four V_{H}H peptide monomers (two copies of the first monomer, and two copies of the second monomer).

In certain aspects of this sub-embodiment, the first and second V_{H}H peptide monomers have binding specificity for the same or different epitopes.

In certain aspects of this sub-embodiment, the V_{H}H peptide monomers independently have binding specificity for an epitope in the glucosyltransferase domain, cysteine protease domain, translocation domain or receptor binding domain of TedA or TcdB.

In a specific aspect of this sub-embodiment, the binding agent comprises the amino acid sequence set forth in SEQ ID NO:32 (AH3/5D-Fc) or a sequence variant having at least 95% sequence identity thereto, where the sequence variant retains TedA and/or TedB binding specificity, or the sequence variant retains toxin neutralizing activity, or both. As this binding agent is an Fc domain-based binding agent, it will be clear to the skilled artisan that two identical polypeptides, having the noted amino acid sequence, serve as the arms of the binding agent and that the arms will assemble through disulfide bonding to provide the complete binding agent. The variant amino acids of the sequence variant may be located in framework regions of the V_{H}H peptide monomers.

In another specific aspect of this sub-embodiment, the binding agent comprises the amino acid sequence set forth in SEQ ID NO:34 (AA6/E3-Fc) or a sequence variant having at least 95% sequence identity thereto, where the sequence variant retains TcdA and/or TcdB binding specificity, or the sequence variant retains toxin neutralizing activity, or both. As this binding agent is an Fc domain-based binding agent, it will be clear to the skilled artisan that two identical polypeptides, having the noted amino acid sequence, serve as the arms of the binding agent and that the arms will assemble through disulfide bonding to provide the complete binding agent. The variant amino acids of the sequence variant may be located in framework regions of the V_{H}H peptide monomers.

In certain aspects of this embodiment and the sub-embodiments, the binding agents bind to TcdA and/or TcdB with specificity. In certain aspects of this embodiment, the binding agents exhibit TcdA and/or TcdB neutralizing activity.

The invention includes humanized variants of each the binding agents provided in the various embodiments and aspects defined herein. Likewise, the invention includes epitope binding fragments of each the binding agents provided in the various embodiments and aspects defined herein.

### Polynucleotides, Expression Vectors, and Host Cells

The invention includes polynucleotides comprising nucleotide sequences encoding each the binding agents provided in the various embodiments and aspects defined herein, as well as complementary strands thereof. The invention also includes expression vectors (e.g., bacterial and yeast) comprising the polynucleotides, and host cells (e.g., bacterial, yeast, mammalian, insect) comprising the expression vectors. The invention further includes methods of producing the binding agents define herein, comprising culturing the host cells under conditions promoting expression of the binding agents encoded by the expression vectors, and recovering the binding agents from the cell cultures.

Engineered Strains of S. boulardii

In a fourth embodiment, the invention is directed to strains of Saccharomyces yeast, such as S. cerevisiae and S. boulardii, engineered to produce one or more of the binding agents defined herein. In preferred aspects, the engineered strains of Saccharomyces yeast secrete the binding agents.

The identity of the Saccharomyces yeast strain is only limited in that it can be engineered to produce, and preferably secrete, one or more of the binding agents of the invention. In preferred aspects of the invention, the strain of Saccharomyces yeast engineered to produce one or more of the binding agents is S. cerevisiae or S. boulardii. The invention thus encompasses an engineered strain of S. cerevisiae that produces one or more of the binding agents defined herein, as well as an engineered strain of S. cerevisiae that secretes one or more of the binding agents defined herein. The invention also encompasses an engineered strain of S. boulardii that produces one or more of the binding agents defined herein, as well as an engineered strain of S. boulardii that secretes one or more of the binding agents defined herein.

In an example of this embodiment, the invention is directed to engineered strains of Saccharomyces yeast that produce a binding agent comprising a V_{H}H peptide monomer or linked groups of V_{H}H peptide monomers comprising two, three, four, or more monomers, each of which binds TcdA and/or TcdB, preferably with specificity. Thus, the invention encompasses engineered strains of Saccharomyces yeast that produces V_{H}H peptide binding agents comprising at least one V_{H}H peptide monomer, wherein each V_{H}H peptide monomer has binding specificity for an epitope of C. difficile toxin A (TcdA) or toxin B (TcdB). In certain aspects, these binding agents comprise two, three, four, or more linked V_{H}H peptide monomers. The V_{H}H peptide monomers include, but are not limited to, the V_{H}H peptide monomers 5D (SEQ ID NO:1), E3 (SEQ ID NO:3), AA6 (SEQ ID NO:5), and AH3 (SEQ ID NO:7).

In another example of this embodiment, the invention is directed to engineered strains of Saccharomyces yeast that produce binding agents comprising V_{H}H peptide monomers joined to IgG antibodies, where the binding agents bind TcdA and/or TcdB, as defined herein. In these IgG-based binding agents, the variable regions of the light and heavy chains of IgG antibodies are replaced by one, two, three, four or more of the V_{H}H peptide monomers.

In further example of this embodiment, the invention is directed to engineered strains of Saccharomyces yeast that produce binding agents comprising V_{H}H peptide monomers joined to antibody Fc domains, where the binding agents bind TcdA and/or TcdB, as defined herein. In these Fc domain-based binding agents, one, two, three, four or more of the V_{H}H peptide monomers are joined to the hinge, C_{H}2 and C_{H}3 regions of each arm of Fc domain of an antibody heavy chain. Thus, the peptide monomers replace the Fab regions of an antibody.

In yet another example of this embodiment, the invention is directed to an engineered strain of Saccharomyces yeast that produces a tetra-specific, tetrameric binding agent, wherein the binding agent comprises linked first, second, third and fourth V_{H}H peptide monomers, and wherein the V_{H}H peptide monomers independently have binding specificity for an epitope of Clostridium difficile toxin A (TcdA) or toxin B (TcdB). In certain aspects, the first, second, third and fourth V_{H}H peptide monomers each has binding specificity for a different epitope. In certain aspects, the two of the V_{H}H peptide monomers have binding specificity for epitopes of TcdA and two of the V_{H}H peptide monomers have binding specificity for epitopes of TcdB. In certain aspects, the V_{H}H peptide monomers independently have binding specificity for an epitope in the glucosyltransferase domain, cysteine protease domain, translocation domain or receptor binding domain of TedA or TcdB.

In a preferred example of this embodiment, the invention is directed to an engineered strain of yeast, wherein the binding agent is ABAB, wherein the first and third monomers have binding specificity for epitopes of TcdA and the first and third monomers are V_{H}H peptide monomers AH3 (SEQ ID NO:7) and AA6 (SEQ ID NO:5), respectively, and wherein the second and forth monomers have binding specificity for epitopes of TcdB and the second and forth monomers are V_{H}H peptide monomers 5D (SEQ ID NO:1) and E3 (SEQ ID NO:3), respectively. In certain aspects, the ABAB binding agent comprises the amino acid sequence set forth in SEQ ID NO: 19, or a sequence variant having at least 95% sequence identity thereto, wherein the sequence variant retains TcdA and/or TcdB binding specificity, or the sequence variant retains toxin neutralizing activity, or both. In certain aspects, the ABAB binding agent further comprises an N-terminal secretion signal selected from the AT secretion signal (MRFPSIFTAVLFAASSALA (SEQ ID NO:99)) and the IVS secretion signal (MLLQAFLFLLAGFAAKISA (SEQ ID NO:103)).

In certain aspects, the ABAB binding agent is expressed from a plasmid within the yeast, wherein the ABAB binding agent comprises the amino acid sequence set forth in SEQ ID NO: 107, or a sequence variant having at least 95% sequence identity thereto, and wherein the sequence variant retains TcdA and/or TcdB binding specificity, or the sequence variant retains toxin neutralizing activity, or both. The plasmid may be, but is not limited to, pCEV-URA3-TEF-AT-yABAB-cMyc (SEQ ID NO:88).

In certain aspects, the ABAB binding agent coding sequence is integrated into a chromosome of the strain of yeast, wherein the ABAB binding agent comprises the amino acid sequence set forth in SEQ ID NO:109, or a sequence variant having at least 95% sequence identity thereto, and wherein the sequence variant retains TcdA and/or TedB binding specificity, or the sequence variant retains toxin neutralizing activity, or both.

Aspects of this embodiment include engineered strains of Saccharomyces yeast that produce a therapeutic protein having binding specificity for a unique epitope of Clostridium difficile toxin A (TcdA) or toxin B (TcdB), or both. Preferably, the engineered strain of Saccharomyces yeast is S. cerevisiae or S. boulardii. A therapeutic protein is any protein that can bring about an improvement or cure in a medical condition in a subject, or that can inhibit or prevent a medical condition from developing in a subject. Suitable therapeutic protein include, but are not limited to, proteins that (a) replace a protein that is deficient or abnormal; (b) augment an existing pathway; (c) provide a novel function or activity; (d) interfere with a molecule or organism; and (e) deliver other compounds or proteins, such as a radionuclide, cytotoxic drug, or effector proteins. Therapeutic proteins also include antibodies and antibody-based drugs, Fc fusion proteins, anticoagulants, blood factors, bone morphogenetic proteins, engineered protein scaffolds, enzymes, growth factors, hormones, interferons, interleukins, and thrombolytics. Therapeutic proteins further include bispecific monoclonal antibodies (mAbs) and multispecific fusion proteins, mAbs conjugated with small molecule drugs, and proteins with optimized pharmacokinetics.

Methods of Making Engineered Strains of S. boulardii

The invention is also directed to methods of making strains of Saccharomyces yeast engineered to produce one or more of the binding agents defined herein.

The invention thus encompasses a method of preparing a strain of Saccharomyces yeast engineered to produce one or more of the binding agents defined herein comprising (a) transforming a strain of Saccharomyces yeast with an expression vector encoding the binding agent, and (b) screening the yeast of (a) for production of the binding agent. In a certain aspect, the expression vector is plasmid pCEV-URA3-TEF-AT-yABAB-cMyc (SEQ ID NO:88).

The invention thus encompasses a method of preparing a strain of Saccharomyces yeast engineered to produce one or more of the binding agents defined herein comprising (a) chromosomally integrating a polynucleotide sequence encoding the binding agent into the genome of the strain of Saccharomyces yeast, and (b) screening the yeast of (a) for production of the binding agent. In certain aspects, the chromosomal integration is performed via:
(a) amplifying a polynucleotide sequence encoding the ABAB binding agent from plasmid pCEV-G4-Km-TEF-AT-yABAB hAA6T83N-tagless (SEQ ID NO:90) using primers containing (i) nucleic acid sequence homologous to a selected yeast chromosomal integration site and (ii) nucleic acid sequence homologous to regions 5′ and 3′ of ABAB binding agent coding sequence of the plasmid, to produce an integration cassette,
(b) transforming yeast with the integration cassette produced in (a) with pCRI-Sb-81 (SEQ ID NO:91) or pCRI-Sb-82 (SEQ ID NO:92) to induce a double stranded break within the corresponding yeast chromosomal delta sites under conditions promoting spontaneous integration of the integration cassette into the site of the double stranded break,
(c) screening the transformed yeast of (b) for production of the ABAB binding agent.

In certain aspects of these methods, the strain of Saccharomyces yeast engineered to produce the binding agents is an auxotrophic strain of Saccharomyces yeast, such as a ura3-strain of yeast. A ura3-strain of yeast can be utilized under ura3 selection.

In certain aspects of these methods, the strain of Saccharomyces yeast engineered to produce the binding agents is S. cerevisiae or S. boulardii.

In certain aspects of these methods, the screening is performed using an immunoassay, such as an ELISA.

### Pharmaceutical Formulations

The invention includes pharmaceutical formulations comprising one or more of the binding agents defined herein and a pharmaceutically acceptable carrier or diluent. The invention also includes pharmaceutical formulations comprising one or more of the engineered strains of Saccharomyces yeast defined herein and a pharmaceutically acceptable carrier or diluent. In certain aspects, the Saccharomyces yeast is S. cerevisiae or S. boulardii.

### Methods of Treating and Preventing

In a sixth embodiment, the invention is directed to methods of treating or preventing a disease symptom induced by C. difficile in a subject comprising administering a therapeutically-effective amount of one or more binding agents and/or one or more engineered strains of Saccharomyces yeast as defined herein to a subject having C. difficile infection or a risk of developing C. difficile infection. In preferred aspects, the Saccharomyces yeast is S. cerevisiae or S. boulardii.

In certain aspects of this embodiment, the disease symptom induced by C. difficile is diarrhea.

In a seventh embodiment, the invention is directed to methods of neutralizing C. difficile toxin TcdA and/or TedB in a subject infected by C. difficile comprising administering a therapeutically-effective amount of one or more binding agents and/or one or more engineered strains of Saccharomyces yeast as defined herein to a subject having C. difficile infection. In preferred aspects, the Saccharomyces yeast is S. cerevisiae or S. boulardii.

In an eighth embodiment, the invention is directed to methods of treating or preventing C. difficile infection in a subject comprising administering a therapeutically-effective amount of one or more of the binding agents and/or one or more engineered strains of Saccharomyces yeast as defined herein to a subject having C. difficile infection or a risk of developing C. difficile infection. In preferred aspects, the Saccharomyces yeast is S. cerevisiae or S. boulardii. In certain aspects of the eighth embodiment, the method further comprises administering a therapeutically-effective amount of an antibiotic to the subject.

In a ninth embodiment, the invention is directed to methods of maintaining normal bowel function in a subject having a C. difficile infection comprising administering a therapeutically-effective amount of one or more of the binding agents and/or one or more engineered strains of Saccharomyces yeast as defined herein to a subject having C. difficile infection or a risk of developing C. difficile infection. In preferred aspects, the Saccharomyces yeast is S. cerevisiae or S. boulardii. In certain aspects of the ninth embodiment, the method further comprises administering a therapeutically-effective amount of an antibiotic to the subject.

In certain aspects of the methods, the binding agent is in a pharmaceutical formulation comprising the binding agent and a pharmaceutically acceptable carrier or diluent.

In certain aspects of the methods, the therapeutically-effective amount of the binding agent is between 10 μg/kg and 100 mg/kg of the agent per body weight of the subject.

In certain aspects of the methods, the agent is administered to the subject orally, parenterally or rectally.

In certain aspects of the methods, the engineered strain of Saccharomyces yeast is in a pharmaceutical formulation comprising the engineered strain and a pharmaceutically acceptable carrier or diluent. In preferred aspects, the Saccharomyces yeast is S. cerevisiae or S. boulardii.

In certain aspects of the methods, the therapeutically-effective amount of the engineered strain of Saccharomyces yeast is between 10 μg/kg and 100 mg/kg of the engineered strain per body weight of the subject. In preferred aspects, the Saccharomyces yeast is S. cerevisiae or S. boulardii.

In certain aspects of the methods, the engineered strain of Saccharomyces yeast is administered to the subject orally, nasally or rectally. In preferred aspects, the Saccharomyces yeast is S. cerevisiae or S. boulardii.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits of the invention.

### BRIEF DESCRIPTION OF DRAWINGS

FIG. 1. Illustration of strategies for making binding agents of the invention.

FIG. 2. A diagram of C. difficile toxins TcdA and TcdB, showing the glucosyltransferase domains (GT), cysteine protease domains (CPD), translocation domains (TD) and receptor binding domains (RBD) of each toxin. VHHs that recognize and bind the different toxin domains are shown. Those that are underlined are those that have toxin-neutralizing activity.

FIGS. 3A-3F. Monomeric or dimeric VHHs possess potent neutralizing activity. V_{H}Hs block cell rounding induced by TcdA (FIG. 3A) or TcdB (FIG. 3B) at nM concentrations. (FIG. 3C) Diagram of two heterodimers against TcdA or TcdB. His₍₆₎ tag on N-terminus facilitates purification; a flexible spacer (FS) separate the two V_{H}Hs. (FIG. 3D) Dimer 5D/E3 increases its neutralizing activity at least 10-fold over a simple mix of the two V_{H}Hs. Heterodimers fully protected mice from lethal ip challenge with TcdB (FIG. 3E) or TcdA (FIG. 3F).

FIG. 4. Diagram of ABAB. His-tag and E-tag are epitope tags for purification and detection, respectively. FS: flexible linker; ABP: albumin binding peptide.

FIGS. 5A-5B. ABAB is highly potent in protecting mice from C. difficile spore (FIG. 5A) and toxin (FIG. 5B) challenge. MK HuMabs: a mixture of Merck anti-TcdA (actoxumab) and anti-TcdB (bezlotoxumab) human monoclonal antibodies that are undergoing clinical trials.

FIGS. 6A-6B. Anti-toxin sera against both toxins protect mice from CDI. Mice were i.p. injected with 50 ul alpaca anti-sera against TcdA (“Anti-A”), TcdB (“Anti-B”), TcdA+TcdB (“Anti-A+Anti-B”) or with 100 ul presera or PBS (“CTR”) for 4 hours before C. difficile spore (UK1 strain, 106 spores/mouse) inoculation. Mouse survival (FIG. 6A; Anti-A+Anti-B vs. PBS, p=0.006) and weight loss (FIG. 6B) are illustrated (*, p<0.05 between Anti-A+Anti-B vs. control).

FIG. 7. The diagram of the ABAB and ABAB-IgG molecules.

FIGS. 8A-8B. ELISA analysis of binding of ABAB-IgG to TcdA (FIG. 8A) and TcdB (FIG. 8B) as compared with the binding of the individual V_{H}Hs to the respective toxins.

FIGS. 9A-9B. Sandwich ELISA analysis of simultaneous binding of the tetraspecific antibody IgG-ABAB to both TcdA and TcdB. FIG. 9A shows serially diluted ABAB-IgG added to ELISA plates coated with TcdA (TxA), followed by TcdB (TxB). FIG. 9B shows serially diluted ABAB-IgG added to ELISA plates coated with TcdB (TxB), followed by TcdA (TxA).

FIGS. 10A-10B. ABAB-IgG neutralizing activities against TcdA (FIG. 10A) and TcdB (FIG. 10B).

FIG. 11. Graph showing in vivo neutralizing activity of ABAB-IgG against C. difficle infection in mice versus Merck antibodies against TcdA and TcdB (actoxumab and bezlotoxumab).

FIG. 12. Design of studies on the effects of prophylactic ABAB-IgG against C. difficile infection.

FIGS. 13A-13B. Bi-specific sandwich ELISA. (FIG. 13A) A diagram of toxins and antibodies setup in ELISA. (FIG. 13B) O.D. reading of various TedA concentrations; 125 ng/ml of TcdA was chosen for subsequence ELISA.

FIGS. 14A-14B. Activity of ABAB secreted by Sc-ABAB. (FIG. 14A) Neutralizing effect of secreted ABAB in S. cerevisiae culture supernatant. Sc: S. cerevisiae (BY4741); Sc-ABAB: S. cerevisiae (BY4741)-pD1214-FAKS-ABAB; r-ABAB: recombinant ABAB. ABAB in the supernatant of Sc-ABAB is able to fully protect cells from intoxication. ELISA O.D. readings of supernatants from individual Sc-ABAB clones (FIG. 14B).

FIGS. 15A-15B. ABAB secretion level with various secretion signals. (FIG. 15A) ABAB secretion measured by ELISA and normalized against cell density based on O.D. 600 in S. cerevisiae. Statistical significance was determined by Kruskal-Walls test followed by Dunn's Multiple comparison test. * p<0.05 ** p<0.01 (FIG. 15B) ABAB secretion measured by ELISA and normalized against cell density based on O.D. 600 in S. boulardii. Statistical significance was determined by Mann Whitney test. **** p<0.0001.

FIG. 16. A diagram of targeted deletion of chromosomally encoded genes by homologous recombination in S. boulardii.

FIGS. 17A-17D. S. boulardii URA34/4 expressing ABAB. (FIG. 17A) Growth comparison in YPD containing vancomycin (1 mg/ml) versus without. (FIG. 17B) ABAB stability in S. boulardii culture supernatant after 2 hours of incubation determined by ELISA. (FIG. 17C) Neutralizing activity of ABAB from the culture supernatant of S. boulardii URA34/4 expressing ABAB. (FIG. 17D) ABAB detection in S. boulardii URA34/4 expressing ABAB culture supernatant by western blot. Enriched: ABAB contains c-Myc tag at the end of C-terminus and was further concentrated using α-c-Myc tag antibodies.

FIGS. 18A-18C. Protection of S. boulardii expressing ABAB in CDI prevention in mice. (FIG. 18A) Survival rate, (FIG. 18B) Weight loss, (FIG. 18C) Diarrhea incident, throughout the course of infection were recorded and presented. * significance as determined by Fisher's exact test with two tailed and 95% confidence interval; p value is 0.0108 for FIG. 18A and regular two-way ANOVA (not repeated measures) followed by Dunnett's multiple comparison test was used for FIG. 18B and FIG. 18C, * P≤0.05. “Sb: EP” is S. boulardii with the empty plasmid; “Sb: ABAB” is S. boulardii expressing ABAB.

FIGS. 19A-19C. Protection of S. boulardii expressing ABAB in treating CDI mice. (FIG. 19A) Survival rate, (FIG. 19B) Weight loss, (FIG. 19C) Diarrhea incident, throughout the course of infection were recorded and presented. * significance as determined by Fisher's exact test with two tailed and 95% confidence interval; p value is 0.0256 for FIG. 19A; regular two-way ANOVA (not repeated measures) followed by Dunnett's multiple comparison test for FIG. 19B and FIG. 19C. * P≤0.05 ** P≤0.01 **** P≤0.0001 for FIG. 19B and FIG. 19C. “Sb: EP” is S. boulardii with the empty plasmid; “Sb: ABAB” is S. boulardii expressing ABAB.

FIGS. 20A-20C. Protection of S. boulardii expressing ABAB in CDI recurrent mice. (FIG. 20A) Survival rate, (FIG. 20B) Weight loss, (FIG. 20C) Diarrhea incident, throughout the course of infection were recorded and presented. * significance as determined by Fisher's exact test with two tailed and 95% confidence interval; p value is 0.017 for FIG. 20A; regular two-way ANOVA (not repeated measures) followed by Dunnett's multiple comparison test for FIG. 20B and FIG. 20C. * P≤0.05 *** P≤0.001 **** P≤0.0001 for FIG. 20B and FIG. 20C. “Sb: EP” is S. boulardii with the empty plasmid; “Sb: ABAB” is S. boulardii expressing ABAB.

FIG. 21. A diagram of 8 site-targeted chromosomal integration using CRISPR. Ty1-H3 (Genbank accession no. M18706) was used to blast against draft genome of MYA796 to obtain 8 site sequences. Compiled sequences were used to identify common protospacer adjacent motif (PAM) sites and protospacers. Two PAM site sequences were chosen based on best coverage for multiple sites and common homologous sequences located upstream and downstream of the protospacer and PAM sites for simple integration of ABAB expression cassette. PAM site “I” is provided in SEQ ID NO:93; PAM site “II” is provided in SEQ ID NO: 94. Homologous recombination sequences used in primers to generate ABAB expression cassette by PCR are underlined.

FIGS. 22A-22B. ABAB secretion of S. boulardii using CRISPR-based targeting δ site chromosomal integration. (FIG. 22A) ABAB secretion measured by ELISA. ITG: ABAB integration cassette. Low: CRISPR plasmid to ITG ratio at 2; High: CRISPR plasmid to ITG ratio at 0.25. (FIG. 22B) ABAB secretion amount comparison. M−/−^{Cir0}: pKC, M−/−^{Cir+}: ABAB, M−/−^{Cir0}: ABAB are plasmid based. Ch^{Ins}: single site target chromosomal integration of ABAB cassette through conventional homologous recombination. C^{RISPR}1-2: ABAB cassette integration at site I. C^{RISPR}3-4: ABAB cassette integration at site II.

FIGS. 23A-23C. Protection of S. boulardii expressing ABAB in treating CDI mice. (FIG. 23A) Survival rate, (FIG. 23B) Weight loss, (FIG. 23C) Diarrhea incident, throughout the course of infection were recorded and presented. * significance as determined by Fisher's exact test with two tailed and 95% confidence interval; p value is 0.0325 for (FIG. 23A); regular two-way ANOVA (not repeated measures) followed by Dunnett's multiple comparison test for FIG. 23B and FIG. 23C. * P≤0.05 ** P≤0.01 for FIG. 23B and FIG. 23C.

### DETAILED DESCRIPTION OF THE INVENTION

### I. Definitions

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

C. difficile-associated disease (CDI) is mainly caused by two large exotoxins, namely toxin A (TcdA) and toxin B (TcdB), produced by the bacteria. These toxins are structurally similar, large, single-chain proteins (TcdA is about 300 kD; TcdB is about 270 kD) that exhibit similar modes of action on host cells. Both toxins target host Rho GTPases, leading to enzyme inactivation, followed by cytoskeleton disorganization and apoptosis. In intestinal epithelial cells, TcdA catalyzes glucosylation of the Rho GTPases, leading to reorganization of the actin cytoskeleton with accompanying morphological changes such as complete rounding of cells and destruction of the intestinal barrier function. The toxins can individually cause CDI in animals, and TcdA TcdB-strains of the bacteria are avirulent.

Numerous independent studies have demonstrated that neutralizing antibodies against the toxins confer protection against CDI [24-33]. Because TcdA and TcdB are essential virulence factors for C. difficile, neutralizing antibodies produced against both toxins protect against toxigenic C. difficile infection in animal models [30-33]. In humans, high serum levels of antitoxin antibodies are associated with reduced disease severity and incidence of relapse [9,25,29].

Therefore, a preventative rationale for systemically and orally administered antitoxin antibodies exists. However, monoclonal antibodies targeting a single epitope are typically low affinity, and use of such antibodies runs the risk of inducing mutations within the epitopes of the toxins thereby creating additional strains. Thus, neutralizing antitoxins targeting multiple, key, and conserved toxin epitopes are highly desirable.

The present invention builds on existing knowledge regarding anti-TcdA and anti-TcdB antibodies for the treatment and prevention of CDI, and the symptoms of CDI. Provided herein are antibody-based, fusion protein binding agents derived from human and camelid immunoglobulins, optionally expressed by the probiotic yeast Saccharomyces strain in a subject. These binding agents recognize and bind with specificity to C. difficile TcdA and/or TedB. Some of these binding agents exhibit toxin-neutralizing activity. These yeast-based immunotherapeutic can be used to treat or prevent primary and recurrent CDI, as well as the symptoms of primary and recurrent CDI. In preferred aspects, the Saccharomyces yeast is S. cerevisiae or S. boulardii.

As discussed in detail below, camelid animals (dromedary camels, Bactrian camels, wild Bactrian camels, llamas, alpacas, vicuñas, and guanacos) produce a class of functional immunoglobulins that lack light chains and are thus heavy chain-only antibodies (HCAbs) with binding properties equivalent to those achieved by conventional IgG [35]. The V_{H} domain of HCAbs, called V_{H}H, is similar to the conventional human V_{H} domain but has unique sequence and structural characteristics [36]. DNA encoding this domain can be readily cloned and expressed in microbes to yield soluble protein monomers that retain the antigen-binding properties of the parent HCAb. These V_{H}H peptide monomer binding agents are small (˜15 kDa), easy to produce, and generally more stable than conventional antibody fragments [37-39]. V_{H}Hs have being explored to treat intestinal diseases since they are relatively resistant to proteases and can be further engineered to enhance such properties [40]. They can also be produced as fusion proteins with human antibodies, such as IgG, and fragments of human antibodies, such as Fc domains.

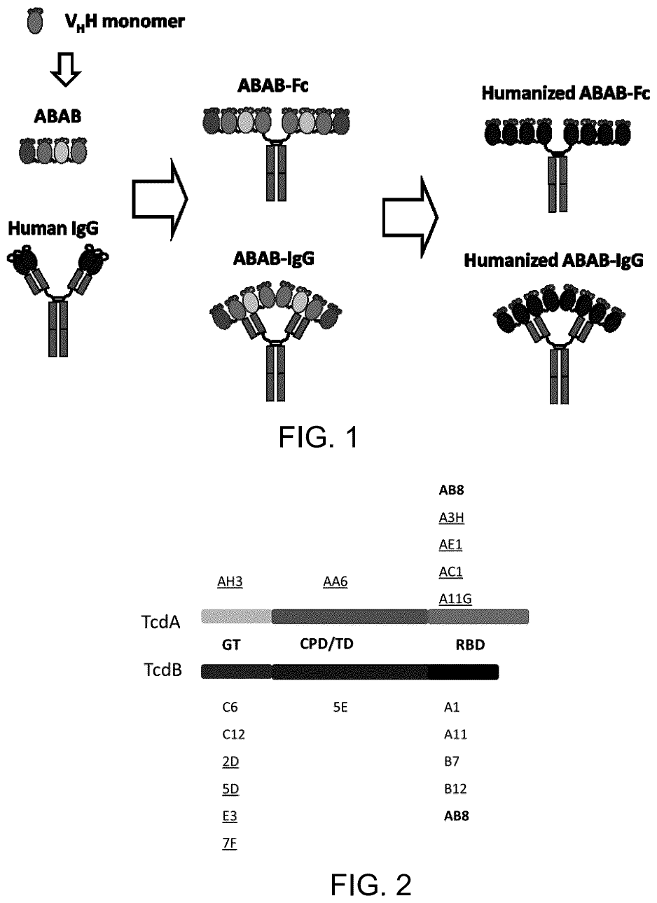

The present invention utilizes the advantageous characteristics of HCAbs in the production of binding agents that can be used in the treatment and prevention of CDI. As disclosed herein, V_{H}H peptide monomers were screened for TcdA and TcdB epitope recognition and binding. Those monomers that exhibited epitope binding and had toxin-neutralizing activity were linked to produce the binding agents of the invention. The binding agents include simple V_{H}H peptide monomers and linked groups of V_{H}H peptide monomers (comprising 2, 3, 4, or more monomers), as well as more complex binding agents that comprise V_{H}H peptide monomers joined to antibody Fc domains, as well as V_{H}H peptide monomers joined to IgG antibodies (see FIG. 1).

Further, Saccharomyces boulardii, a Generally Regarded as Safe (GRAS) organism by the FDA, is commonly available over-the-counter for use in promoting intestinal health and amelioration of gastrointestinal illness due to diarrheal diseases. This yeast strain has been studied in multiple randomized double-blinded placebo-controlled clinical trials for both safety and efficacy against intestinal diseases including CDI [42-46]. S. boulardii treatment significantly reduced CDI recurrence [44-46], and those recurrent patients had significantly less S. boulardii in stools than non-recurring patients [43]. The immune modulatory effects of S. boulardii that provide protection against C. difficile toxin-induced inflammation have been described [47-49]. In addition, S. boulardii may help in maintaining normal microbiota [50]; a recent clinical trial (NCT01473368) found that S. boulardii treatment can prevent some antibiotic-induced microbiome changes and, in parallel, can reduce antibiotic-associated diarrhea.

S. cerevisiae (commonly known as “brewer's yeast”), which is genetically related to S. boulardii, has been used successfully to express V_{H}Hs with high yield [51]. S. boulardii is physiologically distinct from S. cerevisiae, although genome analysis has revealed that both genomes are remarkably similar at the nucleotide level [52,53]. Therefore, molecular genetic tools previously developed for use in S. cerevisiae are now being used with S. boulardii [54-56], making this probiotic a candidate for engineering as a therapeutic agent against CDI.

There are several additional metabolic characteristics which make S. boulardii ideal for use as an oral therapeutic agent. In contrast to S. cerevisiae, S. boulardii grows well at 37° C. and it is more resistant to acidic environmental conditions [57], making this strain particularly well suited for better surviving and persisting in the human intestinal tract after oral administration. In addition, an experimental murine oral colonization model with Saccharomyces is well characterized [58]; using this model, protection has been reported against oral challenge with enteric pathogens such as Salmonella Typhimurium [58,59] and Enteritidis in conventional mice orally treated with S. boulardii, as well as protection against CDI challenge in pretreated gnotobiotic animals [58,61]. The probiotic S. boulardii, genetically engineered to secrete V_{H}H binding agents capable of neutralizing both TcdA and TcdB of C. difficile, could significantly improve the therapeutic capacity of this probiotic to disrupt both ongoing and recurrent CDI.

In view of the exceptional characteristics of S. boulardii, strains of S. boulardii expressing the binding agents defined herein where produced and tested. As described in the Examples, these yeast-based immunotherapeutics can be used to treat or prevent primary and recurrent CDI, as well as the symptoms of primary and recurrent CDI.

V_{H}H Monomers & V_{H}H Heterodimers

As initially reported in WO 16/127104, the inventors established an efficient platform to screen V_{H}H monomers against specific domains of both C. difficile toxins. Using highly immunogenic atoxic holotoxins for immunization, and bioactive chimeric toxins (with normal domain functions) for screening, panels of V_{H}H monomers binding to different domains of TedA or TcdB were prepared. A majority of these V_{H}H monomers possessed potent neutralizing activity and their binding to specific domains of TedA and TcdB was determined (FIG. 2).

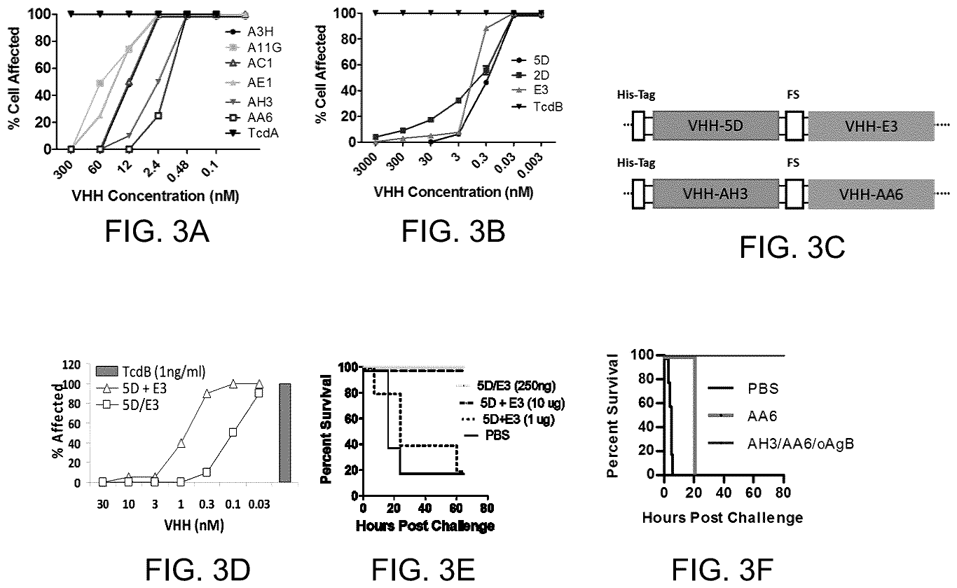

Several of the V_{H}H monomers bind to highly conserved TcdA/TcdB epitopes. For example, the E3 V_{H}H monomer binds to the Rho GTPase binding site and blocks glucosylation; the AH3 V_{H}H monomer binds to the GT domain of the toxin; the 7F V_{H}H monomer binds to cysteine protease cleavage sites and blocks GT domain cleavage and release. Some V_{H}H monomers have potent toxin neutralizing activity, capable of blocking toxin cytotoxic activity at nM concentrations (monomers underlined in FIG. 2; see also FIGS. 3A and 3B). Table 1 references amino and nucleic acid sequences in the Sequence Listing for some of these V_{H}H peptide monomers, both wild-type and codon-optimized versions. While both the optimized and non-optimized versions can be used in the production of the various binding agents of the present invention, the codon-optimized versions are preferred for expression in mammalian cells.

The present invention includes each of the V_{H}H peptide monomers referenced in Table 1 as well as sequence variants thereof having at least 85%, 86%, 87%, 88%, 89%, 90%, 91%, 92%, 93%, 94%, 95%, 96%, 97%, 98%, or 99% sequence identity over the entire length of the peptide sequence and retaining the toxin binding and/or neutralizing activity of the wild-type peptide. The present invention also includes polynucleotide sequences encoding each of the V_{H}H peptide monomers of Table 1 and the sequence variants thereof, as well as complementary strands thereof.

TABLE 1

| | | | SEQ ID | SEQ ID |
|---|---|---|---|---|
| | | | NO for | NO for |
| | | | Amino | Nucleic |
| | Codon | | Acid | Acid |
| Name | Optimized? | Location of epitope | Seq. | Seq. |
| 5D | Yes | TcdB glucosyltransferase domain | 1 | 2 |
| E3 | Yes | TcdB glucosyltransferase domain | 3 | 4 |
| AA6 | Yes | TcdA cysteine protease domain | 5 | 6 |
| AH3 | Yes | TcdA glucosyltransferase domain | 7 | 8 |
| 5D | No | TcdB glucosyltransferase domain | 48 | 49 |
| E3 | No | TcdB glucosyltransferase domain | 50 | 51 |
| AA6 | No | TcdA cysteine protease domain | 52 | 53 |
| AH3 | No | TcdA glucosyltransferase domain | 54 | 55 |

To enhance the binding activity of the peptide monomers, V_{H}H peptide homo- and hetero-dimer binding agents were created, where two V_{H}H peptide monomers are linked (FIG. 3C). Homodimer binding agents comprise two identical monomers that bind identical epitopes on two different toxins. Heterodimer binding agents comprise two different monomers that bind two distinct epitopes of the same toxin or distinct epitopes on two different toxins. The V_{H}H heterodimers were found to possess substantially enhanced neutralizing activities compared with equimolar mixtures of the individual V_{H}H peptide monomers comprising the heterodimers (FIG. 3D). Indeed, heterodimers 5D/E3 and AH3/AA6 were found to fully protect mice from lethal systemic TcdB or TcdA challenge respectively, whereas mixed 5D and E3, or AA6 alone were only partially protective (FIGS. 3E and 3F).

The V_{H}H monomers in the homo- and hetero-dimers are linked using a short, flexible linker of between 10 and 20 amino acids. Suitable linkers include those provided in Table 2. Table 2 also includes codon-optimized versions of the three linkers. While both the optimized and non-optimized versions can be used in the production of the various binding agents of the present invention, the codon-optimized versions are preferred for expression in mammalian cells.

TABLE 2

| | | SEQ ID | SEQ ID |
|---|---|---|---|
| | | NO for | NO for |
| | Codon | Amino | Nucleic |
| Name | Optimized? | Acid Seq. | Acid Seq. |
| Linker-1 | Yes | 9 | 10 |
| Linker-2 | Yes | 11 | 12 |
| Linker-3 | Yes | 13 | 14 |
| Linker-1 | No | 56 | 57 |
| Linker-2 | No | 58 | 59 |
| Linker-3 | No | 60 | 61 |

It will be understood by the skilled artisan that minor changes can be made to the sequence of the flexible linker without departing from the properties of the peptide. Sequence variants of the flexible linker having at least 85%, 86%, 87%, 88%, 89%, 90%, 91%, 92%, 93%, 94%, 95%, 96%, 97%, 98%, or 99% sequence identity over the entire length of the peptide sequence and retaining properties of the linker upon which they are based may thus be used.

The present invention includes V_{H}H peptide homodimer binding agents comprising pairs of any of the monomers listed in Table 1, linked by a flexible linker as defined above. The present invention also includes V_{H}H peptide heterodimer binding agents comprising any combination of two of the monomers listed in Table 1, linked by a flexible linker as defined above. Exemplary heterodimers are provided in Table 3.

TABLE 3

| | SEQ ID | SEQ ID |
|---|---|---|
| | NO for | NO for |
| | Amino | Nucleic |
| Name | Acid Seq. | Acid Seq. |
| AH3-5D | 15 | 16 |
| AA6-E3 | 17 | 18 |
| 5D-E3 | 62 | 63 |
| AH3-AA6 | 64 | 65 |

The present invention also includes sequence variants of the V_{H}H peptide homo- and hetero-dimers having at least 85%, 86%, 87%, 88%, 89%, 90%, 91%, 92%, 93%, 94%, 95%, 96%, 97%, 98%, or 99% sequence identity over the entire length of the protein sequence and retaining the toxin binding and/or neutralizing activity of the wild-type protein. The present invention further includes polynucleotide sequences encoding each the V_{H}H peptide homo-hetero-dimers and the sequence variants thereof, as well as complementary strands thereof.

The invention also includes V_{H}H peptide homo- and hetero-trimer binding agents where three monomers are linked using the flexible linkers defined above in Table 2. Any combination of the monomers of Table 1 may be used, including trimers comprising three copies of the same monomer, trimers comprising two copies of one monomer and a single copy of another, and trimers comprising three different monomers. Sequence variants of the V_{H}H peptide homo- and hetero-trimers are included in the invention, having at least 85%, 86%, 87%, 88%, 89%, 90%, 91%, 92%, 93%, 94%, 95%, 96%, 97%, 98%, or 99% sequence identity over the entire length of the protein sequence and retaining the toxin binding and/or neutralizing activity of the wild-type protein. The present invention further includes polynucleotide sequences encoding each the V_{H}H peptide homo- and hetero-trimers and the sequence variants thereof, as well as complementary strands thereof.

### ABAB

The success of the peptide monomers and heterodimers allowed the inventors to develop binding agents comprising four linked V_{H}H peptide monomers. This was a goal of the research as earlier work had shown that the most useful agents in the treatment and prevention of CDI would be single antibodies that can simultaneously neutralize both TcdA and TcdB as this would be necessary in order to convey full protection against most pathogenic C. difficile strains. By creating tetra-specific binding agents that recognize and bind two epitopes on each of the toxins, the binding and neutralizing activity of the proteins might be strengthened. Therefore, four domain (tetra-specific) V_{H}H binding agents were generated.

The tetra-specific, tetrameric binding agents can be prepared from any combination of the monomers of Table 1, where the monomers are linked using the flexible linkers of Table 2. These binding agents include those having four copies of the same monomer, those having three copies of the same monomer, those having two copies of the same monomer, those having four unique monomers, and variations therein. Sequence variants of the tetramers are included in the invention, having at least 85%, 86%, 87%, 88%, 89%, 90%, 91%, 92%, 93%, 94%, 95%, 96%, 97%, 98%, or 99% sequence identity over the entire length of the protein sequence and retaining the toxin binding and/or neutralizing activity of the wild-type protein. The present invention further includes polynucleotide sequences encoding each tetramer and the sequence variants thereof, as well as complementary strands thereof.

ABBA is a particular binding agent of the invention that comprises four linked V_{H}H monomers, AH3-E3-E3-AA6. ABBA thus has two identical monomers (E3) and two additional different monomers (AH3 and AA6) (See Table 1).

ABAB is another particular binding agent of the invention that comprises four linked V_{H}H monomers, each of which has binding specificity for a different epitope of TedA or TcdB. ABAB is thus a tetra-specific, tetrameric binding agent that consists of four distinct neutralizing V_{H}H monomers, two against TcdA and two against TcdB. This structural feature allows ABAB to bind simultaneously to two distinct neutralizing epitopes on each toxin. As described below, affinity/avidity and neutralizing activity of ABAB is more than 3-logs higher than human monoclonal antibodies (HuMabs) currently undergoing clinical trials for treatment of CDI.

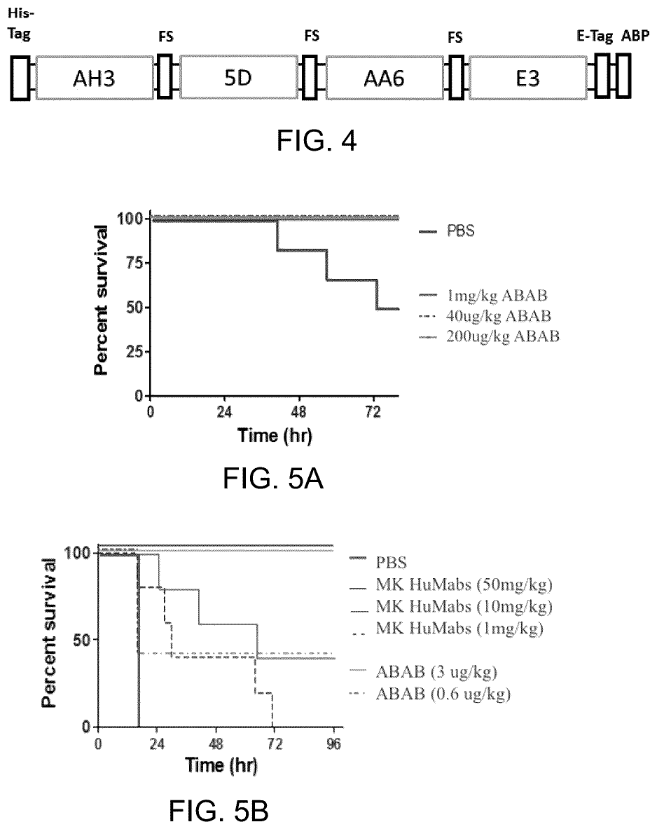

ABAB binding agent was prepared by linking V_{H}H monomers AH3, 5D, AA6, and E3 (Table 1) using flexible linkers (Table 2). This binding agent targets conserved, non-overlapping epitopes and has excellent toxin neutralizing activity. In the design of ABAB (FIG. 4), V_{H}H peptide monomers AH3 and AA6 were separated by placing the 5D between them because AH3 and AA6 bind to GT and TD respectively (FIG. 2), which are spatially distant to each other. This design allowed AH3 and AA6 to bind to TedA simultaneously.

The complete amino acid sequence comprising ABAB is provided in SEQ ID NO:19; the nucleic acid sequence encoding the protein is provided in SEQ ID NO:20. The present invention thus includes the ABAB binding agent provided in SEQ ID NO:19, as well as sequence variants of the ABAB binding agent having at least 85%, 86%, 87%, 88%, 89%, 90%, 91%, 92%, 93%, 94%, 95%, 96%, 97%, 98%, or 99% sequence identity over the entire length of the protein sequence and retaining the toxin binding and/or neutralizing activity of the wild-type protein. The sequence variants include variants wherein the variant is humanized and/or wherein the amino acids are optimized for production and secretion by yeast.

The present invention further includes polynucleotide sequences encoding the ABAB binding agent (e.g., SEQ ID NO:20) and the sequence variants thereof, as well as complementary strands thereof.

Modified versions of the ABAB binding agent encompassed by the invention includes those having one or more of (i) a His₍₆₎-tag (HHHHHH; SEQ ID NO:66) at the amino terminus of the protein to aid in purification, (ii) an E-tag (GAPVPYPDPLEPR; SEQ ID NO:67) at the carboxy terminus of the protein to aid in detection; (iii) an albumin-binding peptide (ABP) (DICLPRWGCLWD; SEQ ID NO:21) at the carboxyl end of the construct to increase serum half-life of the protein as V_{H}H monomers have a half-life of 2-3 hr and inclusion of ABP can increase the serum half-life to 10 hr (see FIG. 4); and a D7 tag (SSAPTKAKRRVVQREKT; SEQ ID NO: 112) at the carboxy terminus of the protein. The invention includes versions of the ABAB binding agent having one, two, three or four of these tags and peptides. An exemplary modified ABAB binding agent that includes the His tag and the D7 tag comprises the amino acid sequence set forth in SEQ ID NO:113 (the coding sequence is set forth in SEQ ID NO:114).

When yeast strains are engineered to produce ABAB, the protein can be also modified to include a secretion signal at the amino terminus of the protein. The secretion signal may be, but is not limited to, one of the sequences shown in Table 4.

TABLE 4
Secretion sequences for protein secretion in yeast

| Secretion signal | Amino acid sequence | Abbr. |
|---|---|---|
| α-factor_full | MRFPSIFTAVLFAASSALAAPVNTTTEDETAQIPAEAVIGYSD | FAKS |
| (S. cerevisiae) | LEGDFDVAVLPFSNSTNNGLLFINTTIASIAAKEEGVSLEKRE | |
| | AEA (SEQ ID NO: 96) | |
| α-factor_T_kex_ste | MRFPSIFTAVLFAASSALAAPVNTTTEDELEGDFDVAVLPFSA | AKS |
| (S. cerevisiae) | SIAAKEEGVSLEKREAEA (SEQ ID NO: 97) | |
| α-factor_T_kex | MRFPSIFTAVLFAASSALAAPVNTTTEDELEGDFDVAVLP | AK |
| (S. cerevisiae) | FSASIAAKEEGVSLEKR (SEQ ID NO: 98) | |
| α-factor_T | MRFPSIFTA VLFAASSALA (SEQ ID NO: 99) | AT |
| (S. cerevisiae) | | |
| Alpha-amylase | MVAWWSLFLYGLQVAAPALA (SEQ ID NO: 100) | A.A. |
| (Aspergillus niger) | | |
| Glucoamylase | MSFRSLLALSGLVCSGLA (SEQ ID NO: 101) | GA |
| (Aspergillus awamori) | | |
| Inulinase | MKLAYSLLLPLAGVSA (SEQ ID NO: 102) | IN |
| (Kluyveromyces maxianus) | | |
| Invertase | MLLQAFLFLLAGFAAKISA (SEQ ID NO: 103) | IVS |
| (S. cerevisiae) | | |
| Killer protein | MTKPTQVLVRSVSILFFITLLHLVVA (SEQ ID NO: 104) | KP |
| (S. cerevisiae) | | |
| Lysozyme | MLGKNDPMCLVLVLLGLTALLGICQG (SEQ ID NO: 105) | LZ |
| (Gallus gallus) | | |
| Serum albumin | MKWVTFISLLFLFSSAYS (SEQ ID NO: 106) | SA |
| (Homo sapiens) | | |

Exemplary modified ABAB binding agents that include an amino-terminal secretion signal include AT-ABAB and IVS-ABAB.

An exemplary modified ABAB binding agent that is expressed from a plasmid in yeast or bacteria includes the ABAB binding agent set forth in SEQ ID NO: 107, which is encoded by the polynucleotide sequence set forth in SEQ ID NO: 108.

An exemplary modified ABAB binding agent that is expressed in yeast after chromosomal integration includes the ABAB binding agent set forth in SEQ ID NO: 109, which is encoded by the polynucleotide sequence set forth in SEQ ID NO:110.

Each of the binding agents of the invention binds to TedA and/or TedB with specificity. In certain aspects of the invention, the binding agents exhibit TedA and/or TcdB neutralizing activity.

For the sake of clarity it can be noted that as used herein, “mono-specific”, “bi-specific”, “tri-specific”, “tetra-specific”, etc., mean the particular binding agent binds to 1, 2, 3, 4, etc., different epitopes, respectively. As used herein, “monomeric”, “dimeric”, “trimeric”, “tetrameric”, etc., mean that the particular binding agent has 1, 2, 3, 4, etc., separate V_{H}H peptide monomers that bind to the epitopes, respectively. Thus, a mono-specific, dimeric binding agent would display two V_{H}H peptide monomers that bind to the same epitope (e.g., a homodimer), and a bi-specific, dimeric binding agent would have two V_{H}H peptide monomers that bind to two different epitopes (e.g., a heterodimer). A tetra-specific, octameric binding agent has eight V_{H}H peptide monomers that recognize four different epitopes.

### V_{H}H-Fc

It is well known that chimeric Fc-fusion proteins have the potential of increasing the half-life of a protein in vivo. This strategy has been applied in several FDA approved drugs, such as Etanercept. A proof-of principle study has shown that single-chain antibodies can be correctly assembled and expressed by B cells of transgenic mice carrying a mini-Ig construct encoding a dromedary V_{H}H and the Fc domain of human IgG. Also EG2-Fc, a chimeric anti-EGFR/EGFRVIII V_{H}H, exhibited excellent tumor accumulation in vivo and has pharmacokinetic properties that could improve glioblastoma targeting.

The present invention includes binding agents comprising V_{H}H peptide monomers joined to antibody Fc domains (V_{H}H-Fc), where the binding agents bind TcdA and/or TcdB. In these Fc domain-based binding agents, one, two, three, four or more of the V_{H}H peptide monomers are joined to the hinge, C_{H}2 and C_{H}3 regions of the Fc domain of an antibody heavy chain. Thus, the peptide monomers replace the Fab regions of the antibody.

The V_{H}H peptide monomers may be any of those provided in Table 1 above and include 5D (SEQ ID NO:1), E3 (SEQ ID NO:3), AA6 (SEQ ID NO:5) and AH3 (SEQ ID NO:7) V_{H}H peptide monomers. Where two or more monomers are linked, the monomers may be linked by flexible peptide linkers, generally comprising between 10 and 20 amino acids. Suitable linkers include those linkers provided in Table 2, such as linker-1 (SEQ ID NO:9), linker-2 (SEQ ID NO: 11), and linker-3 (SEQ ID NO:13).

While the V_{H}H-Fc will typically be composed of two identical chains that self-assemble intracellularly after production, the invention also includes V_{H}H-Fc binding agents comprising two different Fc chains. In such circumstances, the sequence of the V_{H}H monomer(s) alone may differ between the two Fc chains, or the Fc chains themselves may differ in sequence, or both the V_{H}H monomer(s) and the Fc chains may differ in sequence.

One type of V_{H}H-Fc binding agent is an octameric binding agent comprising an antibody Fc domain and first, second, third and fourth V_{H}H peptide monomers, where the V_{H}H peptide monomers have binding specificity for an epitope of TedA or toxin B TcdB, where the first, second, third and fourth V_{H}H peptide monomers are linked together and joined to amino termini of both antibody Fc domains, and where the antibody Fc domain comprises the hinge, C_{H}2 and C_{H}3 regions of an antibody heavy chain. Because this binding agent has four V_{H}H peptide monomers, it can be mono-specific (where all of the monomers bind the same epitope), bi-specific (where the monomers bind two different epitopes), tri-specific (where the monomers bind three different epitopes), or tetra-specific (where the monomers bind four different epitopes).

A specific example of a tetra-specific V_{H}H-Fc binding agent is the ABAB-Fc binding agent, a tetra-specific, octameric binding agent comprising an antibody Fc domain and two sets of linked first, second, third and fourth V_{H}H peptide monomers, wherein the antibody Fc domain comprises two arms, each arm comprising hinge, C_{H}2 and C_{H}3 regions of an antibody heavy chain, and each arm having an amino terminus, wherein for each arm of the Fc domain, one set of linked first, second, third and fourth V_{H}H peptide monomers is joined to the amino terminus of the arm, and where the V_{H}H peptide monomers have binding specificity for an epitope of TcdA or TcdB (see FIG. 1). This binding agent is termed “tetra-specific” as it recognizes four different toxin epitopes. It is termed “octameric” as it bears eight V_{H}H peptide monomers (two copies of the first monomer, two copies of the second monomer, two copies of the third monomer, and two copies of the fourth monomer). ABAB-Fc was found to exhibit specific binding and neutralizing activity.

The ABAB-Fc binding agent was prepared by generating an expression vector encoding the V_{H}H peptide monomers AH3/5D/AA6/E3 (linked in the noted order) joined to a human IgG1 Fc domain. The V_{H}H peptide monomers were separated by flexible linkers of Table 2. The nucleic acid sequence encoding each chain is provided in SEQ ID NO:23. The amino acid sequence of each chain is provided in SEQ ID NO:22. Upon self-assembly of pairs of the chains after expression, the tetra-specific, octameric binding agent resulted. The invention includes the ABAB-Fc binding agent of SEQ ID NO:22, modified versions of ABAB binding agents as defined above, and sequence variants thereof having at least 85%, 86%, 87%, 88%, 89%, 90%, 91%, 92%, 93%, 94%, 95%, 96%, 97%, 98%, or 99% sequence identity over the entire length of the protein sequence and retaining the toxin binding and/or neutralizing activity of the wild-type protein. The present invention further includes polynucleotide sequences encoding these sequence variants and complementary strands thereof.

Mono-specific V_{H}H-Fc binding agents (AH3-Fc, 5D-Fc, E3-Fc, AA6-Fc) and bi-specific V_{H}H-Fc binding agents (e.g., AH3/5D-Fc and AA6/E3-Fc) were also made using this Fc-fusion system. With respect to mono-specific binding agents, single V_{H}H peptide monomers were joined to human IgG1 Fc domains. Upon expression and assembly, pairs of the chains resulted in mono-specific, dimeric binding agents (when the chains were identical) or bi-specific, dimeric binding agents (when the chains were different). With respect to bi-specific binding agents, two linked V_{H}H peptide monomers (V_{H}H homo- or hetero-dimers) were joined to human IgG1 Fc domains. Upon expression and assembly, pairs of the chains resulted in bi-specific, tetrameric binding agents (when the chains were identical) or tetra-specific, tetrameric binding agents (when the chains were different). Table 5 provides the sequences for some these binding agents.

TABLE 5

| | SEQ ID | SEQ ID |
|---|---|---|
| | NO for | NO for |
| | Amino | Nucleic |
| Name | Acid Seq. | Acid Seq. |
| 5D-Fc | 24 | 25 |
| E3-Fc | 26 | 27 |
| AA6-Fc | 28 | 29 |
| AH3-Fc | 30 | 31 |
| AH3-5D-Fc | 32 | 33 |
| AA6-E3-Fc | 34 | 35 |

Specific pairings with one monomer include: 5D-Fc+5D-Fc; E3-Fc+E3-Fc; AA6-Fc+AA6-Fc; AH3-Fc+AH3-Fc; 5D-Fc+E3-Fc; 5D-Fc+AA6-Fc; 5D-Fc+AH3-Fc; E3-Fc+AA6-Fc; E3-Fc+AH3-Fc; and AA6-Fc+AH3-Fc. Specific pairings with two monomers include: AH3-5D-Fc+AH3-5D-Fc; AA6-E3-Fc+AA6-E3-Fc; and AH3-5D-Fc+AA6-E3-Fc.

Bi-specific, tetrameric V_{H}H-Fc binding agents were produced comprising an antibody Fc domain and two sets of linked first and second V_{H}H peptide monomers, wherein the antibody Fc domain comprises two arms, each arm comprising hinge, C_{H}2 and C_{H}3 regions of an antibody heavy chain, and each arm having an amino terminus, wherein for each arm of the Fc domain, one set of linked first and second V_{H}H peptide monomers is joined to the amino terminus of the arm, and where the V_{H}H peptide monomers have binding specificity for an epitope of TcdA or TcdB. This binding agent is termed “bi-specific” as it recognizes two different toxin epitopes. It is termed “tetrameric” as it bears four V_{H}H peptide monomers (two copies of the first monomer, and two copies of the second monomer). The first and second V_{H}H peptide monomers may have binding specificity for the same or different epitopes. The V_{H}H peptide monomers may independently have binding specificity for an epitope in the glucosyltransferase domain, cysteine protease domain, translocation domain or receptor binding domain of TedA or TcdB.

A specific example of a bi-specific, tetrameric V_{H}H-Fc binding agent comprises the amino acid sequence set forth in SEQ ID NO:32 (AH3/5D-Fc). The invention also includes sequence variants thereof having at least 95% sequence identity, where the sequence variant retains toxin-neutralizing activity. The variant amino acids of the sequence variant may be located in framework regions of the V_{H}H peptide monomers.

A specific example of a bi-specific, tetrameric V_{H}H-Fc binding agent comprises the amino acid sequence set forth in SEQ ID NO:34 (AA6/E3-Fc). The invention also includes sequence variants thereof having at least 95% sequence identity, where the sequence variant retains toxin-neutralizing activity. The variant amino acids of the sequence variant may be located in framework regions of the V_{H}H peptide monomers.

The V_{H}H-Fc binding agents bind to TcdA and/or TedB with specificity. In certain aspects of the invention, the binding agents exhibit TcdA and/or TcdB neutralizing activity.

### V_{H}H-IgG

The present invention also includes binding agents comprising V_{H}H peptide monomers joined to more of an antibody that the Fc domain alone. V_{H}H-IgG binding agents comprise one, two, three, four or more of the V_{H}H peptide monomers are joined to the light (kappa or lambda) and heavy chains of an IgG antibody lacking the variable regions of the antibody. Thus, the peptide monomers replace the variable regions of the antibody.

The V_{H}H peptide monomers may be any of those provided in Table 1 above and include 5D (SEQ ID NO:1), E3 (SEQ ID NO:3), AA6 (SEQ ID NO:5) and AH3 (SEQ ID NO:7) V_{H}H peptide monomers. Where two or more monomers are linked, the monomers may be linked by flexible peptide linkers, generally comprising between 10 and 20 amino acids. Suitable linkers include those linkers provided in Table 2, such as linker-1 (SEQ ID NO:9), linker-2 (SEQ ID NO: 11), and linker-3 (SEQ ID NO:13).

V_{H}H-IgG binding agents include octameric binding agents comprising an IgG antibody and first, second, third and fourth V_{H}H peptide monomers, wherein the V_{H}H peptide monomers have binding specificity for an epitope of TedA or TcdB, wherein first and second V_{H}H peptide monomers are linked together and joined to amino termini of both light chains of the antibody, wherein the light chains lack the antibody variable regions, and wherein third and fourth V_{H}H peptide monomers are linked together and joined to amino termini of both heavy chains of the antibody, wherein the heavy chains lack the antibody variable regions. Because this binding agent has four V_{H}H peptide monomers, it can be mono-specific (where all of the monomers bind the same epitope), bi-specific (where the monomers bind two different epitopes), tri-specific (where the monomers bind three different epitopes), or tetra-specific (where the monomers bind four different epitopes).

A specific example of a tetra-specific V_{H}H-IgG binding agent is the ABAB-IgG binding agent, a tetra-specific, octameric binding agent comprising an IgG antibody, two sets of linked first and second V_{H}H peptide monomers, and two sets of linked third and fourth V_{H}H peptide monomers, wherein the IgG antibody comprises two arms, each arm comprising a heavy chain lacking a variable region and a light chain lacking a variable region, and each chain having an amino terminus, wherein for each arm of the antibody, one set of linked first and second V_{H}H peptide monomers is joined to the amino terminus of the light chain, and one set of linked third and fourth V_{H}H peptide monomers is joined to the amino terminus of the heavy chain, and wherein the V_{H}H peptide monomers have binding specificity for an epitope of TedA or TedB (see FIG. 1). This binding agent is termed “tetra-specific” as it recognizes four different toxin epitopes. It is termed “octameric” as it bears eight V_{H}H peptide monomers (two copies of the first monomer, two copies of the second monomer, two copies of the third monomer, and two copies of the fourth monomer). In certain aspects, the first, second, third and fourth V_{H}H peptide monomers may each have binding specificity for a different epitope. In certain aspects, two of the V_{H}H peptide monomers may have binding specificity for epitopes of TedA and two of the V_{H}H peptide monomers may have binding specificity for epitopes of TcdB. In certain aspects, the V_{H}H peptide monomers independently have binding specificity for an epitope in the glucosyltransferase domain, cysteine protease domain, translocation domain or receptor binding domain of TedA or TcdB.

A specific example of a tetra-specific, octameric ABAB-IgG binding agent comprises a light (kappa) chain having the amino acid sequence set forth in SEQ ID NO:46 (AA6/E3 kappa) or a sequence variant having at least 95% sequence identity thereto, and a heavy chain having the amino acid sequence set forth in SEQ ID NO:44 (AH3/5D heavy) or a sequence variant having at least 95% sequence identity thereto. In this aspect, the sequence variants retain toxin-neutralizing activity. The variant amino acids of the sequence variant may be located in framework regions of the V_{H}H peptide monomers. This binding agent was produced by preparing two separate expression vectors, the first encoding the V_{H}H peptide monomers AH3/5D (linked in the noted order) joined to the human IgG1 antibody heavy chain lacking the variable region and the second encoding the V_{H}H peptide monomers AA6/E3 (linked in the noted order) joined to the human IgG1 antibody light (kappa) chain lacking the variable region. The nucleotide sequence encoding the AA6/E3-IgG1 light (kappa) chain is provided in SEQ ID NO: 47. The nucleotide sequence encoding the AH3/5D-IgG1 heavy chain is provided in SEQ ID NO: 45. The invention includes sequence variants of ABAB-IgG having at least 85%, 86%, 87%, 88%, 89%, 90%, 91%, 92%, 93%, 94%, 95%, 96%, 97%, 98%, or 99% sequence identity over the entire length of the protein sequence and retaining the toxin binding and/or neutralizing activity of the wild-type protein. The present invention further includes polynucleotide sequences encoding these sequence variants and complementary strands thereof.

Bi-specific or tetra-specific, tetrameric IgG binding agents are included in the invention. Such binding agents comprise an IgG antibody and first, second, third and fourth V_{H}H peptide monomers, wherein the IgG antibody comprises two arms, each arm comprising a heavy chain lacking a variable region and a light chain lacking a variable region, and each chain having an amino terminus, wherein for a first arm of the antibody, the first V_{H}H peptide monomer is joined to the amino terminus of the light chain, and the second V_{H}H peptide monomer is joined to the amino terminus of the heavy chain, wherein for a second arm of the antibody, the third V_{H}H peptide monomer is joined to the amino terminus of the light chain, and the fourth V_{H}H peptide monomer is joined to the amino terminus of the heavy chain, and where the V_{H}H peptide monomers have binding specificity for an epitope of TedA or TcdB. When the binding agent is “tetra-specific”, it recognizes four different toxin epitopes; when “bi-specific” it recognizes two different toxin epitopes. The binding agents “tetrameric” as they bear four V_{H}H peptide monomers (when bi-specific, the first and second monomer have the same sequence and bind the same epitope, and the third and fourth monomers have the same sequence and bind the same epitope; when tetra-specific, each of the monomers has a different sequence and binds a different epitope).

When the binding agent is bi-specific, the first and third monomers have binding specificity for different epitopes, the first and second monomers have identical amino acid sequences, and the third and fourth monomers have identical amino acid sequences. In certain aspects, one of the V_{H}H peptide monomers has binding specificity for an epitope of TcdA and one of the V_{H}H peptide monomers has binding specificity for an epitope of TcdB.

When the binding agent is tetra-specific, each of the V_{H}H peptide monomers has binding specificity for a different epitope. In certain aspects, two of the V_{H}H peptide monomers have binding specificity for epitopes of TcdA and two of the V_{H}H peptide monomers have binding specificity for epitopes of TcdB.

In certain aspects, each of the V_{H}H peptide monomers has binding specificity for epitopes of TcdA. In other aspects, each of the V_{H}H peptide monomers has binding specificity for epitopes of TcdB.

In certain aspects, the V_{H}H peptide monomers independently have binding specificity for an epitope in the glucosyltransferase domain, cysteine protease domain, translocation domain or receptor binding domain of TedA or TcdB.

A specific example of a bi-specific, tetrameric IgG binding agent comprises a light (kappa) chain having the amino acid sequence set forth in SEQ ID NO:40 (AA6 kappa) and a heavy chain having the amino acid sequence set forth in SEQ ID NO:36 (AH3 heavy). The invention also includes sequence variants thereof having at least 95% sequence identity, where the sequence variant retains toxin neutralizing activity. The variant amino acids of the sequence variant may be located in framework regions of the V_{H}H peptide monomers.

Another specific example of a bi-specific, tetrameric IgG binding agent comprises a light (kappa) chain having the amino acid sequence set forth in SEQ ID NO:42 (E3 kappa) and a heavy chain having the amino acid sequence set forth in SEQ ID NO:38 (5D heavy). The invention also includes sequence variants thereof having at least 95% sequence identity, where the sequence variant retains toxin neutralizing activity. The variant amino acids of the sequence variant may be located in framework regions of the V_{H}H peptide monomers.

Table 6 provides the sequences used to generate bi- and tetra-specific V_{H}H-IgG binding agents. Other suitable pairings include (i) 5D-IgG1-heavy chain+AA6-light (kappa or lambda) chain, and (ii) AH3-IgG1-heavy chain+E3-light (kappa or lambda) chain.

TABLE 6

| | SEQ ID | SEQ ID |
|---|---|---|
| | NO for | NO for |
| | Amino | Nucleic |
| Name | Acid Seq. | Acid Seq. |
| AH3-IgG1 heavy chain | 36 | 37 |
| 5D-IgG1 heavy chain | 38 | 39 |
| AA6-IgG1 light (kappa) chain | 40 | 41 |
| E3-IgG1 light (kappa) chain | 42 | 43 |
| AH3/5D-IgG1 heavy chain | 44 | 45 |
| AA6/E3-IgG light (kappa) chain | 46 | 47 |

However, the present invention includes IgG1 heavy chains joined to any of AH3, 5D, AA6 and E3, and IgG1 light (kappa or lambda) chains joined to any of AH3, 5D, AA6 and E3. Further, all possible combinations of the heavy and light (kappa or lambda) chains are encompassed herein.

### Humanized Binding Agents

Due to their small size and the high degree of identity of their framework to the human V_{H} framework of family III, V_{H}H peptide monomers are expected to exhibit low immunogenicity when administered to humans. While the systemic application of small monovalent V_{H}H monomers seems to induce little, if any, neutralizing antibody responses, protein immunogenicity generally increases with size and complexity. Two major hurdles for repeated and/or long-term in vivo use of V_{H}H monomers are their likely short half-life and potential immunogenicity. To increase the valence and circulating half-life, V_{H}H monomers can be fused with human IgG and Fc domains as discussed herein. To address possible immunogenicity, the V_{H}H monomers can be humanized as needed without compromising their expression level, affinity, solubility, and stability. These strategies should result in good expression, stability, and solubility of humanized V_{H}H monomers (hV_{H}H monomers), while retaining the antigen specificity and affinity of the loop donor V_{H}H.

hV_{H}H monomers that gain highest identity to human V_{H} gene(s) and possess the highest binding/neutralizing activity are selected, after which they are transferred into the V_{H}H-multimers (e.g., ABAB), V_{H}H-Fc and V_{H}H-IgG constructs to generate fully humanized binding agents, such as fully humanized ABAB, ABAB-IgG and ABAB-Fc binding agents. The protein sequences of these humanized binding agents can be essentially identical to that of a human antibody variant, despite the non-human origin of some of its CDR segments that are responsible for the ability of the antibody to bind to its target antigen. Therefore, this strategy decreases the chance for potential immunogenicity in vivo and thus increase their safety and half-life in vivo.

The binding agents of the present invention thus encompasses humanized versions of each of the binding agents defined herein, comprising hV_{H}H peptide monomers.

### Epitope Binding Fragments

The binding agents of the invention include epitope binding fragments of each of the V_{H}H-Fc and V_{H}H-IgG binding agents defined herein. Because the V_{H}H-Fc and V_{H}H-IgG binding agents are comparable in structure to human IgG antibodies, where the variable regions are replace by the V_{H}H monomers, terms for human antibody fragments are also applicable to the such binding agents. The fragments include, but are not limited to, Fab fragments, F(ab′) 2 fragments, single chain Fv (scFv) antibodies, and fragments produced by an Fab expression library, as well as bi-specific antibody and triple-specific antibodies.

The V_{H}H-Fc and V_{H}H-IgG binding agents of the invention include fully human, humanized, and chimeric binding agents. The binding agents may be monoclonal or polyclonal. Further, the binding agents may be recombinant binding agents.

The binding agents may be produced in any species of animal, though preferably from a mammal such as a human, simian, mouse, rat, rabbit, guinea pig, horse, cow, sheep, goat, pig, dog or cat. For example, the binding agents can be human or humanized, or any binding agent preparation suitable for administration to a human.

### Polynucleotide, Expression Vectors, Host Cells and Method of Making

The invention includes polynucleotides comprising nucleotide sequences encoding each the binding agents provided herein, as well as complementary strands thereof.

The invention also includes expression vectors comprising the polynucleotides, and host cells comprising the expression vectors. Suitable expression vectors include, e.g., pcDNA3.1 and pSec-His, as well as plasmids used to transform yeast cells into producers and secretors of the binding agents of the invention. Suitable host cells include, e.g., Chinese hamster ovary cells (CHO cells), human embryonic kidney cells 293 (HEK 293 cells), yeast cells, and insect cells.

The invention further includes methods of producing the binding agents defined herein, comprising culturing the host cells under conditions promoting expression of the binding agents encoded by the expression vectors, and recovering the binding agents from the cell cultures.

### Engineered Strains of Yeast

Each of the binding agents of the invention may also be produced by engineered strains of Saccharomyces yeast. Accordingly, the invention is also directed to strains of Saccharomyces yeast, such as S. cerevisiae and S. boulardii, engineered to produce one or more of the binding agents defined herein including, but not limited to, V_{H}H monomer binding agents (see Table 1), V_{H}H homodimer binding agents, V_{H}H heterodimer binding agents (see Table 3), ABAB binding agents, V_{H}H-Fc binding agents (see Table 5), V_{H}H-IgG binding agents (see Table 6), and epitope biding fragments thereof. In preferred aspects, the engineered strains of Saccharomyces yeast secrete the binding agents.

The identity of the Saccharomyces yeast strain is only limited in that it can be engineered to produce, and preferably secrete, one or more of the binding agents of the invention. In preferred aspects of the invention, the strain of Saccharomyces yeast engineered to produce one or more of the binding agents is S. cerevisiae or S. boulardii. The invention thus encompasses an engineered strain of S. cerevisiae that produces one or more of the binding agents defined herein, as well as an engineered strain of S. cerevisiae that secretes one or more of the binding agents defined herein. The invention also encompasses an engineered strain of S. boulardii that produces one or more of the binding agents defined herein, as well as an engineered strain of S. boulardii that secretes one or more of the binding agents defined herein. Suitable stains of yeast also include Schizosaccharomyces pombe, Saccharomyces paradoxus, and Saccharomyces unisporus.

S. boulardii is an FDA-designated Generally Regarded as Safe (GRAS) organism and it is commonly available over-the-counter for use in promoting intestinal health and amelioration of gastrointestinal illness due to diarrheal diseases. This species of yeast has been studied in multiple randomized double-blinded placebo-controlled clinical trials for both safety and efficacy against intestinal diseases including CDI [42-46]. A suitable strain of S. boulardii is the S. boulardii strain MYA796 (ATCC, Manassas, VA).

A particular example of the engineered strains of Saccharomyces yeast of the invention is an engineered strain of Saccharomyces yeast that produces a binding agent comprising a V_{H}H peptide monomer or linked groups of V_{H}H peptide monomers comprising two, three, four, or more monomers, each of which binds TcdA and/or TcdB, preferably with specificity. Thus, the invention encompasses engineered strains of Saccharomyces yeast that produces V_{H}H peptide binding agents comprising at least one V_{H}H peptide monomer, wherein each V_{H}H peptide monomer has binding specificity for an epitope of C. difficile toxin A (TcdA) or toxin B (TcdB). In certain aspects, these binding agents comprise two, three, four, or more linked V_{H}H peptide monomers. The V_{H}H peptide monomers include, but are not limited to, the V_{H}H peptide monomers 5D (SEQ ID NO:1), E3 (SEQ ID NO:3), AA6 (SEQ ID NO:5), and AH3 (SEQ ID NO:7).

Another particular example of the engineered strains of Saccharomyces yeast of the invention is an engineered strain of Saccharomyces yeast that produces binding agents comprising V_{H}H peptide monomers joined to IgG antibodies, where the binding agents bind TcdA and/or TcdB, as defined herein. In these IgG-based binding agents, the variable regions of the light and heavy chains of IgG antibodies are replaced by one, two, three, four or more of the V_{H}H peptide monomers.

A further particular example of the engineered strains of Saccharomyces yeast of the invention is an engineered strain of Saccharomyces yeast that produces binding agents comprising V_{H}H peptide monomers joined to antibody Fc domains, where the binding agents bind TcdA and/or TcdB, as defined herein. In these Fc domain-based binding agents, one, two, three, four or more of the V_{H}H peptide monomers are joined to the hinge, C_{H}2 and C_{H}3 regions of each arm of Fc domain of an antibody heavy chain. Thus, the peptide monomers replace the Fab regions of an antibody.

An additional particular example of the engineered strains of Saccharomyces yeast of the invention is an engineered strain of Saccharomyces yeast that produces a tetra-specific, tetrameric binding agent, wherein the binding agent comprises linked first, second, third and fourth V_{H}H peptide monomers, and wherein the V_{H}H peptide monomers independently have binding specificity for an epitope of Clostridium difficile toxin A (TcdA) or toxin B (TcdB). In certain aspects, the first, second, third and fourth V_{H}H peptide monomers each has binding specificity for a different epitope. In certain aspects, the two of the V_{H}H peptide monomers have binding specificity for epitopes of TedA and two of the V_{H}H peptide monomers have binding specificity for epitopes of TcdB. In certain aspects, the V_{H}H peptide monomers independently have binding specificity for an epitope in the glucosyltransferase domain, cysteine protease domain, translocation domain or receptor binding domain of TedA or TcdB. Suitable V_{H}H peptide monomers include the AH3 monomer (SEQ ID NO:7), the AA6 monomer (SEQ ID NO: 5), the 5D monomer (SEQ ID NO:1), and the E3 monomer (SEQ ID NO:3). Other monomers include, but are not limited to, those provided in Table 1.

In a preferred example, the invention is directed to an engineered strain of yeast, wherein the binding agent is ABAB, wherein the first and third monomers have binding specificity for epitopes of TcdA and the first and third monomers are V_{H}H peptide monomers AH3 (SEQ ID NO:7) and AA6 (SEQ ID NO:5), respectively, and wherein the second and forth monomers have binding specificity for epitopes of TcdB and the second and forth monomers are V_{H}H peptide monomers 5D (SEQ ID NO:1) and E3 (SEQ ID NO:3), respectively.

The ABAB binding agent may comprise the amino acid sequence set forth in SEQ ID NO: 19, or a sequence variant having at least 95% sequence identity thereto, wherein the sequence variant retains TcdA and/or TcdB binding specificity, or the sequence variant retains toxin neutralizing activity, or both.

The ABAB binding agent may also comprises an N-terminal secretion signal selected from the secretion signals provided in Table 4. In preferred aspects, the N-terminal secretion signal is the AT secretion signal (MRFPSIFTAVLFAASSALA (SEQ ID NO:99)) or the IVS secretion signal (MLLQAFLFLLAGFAAKISA (SEQ ID NO:103)).

The ABAB binding agent may be expressed from a plasmid within the yeast. The plasmid may be, but is not limited to, pCEV-URA3-TEF-AT-yABAB-cMyc (SEQ ID NO:88). The ABAB binding agent encoded by the plasmid may comprises the amino acid sequence set forth in SEQ ID NO:107, or a sequence variant having at least 95% sequence identity thereto, and wherein the sequence variant retains TcdA and/or TcdB binding specificity, or the sequence variant retains toxin neutralizing activity, or both.

The ABAB binding agent may also be expressed from coding sequence integrated into a chromosome of yeast. The ABAB binding agent expressed from coding sequence integrated into a yeast chromosome may comprises the amino acid sequence set forth in SEQ ID NO: 109, or a sequence variant having at least 95% sequence identity thereto, and wherein the sequence variant retains TcdA and/or TedB binding specificity, or the sequence variant retains toxin neutralizing activity, or both.

The invention is also directed to engineered strains of Saccharomyces yeast that produce a therapeutic protein having binding specificity for a unique epitope of Clostridium difficile toxin A (TcdA) or toxin B (TcdB), or both. Preferably, the engineered strain of Saccharomyces yeast is S. cerevisiae or S. boulardii. A therapeutic protein is any protein that can bring about an improvement or cure in a medical condition in a subject, or that can inhibit or prevent a medical condition from developing in a subject. Suitable therapeutic protein include, but are not limited to, proteins that (a) replace a protein that is deficient or abnormal; (b) augment an existing pathway; (c) provide a novel function or activity; (d) interfere with a molecule or organism; and (e) deliver other compounds or proteins, such as a radionuclide, cytotoxic drug, or effector proteins. Therapeutic proteins also include antibodies and antibody-based drugs, Fc fusion proteins, anticoagulants, blood factors, bone morphogenetic proteins, engineered protein scaffolds, enzymes, growth factors, hormones, interferons, interleukins, and thrombolytics. Therapeutic proteins further include bispecific monoclonal antibodies (mAbs) and multispecific fusion proteins, mAbs conjugated with small molecule drugs, and proteins with optimized pharmacokinetics.

### Methods of Making Engineered Yeast Strains

The invention is also directed to methods of engineering strains of Saccharomyces yeast to produce one or more of the binding agents defined herein. The means used to produce the engineered strains of yeast are not particularly limited and there are a number of well-established techniques available for engineering yeast to produce homologous and heterologous proteins that will be known to the skilled artisan. In certain aspects of these methods, S. cerevisiae or S. boulardii is engineered to produce the binding agents.

As an example, Saccharomyces yeast may be engineered to produce one or more of the binding agents defined herein by (a) transforming a strain of Saccharomyces yeast with an expression vector encoding the binding agent, and (b) screening the resulting yeast for production of the binding agent. In a certain aspect, the expression vector is plasmid pCEV-URA3-TEF-AT-yABAB-cMyc (SEQ ID NO:88). While this plasmid encodes a particular ABAB binding agent, the coding region for this binding agent can be replaced by the coding region of any of the binding agents defined herein.

As a further example, Saccharomyces yeast may be engineered to produce one or more of the binding agents defined herein by (a) chromosomally integrating a polynucleotide sequence encoding the binding agent into the genome of the strain of Saccharomyces yeast, and (b) screening the yeast of (a) for production of the binding agent. In certain aspects, the chromosomal integration is performed using a C^{RISPR} technique [85-88]. As an example, such a method may include the steps of: (a) amplifying a polynucleotide sequence encoding the ABAB binding agent from plasmid pCEV-G4-Km-TEF-AT-yABAB hAA6T83N-tagless (SEQ ID NO: 90) using primers containing (i) nucleic acid sequence homologous to a selected yeast chromosomal integration site and (ii) nucleic acid sequence homologous to regions 5′ and 3′ of ABAB binding agent coding sequence of the plasmid, to produce an integration cassette, (b) transforming yeast with the integration cassette produced in (a) with pCRI-Sb-81 (SEQ ID NO: 91) or pCRI-Sb-82 (SEQ ID NO:92) to induce a double stranded break within the corresponding yeast chromosomal delta sites under conditions promoting spontaneous integration of the integration cassette into the site of the double stranded break, (c) screening the transformed yeast of (b) for production of the ABAB binding agent.

While the plasmid pCEV-G4-Km-TEF-AT-yABAB hAA6T83N-tagless encodes a particular ABAB binding agent, the coding region for this binding agent can be replaced by the coding region of any of the binding agents defined herein.

Suitable means used to screen the yeast for production of the binding agents will be readily apparent to the skilled artisan and include, but are not limited to immunoassays, such as an ELISA or a western blot.

### Methods of Treatment and Prevention

The binding agents and engineered strains of Saccharomyces yeast of the invention can be used in methods of treating or preventing a disease symptom induced by C. difficile in a subject. These methods generally comprise administering a therapeutically-effective amount of one or more binding agents and/or one or more engineered strains of Saccharomyces yeast as defined herein to a subject having C. difficile infection or a risk of developing C. difficile infection. In certain aspects of this embodiment, the disease symptom induced by C. difficile is diarrhea

The binding agents and engineered strains of Saccharomyces yeast of the invention can also be used in of neutralizing C. difficile toxin TedA and/or TedB in a subject infected by C. difficile. These methods generally comprise administering a therapeutically-effective amount of one or more binding agents and/or one or more engineered strains of Saccharomyces yeast as defined herein to a subject having C. difficile infection.

The binding agents and engineered strains of Saccharomyces yeast of the invention can further be used in methods of treating C. difficile infection in a subject. These methods generally comprise administering a therapeutically-effective amount of one or more of the binding agents and/or one or more engineered strains of Saccharomyces yeast as defined herein to a subject having C. difficile infection. These same methods can be used to treat CDI, as defined herein.

The binding agents and engineered strains of Saccharomyces yeast of the invention can also be used in methods of maintaining normal bowel function in a subject having a C. difficile infection. These methods generally comprise administering a therapeutically-effective amount of one or more of the binding agents and/or one or more engineered strains of Saccharomyces yeast as defined herein to a subject having C. difficile infection or a risk of developing C. difficile infection.

The binding agents and engineered strains of Saccharomyces yeast can also be used in immunoprophylaxis in order to prevent immediate CDI threats. In addition, passive immunoprophylaxis can be used to prevent both immediate and longer-term CDI threats. Each approach has its own particular advantages and is suitable to target a particular high-risk population. These methods generally comprises administering a therapeutically-effective amount of one or more of the binding agent and/or one or more engineered strains of Saccharomyces yeast as defined herein to a subject a risk of developing C. difficile infection.

In preferred aspects of the methods of the invention, the Saccharomyces yeast is S. cerevisiae or S. boulardii.

Each of the methods of the invention may include administration of the one or more binding agents and/or the one or more engineered strains of Saccharomyces yeast in one or more pharmaceutical formulations comprising the binding agents and/or the engineered strains of Saccharomyces yeast and a pharmaceutically acceptable carrier or diluent. In preferred aspects, the Saccharomyces yeast is S. cerevisiae or S. boulardii.

As used herein, the terms “treat”, “treating”, and “treatment” have their ordinary and customary meanings, and include one or more of: blocking, ameliorating, or decreasing in severity and/or frequency a symptom of a C. difficile infection or a C. difficile-related disease (CDI) in a subject; and/or partly or fully inhibiting the biological activity and/or promoting the immunologic clearance of C. difficile TcdA and/or TedB in a subject infected with C. difficile; and/or growth, division, spread, or proliferation of C. difficile cells or a C. difficile infection in a subject. Treatment means blocking, ameliorating, decreasing, or inhibiting by about 1% to about 100% versus a subject in which the methods of the present invention have not been practiced. Preferably, the blocking, ameliorating, decreasing, or inhibiting is about 100%, 99%, 98%, 97%, 96%, 95%, 90%, 80%, 70%, 60%, 50%, 40%, 30%, 20%, 10%, 5% or 1% versus a subject in which the methods of the present invention have not been practiced.

As used herein, the terms “prevent”, “preventing” and “prevention” have their ordinary and customary meanings, and include one or more of, stopping, averting, avoiding, alleviating or blocking C. difficile from colonizing, developing or progressing in a subject; and/or partly or fully inhibiting the biological activity and/or toxic effects of TedA and/or TcdB in a subject infected with C. difficile; and/or stopping, averting, avoiding, alleviating or blocking the growth, division, spread, or proliferation of bacterial cells or bacterial infection in a subject. Prevention means stopping by at least about 95% versus a subject to which the prevention has not been administered. Preferably, the stopping is about 100%, about 99%, about 98%, about 97%, about 96% or about 95%. The results of the prevention may continue for a period of days (such as 1, 2, 3, 4, 5, 6 or 7 days), weeks (such as 1, 2, 3 or 4 weeks) or months (such as 1, 2, 3, 4, 5, 6 or more months).

The method of treating and preventing provided herein can be supplemented by also administering a therapeutically-effective amount of an antibiotic to the subject. Preferably, the antibiotic will have antibacterial activity against C. difficile.

### Pharmaceutical Formulations

While the binding agents and engineered strains of Saccharomyces yeast may be administered directly to a subject, the methods of the present invention are preferably based on the administration of a pharmaceutical formulation comprising one or more binding agents and/or one or more engineered strains of Saccharomyces yeast, and a pharmaceutically acceptable carrier or diluent. Thus, the invention includes pharmaceutical formulations comprising one or more of the binding agents and/or one or more engineered strains of Saccharomyces yeast defined herein and a pharmaceutically acceptable carrier or diluent.

Pharmaceutically acceptable carriers and diluents are commonly known and will vary depending on the particular binding agent or engineered strains of Saccharomyces yeast being administered and the mode of administration. Examples of generally used carriers and diluents include, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents. The formulations comprising binding agents and/or engineered strains of Saccharomyces yeast will typically have been prepared and cultured in the absence of any non-human components, such as animal serum (e.g., bovine serum albumin).

Pharmaceutical formulations comprising one or more binding agents and/or one or more engineered strains of Saccharomyces yeast may be administered to a subject using modes and techniques known to the skilled artisan. Characteristic of CDI disease may make it more amenable to treatment and prevention using colonic delivery of therapeutic agents, i.e., targeted delivery of binding agents to the lower GI tract, e.g., the large intestine or colon. Other modes of delivery include, but are not limited to, oral, nasal, anal, and via intravenous injection or aerosol administration. Other modes include, without limitation, intradermal, subcutaneous (s.c., s.q., sub-Q, Hypo), intramuscular (i.m.), intraperitoneal (i.p.), intra-arterial, intramedulary, intracardiac, intra-articular (joint), intrasynovial (joint fluid area), intracranial, intraspinal, and intrathecal (spinal fluids).

Depending on the means of administration, the dosage may be administered all at once, such as with an oral formulation in a capsule or liquid, or slowly over a period of time, such as with an intramuscular or intravenous administration.

The amount of binding agents, alone or in a pharmaceutical formulation, administered to a subject is an amount effective for the treatment or prevention of infection. Thus, therapeutically effective amounts are administered to subjects when the methods of the present invention are practiced. In general, between about 1 μg/kg and about 1000 mg/kg of the binding agent per body weight of the subject is administered. Suitable ranges also include between about 50 μg/kg and about 500 mg/kg, and between about 10 μg/kg and about 100 mg/kg. However, the amount of binding agent administered to a subject will vary between wide limits, depending upon the location, source, extent and severity of the infection, the age and condition of the subject to be treated, the means of administration, etc. A physician will ultimately determine appropriate dosages to be used.

The amount of the engineered strains of Saccharomyces yeast, alone or in a pharmaceutical formulation, administered to a subject is an amount effective for the treatment or prevention of infection. Thus, therapeutically effective amounts are administered to subjects when the methods of the present invention are practiced. In general, between about 1 μg/kg and about 1000 mg/kg of the engineered strains of Saccharomyces yeast per body weight of the subject is administered. Suitable ranges also include between about 50 μg/kg and about 500 mg/kg, and between about 10 μg/kg and about 100 mg/kg. However, the amount of the engineered strains of Saccharomyces yeast administered to a subject will vary between wide limits, depending upon the location, source, extent and severity of the infection, the age and condition of the subject to be treated, the means of administration, etc. A physician will ultimately determine appropriate dosages to be used.

Administration frequencies of the binding agents, the engineered strains of Saccharomyces yeast, and pharmaceutical formulations comprising the binding agents and/or engineered strains of Saccharomyces yeast will vary depending on factors that include the location of the bacterial infection, the particulars of the infection to be treated or prevented, and the mode of administration. Each formulation may be independently administered 4, 3, 2 or once daily, every other day, every third day, every fourth day, every fifth day, every sixth day, once weekly, every eight days, every nine days, every ten days, bi-weekly, monthly and bi-monthly.

The duration of treatment or prevention will be based on location and severity of the infection being treated or the relative risk of contracting the infection, and will be best determined by the attending physician. However, continuation of treatment is contemplated to last for a number of days, weeks, or months.

In each embodiment and aspect of the invention, the subject is a human, a non-human primate, bird, horse, cow, goat, sheep, a companion animal, such as a dog, cat or rodent, or other mammal. The subjects to which the methods of the present invention can be applied include subjects having an underlying disease or condition that makes them more susceptible to C. difficile infections.

The invention also provides a kit comprising one or more containers filled with one or more of the binding agents, one or more of the engineered strains of Saccharomyces yeast, or one or more pharmaceutical formulations comprising binding agents and/or the engineered strains of Saccharomyces yeast. The kit may also include instructions for use. Associated with the kit may further be a notice in the form prescribed by a governmental agency regulating the manufacture, use or sale of pharmaceuticals or biological products, which notice reflects approval by the agency of manufacture, use or sale for human administration.

### III. Examples

### V_{H}H Monomer and Heterodimer Binding Agents

An efficient platform to screen single domain (monomeric), mono-specific V_{H}H peptide monomers against specific domains of toxins TcdA and TcdB was established. Using highly immunogenic atoxic holotoxins for immunization, and bioactive chimeric toxins (with normal domain functions) for screening, panels of V_{H}H monomers binding to different domains of TcdA or TcdB were prepared. A majority of these V_{H}H monomers possessed potent neutralizing activity and their binding to specific domains was determined (FIG. 2). The atoxic holotoxins have point mutations at their enzymatic glucosyltransferase domains as described previously [33]. The bioactive chimeric toxins were created by switching the functional domains between TcdA and TcdB, which was also described previously [33].

Several of the V_{H}H monomers bind to highly conserved TcdA/TcdB epitopes. For example, V_{H}H E3 binds to the Rho GTPase binding site and blocks glucosylation; V_{H}H AH3 binds to the GT domain of the toxin; V_{H}H 7F binds to cysteine protease cleavage sites and blocks GT domain cleavage and release. Some V_{H}H monomers have potent neutralizing activity capable of blocking toxin cytotoxic activity at nM concentrations (See Table 1; FIGS. 3A and 3B).

To enhance the binding activity, two domain (dimeric), bi-specific V_{H}H heterodimers were created (Table 3; FIG. 3C), allowing a single protein to target two distinctive epitopes of the toxins. These bi-specific V_{H}H heterodimers possessed substantially enhanced neutralizing activities compared with equimolar mixtures of the same two V_{H}H monomers (FIG. 3D). Heterodimers 5D/E3 and AH3/AA6 were found to fully protect mice from lethal systemic TcdB or TcdA challenge respectively, whereas mixed 5D and E3, or AA6 alone were only partially protective (FIGS. 3E and 3F).

A tetra-valent, tri-specific V_{H}H binding agent (ABA) was generated by genetically fusing V_{H}Hs with the highest neutralizing activities targeting conserved, non-overlapping

epitopes (AH3/E3/E3/AA6) [41]. This rationally designed toxin binder achieved a substantially enhancing binding affinity and neutralizing activity over the individual monomers and potent therapeutic efficacy against fulminant CDI. ABA was able to broadly neutralize toxins from 11 different TcdA⁺TcdB⁺C. difficile clinical isolates but failed to neutralize TcdB derived from two TcdA⁻TcdB⁺ strains. The amino acid sequence of ABA is set forth in SEQ ID NO: 111.

The V_{H}H monomers comprising the heterodimers were linked using a flexible linker selected from SEQ ID NOs: 9-13 (Table 2).

### ABAB Binding Agent

Four domain (tetrameric), tetra-specific V_{H}H binding agents were generated by linking V_{H}H monomers AH3, 5D, E3, and AA6, namely ABBA (AH3/5D/E3/AA6) and ABAB (AH3/5D/AA6/E3). These tetra-specific, tetrameric binding agent targets conserved, non-overlapping epitopes and had excellent toxin neutralizing activity. In the design of ABAB (FIG. 4), V_{H}H peptide monomers AH3 and AA6 were separated by placing the 5D monomers between them because AH3 and AA6 bind to GT and TD respectively (FIG. 2), which are spatially distant to each other. This design allowed AH3 and AA6 to bind to TcdA simultaneously.

In the construction of the ABAB binding agent, flexible linkers were placed between the V_{H}H monomers (see FIG. 4). The complete nucleic acid sequence encoding ABAB is provided in SEQ ID NO:20; the amino acid sequence of the protein is provided in SEQ ID NO: 19.

In certain variants, a His₍₆₎-tag was provided at the amino terminus of the protein to aid in purification, an E-tag was provided at the carboxy terminus of the protein to aid in detection, and/or an albumin-binding peptide (ABP, DICLPRWGCLWD; SEQ ID NO:21) was placed at the carboxyl end of the construct to increase serum half-life of the protein (See FIG. 4).

ABAB was found to exhibit substantial enhanced binding affinity (Table 7) and neutralizing activity (Table 8) over the individual monomers and ABA. In Table 8, Vero cells were exposed to 5 ng/ml of TedA in the presence of serially diluted AA6, AH3, ABAB or Merck anti-TcdA HuMab [9]. The minimal doses of antibodies protecting cells from TcdA-induced cell rounding are shown.

TABLE 7

| | | Kₒₙ | K_{off} | K_{D} |
|---|---|---|---|---|
| | V_{H}Hs | (Ms⁻¹) | (s⁻¹) | (nM) |
| TcdA | AH3 | 2.20 × 10⁴ | 7.10 × 10⁻⁴ | 32.0 |
| | AA6 | 3.52 × 10⁴ | 6.92 × 10⁻⁴ | 19.7 |
| | ABAB | 6.96 × 10⁵ | 1.21 × 10⁻⁶ | 0.002 |
| TcdB | 5D | 1.52 × 10⁶ | 9.94 × 10⁻⁴ | 0.65 |
| | E3 | 2.95 × 10⁶ | 9.4 × 10⁻⁵ | 0.03 |
| | ABAB | 1.79 × 10⁶ | 3.57 × 10⁻⁶ | 0.002 |

TABLE 8

| | | | Merck |
|---|---|---|---|
| | | | Anti-TcdA |
| AA6 | AH3 | ABAB | HuMab |
| 8 nM | 8 nM | 0.25 nM | >10 nM |

ABAB was also found to compete with all four individual V_{H}H peptide monomers in a competition ELISA and can simultaneously bind to both TcdA and TcdB as determined by sandwich ELISA. Furthermore, ABAB is broadly reactive, capable of neutralizing toxins from the 13 different C. difficile strains that represent most of the current epidemic strains (Table 9).

TABLE 9

| | | | | | | ABAB |
|---|---|---|---|---|---|---|
| | Ribo- | REA | PFGE | | Place/date of | neutra- |
| Strains | type | type | type | Toxins | isolation | lization |
| R20291 | 27 | Bl | NAP1 | TcdA/Tcd | London/2006 | Yes |
| | | | | B | | |
| CD196 | 27 | Bl | NAP1 | TcdA/Tcd | France/1985 | Yes |
| | | | | B | | |
| 630 | 12 | R | | TcdA/Tcd | Zurich/1982 | Yes |
| | | | | B | | |
| M120 | 78 | BK | NAP7, 8, | TcdA/Tcd | UK/2007 | Yes |
| | | | 9 | B | | |
| BI-9 | 1 | J | NAP2 | TcdA/Tcd | Gerding | Yes |
| | | | | B | Collection | |
| Liv024 | 1 | J | NAP2 | TcdA/Tcd | Liverpool/2009 | Yes |
| | | | | B | | |
| Liv022 | 106 | DH | NAP11 | TcdA/Tcd | Liverpool/2009 | Yes |
| | | | | B | | |
| TL178 | 2 | G | NAP6 | TcdA/Tcd | Belfast/2009 | Yes |
| | | | | B | | |
| TL176 | 14 | Y | NAP4 | TcdA/Tcd | Cambridge, | Yes |
| | | | | B | UK/2009 | |
| TL174 | 15 | | | TcdA/Tcd | Cambridge, | Yes |
| | | | | B | UK/2009 | |
| CD305 | 23 | | | TcdA/Tcd | London/2008 | Yes |
| | | | | B | | |
| CFS | 17 | | | TcdB | Belgium/1995/ | Yes |
| | | | | | human | |
| M68 | 17 | | | TcdB | Dublin/2006/ | Yes |
| | | | | | human | |

Since ABAB shows high potency in binding to and neutralizing both toxins, its efficacy in treating fulminant CDI was evaluated. A single injection with as low as 40 μg/kg of ABAB one-day post C. difficile spore challenge reversed fulminant CDI in mice. None of the ABAB-treated mice died whereas 50% of control mice became moribund by 3 days post-infection (FIG. 5A). ABAB is 4-log more potent in preventing mortality after systemic challenge with TcdA and TcdB than the Merck HuMabs (FIG. 5B) [9]. Thus, ABAB possesses extraordinary in vivo efficacy against C. difficile toxins and spore challenge.

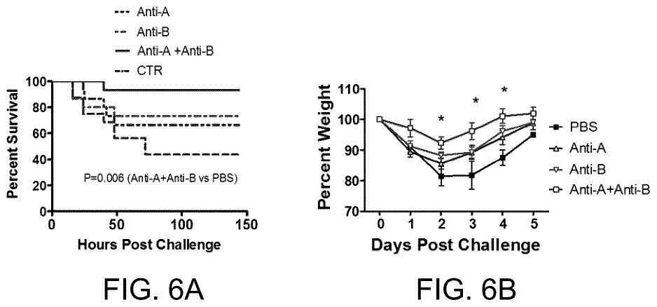

Animal and human studies demonstrated that passively administered antitoxin antibodies provide protection against CDI. The initial studies here also showed that antitoxin polysera protected mice from primary CDI (FIGS. 6A and 6B) and recurrent/relapse CDI. These findings and results from FIGS. 5A and 5B supported the hypothesis and provided the rationale for development of a parenteral ABAB immunization strategy for preventing CDI. To achieve the goal of optimizing ABAB for systemic delivery, chimeric and humanized ABAB were generated as illustrated in FIG. 1, i.e., V_{H}H-Fc and V_{H}H-IgG binding agents as well as the humanized proteins hV_{H}H-Fc and hV_{H}H-IgG, after which leading proteins were evaluated for in vivo neutralizing activity and protection in animal models. Details regarding the preparation and testing of the additional binding agents are provided in the following pararaphs.

### ABAB-Fc

ABAB-Fc binding agent was prepared by generating an expression vector encoding the V_{H}H peptide monomers AH3/5D/AA6/E3 (linked in the noted order) joined to a human IgG1 Fc domain. The V_{H}H peptide monomers were separated by flexible linkers of Table 2. The nucleic acid sequence encoding the protein is provided in SEQ ID NO:23. ABAB-Fc was expressed and purified from stable transfected HEK293 cell line culture supernatant using protein A beads under conditions permitting disulfide bond formation and bi-valent molecule production. The expression levels were about 20 mg/L of culture supernatant. ABAB-Fc is fully functional in binding and neutralizing both TcdA and TcdB (data not shown). The amino acid sequence of ABAB-Fc is provided in SEQ ID NO:22.

Mono-specific V_{H}H-Fc binding agents (AH3-Fc, 5D-Fc, E3-Fc, AA6-Fc) and bi-specific V_{H}H-Fc binding agents (AH3/5D-Fc) and AA6/E3-Fc) were also made using this Fc-fusion system. Table 5 above provides the sequences for these additional binding agents.

### ABAB-IgG

As illustrated in FIG. 1, bi-specific V_{H}H-IgG (AH3/5D-IgG and E3/AA6-IgG) can be generated by fusing monomers with human IgG heavy and light (kappa or lambda) chains separately. Tetra-specific V_{H}H-IgG (ABAB-IgG) binding agents can be generated by fusing dimers with human IgG heavy and light chains separately. Co-transfecting the heavy and light chain constructs generates the AH3/5D-IgG, E3/AA6-IgG and ABAB-IgG chimeric proteins. The separation of two V_{H}Hs into heavy and light chains likely improves the yield and stability of bi-specific and tetra-specific V_{H}H chimeric proteins. This allows determination of whether V_{H}H-human IgG chimeric antibody helps the stability and efficacy of ABAB in vivo. Similarly, further improvement of in vivo half-life of ABAB-IgG can also be tested in ABAB-IgG variants with enhanced binding affinity to FcRn receptor.

Bi-specific (AH3/5D-IgG1 and E3/AA6-IgG1) and tetra-specific (ABAB-IgG1) IgG1 binding agents were prepared by co-transfecting expression vectors encoding the heavy and light (kappa) chain of each binding agent. The V_{H}H peptide monomers were separated by flexible linkers of Table 2.

Bi-specific, tetrameric V_{H}H-IgG1 binding agents were produced by preparing two separate expression vectors, the first encoding a V_{H}H peptide monomer joined to the human IgG1 antibody heavy chain (C_{H}1-Hinge-C_{H}2-C_{H}3) lacking the heavy chain variable region and the second encoding a V_{H}H peptide monomer joined to the human IgG1 antibody light (kappa) chain (CK) lacking the light chain variable region. These binding agents are bi-specific and tetrameric in that each light chain of the resulting binding agent is linked to a first V_{H}H monomer and each heavy chain of the resulting binding agent is linked to a second V_{H}H monomer. Table 6 above provides the sequences for these additional binding agents. Suitable pairings include (i) AH3-IgG1-heavy chain+AA6-light (kappa or lambda) chain, (ii) 5D-IgG1-heavy chain+E3-light (kappa or lambda) chain, (iii) 5D-IgG1-heavy chain+AA6-light (kappa or lambda) chain, and (iv) AH3-IgG1-heavy chain+E3-light (kappa or lambda) chain.

Tetra-specific, octameric ABAB-IgG binding agents were prepared. These binding agents are tetra-specific and octameric in that each light (kappa or lambda) chain of the resulting binding agent is joined to two (a first and second) linked V_{H}H monomers and each heavy chain of the resulting binding agent is joined to a two (a third and fourth) linked V_{H}H monomer, where the first, second, third and fourth monomers binds to a different epitope.

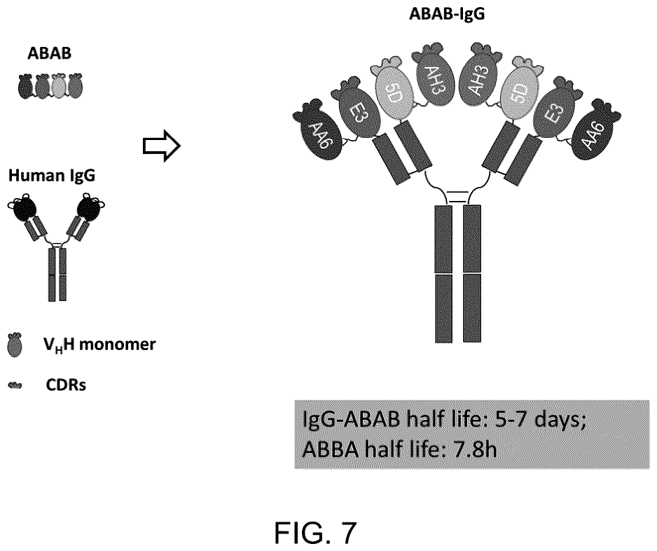

A particular tetra-specific, octameric ABAB-IgG (FIG. 7) binding agent was produced by preparing two separate expression vectors, the first encoding the V_{H}H peptide monomers AH3/5D (linked in the noted order) joined to the human IgG1 antibody heavy chain (C_{H}1-Hinge-C_{H}2-C_{H}3) lacking the heavy chain variable region and the second encoding the V_{H}H peptide monomers AA6/E3 (linked in the noted order) joined to the human IgG1 antibody light (kappa) chain (CK) lacking the light chain variable region. The nucleotide sequence encoding the AH3/5D-IgG1 heavy chain is provided in SEQ ID NO:45; the amino acid sequence is provided in SEQ ID NO:44. The nucleotide sequence encoding the AA6/E3-IgG1 kappa chain is provided in SEQ ID NO:47; the amino acid sequence is provided in SEQ ID NO:46.

The bi-specific (AH3/5D-IgG1 and E3/AA6-IgG1) and tetra-specific (ABAB-IgG1) IgG1 binding agents were expressed and purified from stable transfected HEK293 cell line culture supernatant using protein A beads under conditions permitting disulfide bond formation and bi-valent molecule production. SDS-PAGE shows more than 90% purity of the purified ABAB-IgG1 with total molecular weight (light and heavy chains together) around 218 KDa on non-reduced gel (data not shown). The molecular weight of heavy chain is 68 KDa and light chain is 41 KDa showed on reduced gel.

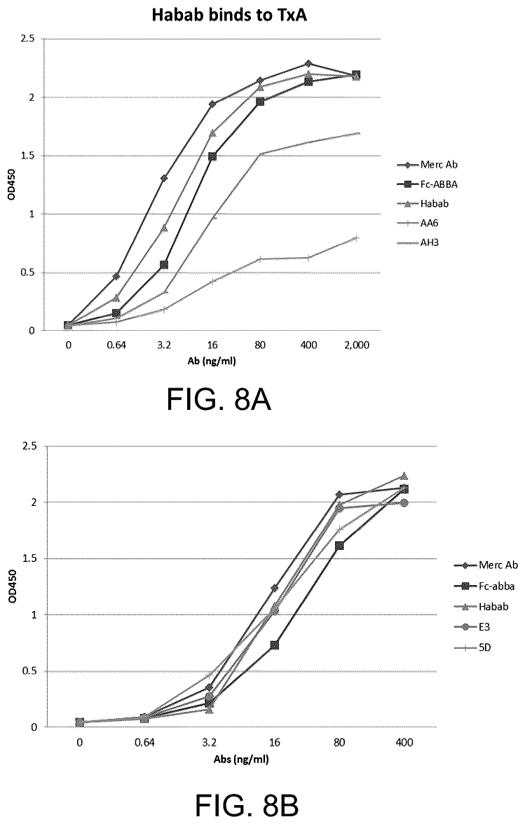

The binding of ABAB-IgG1 to TcdA and TcdB was determined. FIGS. 8A-8B illustrate the comparison of binding ABAB-IgG1 to both toxins with the individual components (AH3, AA6, E3, and 5D). FIG. 8A shows the results of experiments where plates were coated with 1 ug/ml TcdA (TxA). Serially diluted ABAB-IgG was added in concentrations of 0, 0.64, 3.2, 16, 80, 400 and 2,000 ng/ml. The plates were washed and Merck Ab (anti-TcdA), Fc-ABBA (ABAB-Fc), Habab (ABAB-IgG), and V_{H}H anti-TcdB monomers AA6 and AH3 were added in the indicated amounts (ng/ml). Appropriate labeled antibodies were used for detection. FIG. 8B shows the results of experiments where plates were coated with 1 ug/ml TcdB (TxB). Serially diluted ABAB-IgG was added in concentrations of 0, 0.64, 3.2, 16, 80 and 400 ng/ml. The plates were washed and Merck Ab (Anti-TcdB), Fc-abba (ABAB-Fc), Habab (ABAB-IgG), and V_{H}H anti-TcdB monomers E3 and 5D were added in the indicated amounts (ng/ml). Appropriate labeled antibodies were used for detection.

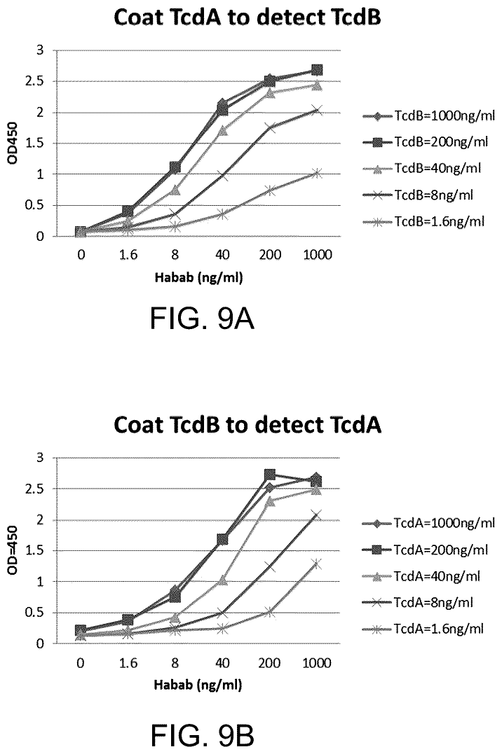

As expected, the tetra-specific antibody can bind to TcdA and TcdB simultaneously as determined by sandwich ELISA (FIGS. 9A-9B). In a first set of experiments, plates were coated with 1 ug/ml TcdA (TxA). Serially diluted ABAB-IgG (Habab) was added in concentrations of 0, 1.6, 8, 40, 200 and 1000 ng/ml. The plates were washed and the following amounts of TcdB were added: 1.6, 8, 40, 200, and 1000 ng/ml. Mouse anti-TxB antibodies (500×) and goat anti-mouse-IgG-HRP (3000×) antibodies were used for detection. The results provided in FIG. 9A show that TxB is detected by coating TxA, suggesting IgG-ABAB binds to TxA/B simultaneously. In a second set of experiments, plates were coated with 1 ug/ml TcdB (TxB). Serially diluted ABAB-IgG (Habab) was added in concentrations of 0, 1.6, 8, 40, 200 and 1000 ng/ml. The plates were washed and the following amounts of TcdA were added: 1.6, 8, 40, 200, and 1000 ng/ml. Mouse anti-TxA antibodies (500×) and goat anti-mouse-IgG-HRP (3000×) antibodies were used for detection. The results provided in FIG. 9B show that TxA is detected by coating TxB, again suggesting IgG-ABAB binds to TxA/B simultaneously.

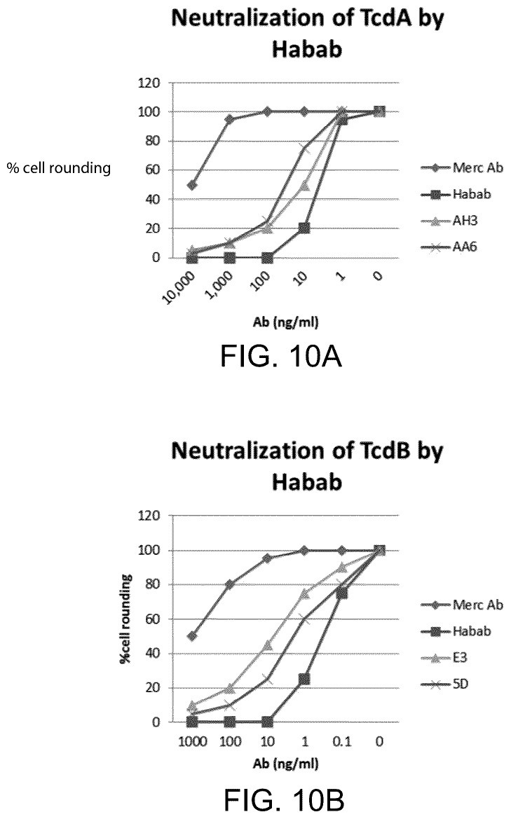

The neutralizing activities of ABAB-IgG1 against cytopathic effects of the toxins on cultured cells were also examined. TcdA (100 ng/ml, FIG. 10A) was mixed with serially diluted Merck anti-TcdA human monoclonal antibody, ABAB-IgG1 (Hababa), and V_{H}H anti-TcdA monomers AA6 and AH3 before adding to Vero cell monolayers in 100 ul culture medium and incubated at 37° C. for 24 hours. The results provided in FIG. 10A show that ABAB-IgG1 is at least 1000-fold more potent than Merck antibodies in neutralizing TcdA. In similar experiments, TcdB (10 pg/ml, FIG. 10B) was mixed with serially diluted Merck anti-TcdB human monoclonal antibody, ABAB-IgG1 (Hababa), and V_{H}H anti-TcdB monomers E3 and 5D before adding to Vero cell monolayers in 100 ul culture medium and incubated at 37° C. for 24 hours. The results provided in FIG. 10B show that ABAB-IgG1 is at least 1000-fold more potent than Merck antibodies in neutralizing TcdB.

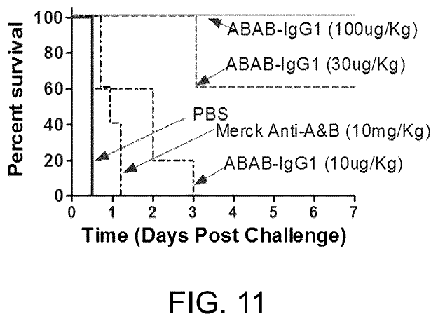

The in vivo neutralizing activities of ABAB-IgG1 were studied in a mouse model of CDI, the results of which are shown in FIG. 11. Mice were challenged with lethal dose of a mixed TcdA and TcdB (25 ng each toxin per mouse) and 4 hour later, ABAB-IgG (10, 30 or 100 μg/kg), a mixture of Merck anti-toxin A and anti-toxin B antibodies (10 mg/kg) or PBS was administered to the mice. The results demonstrate that the neutralizing activity of ABAB-IgG was much greater than the Merck antibody, and at lower concentrations.

### Animal Testing of ABAB-IgG

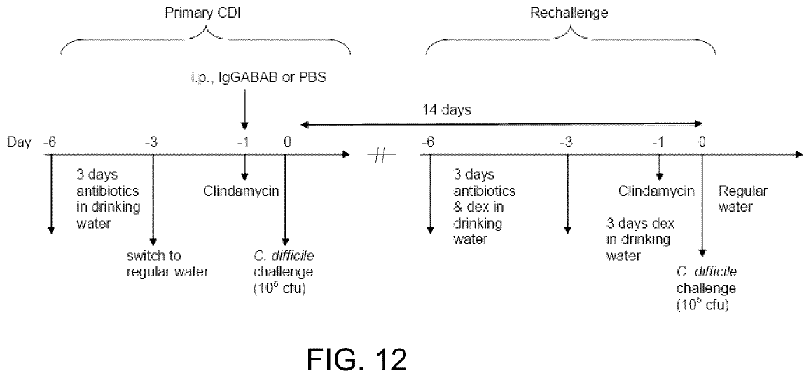

The ABAB-IgG binding agent was tested in both prophylactic treatment and re-challenge survival assays. FIG. 12 provides the experimental design of both studies. 6-8 week old female C57 mice were used, and the conditions included PBS: 10 ml/kg, i.p., n=14; ABAB-IgG: 200 ug/kg, i.p., n=10; ABAB-IgG: 1 mg/kg, i.p., n=10; ABAB-IgG: 5 mg/kg, i.p., n=10.

Table 10 provides a summary of the results seen with prophylactic treatment of mice against C. difficile spores (UK1, a 027/BI/NAP1 epidemic strain). ABAB-IgG or PBS was administered one day prior to administrating of C. difficile spores. As can be seen, ABAB-IgG showed dose-related prophylactic protection against CDI, where 5 mg/kg showed complete protection on all the parameters examined and 200 ug/kg was found to be more potent than 200 ug/kg of bi-specific V_{H}H fusion antibody ABA [41].

TABLE 10

| | Diarrhea | | | | | | | |
|---|---|---|---|---|---|---|---|---|
| | Occur- | Day 1 | Day 2 | Weight Change | | | | Sur- |
| | rence | score | score | Overall | Day 2 | Day 3 | Day 4 | vival |
| 200 | | — | — | — | √ | — | √ | √ |
| μg/kg | | | | | | | | |
| 1 mg/kg | √ | — | √ | — | √ | √ | — | √ |
| 5 mg/kg | √ | √ | √ | √ | √ | √ | √ | √ |

Table 11 provides a summary of the results seen with re-challenge of mice against C. difficile spores. ABAB-IgG or PBS was administered 15 days prior to administrating of C. difficile spores. As can be seen, one dose of ABAB-IgG showed some protection against the CDI caused by re-challenge of spores, but the protection was much less efficient compared to that during the primary challenge. This may be due to the drop of the antibody level with time and the generation of antibody in the PBS group following primary challenge.

TABLE 11

| | Diarrhea | | | | | | | |
|---|---|---|---|---|---|---|---|---|
| | Occur- | Day 1 | Day 2 | Weight Change | | | | Sur- |
| | rence | score | score | Overall | Day 2 | Day 3 | Day 4 | vival |
| 200 | √ | √ | — | — | — | — | — | — |
| μg/kg | | | | | | | | |
| 1 mg/kg | √ | — | — | — | — | — | √ | — |
| 5 mg/kg | — | — | — | √ | — | — | — | — |

Intestinal delivery of IgG-ABAB was also tested for protection of mice from fulminant CDI. After a single IgG-ABAB injection into mouse ceca after a laparotomy, mice were completely protected against fulminant CDI of death outcomes whereas 50% of control mice succumbed (data not shown). Disease progress and severity were assessed daily using a clinical scoring system modified from a previous publication [62], which included four criteria (activity level, posture, coat, and diarrhea) each graded on a scale from 0 to 4 and added together to generate a score with a maximum value of 16. A normal mouse would score 0 and a mouse found dead was scored as 16. Mice with scores equal to or higher than 11 should be euthanized. Only one mouse in the IgG-ABAB treatment group developed transient diarrhea whereas mice injected with PBS developed severe CDI disease symptoms (data not shown). Thus, Ig-ABAB manually delivered by injection into mouse intestines showed potent therapeutic efficacy.

### Expression, Purification and Evaluation of Binding Agents

A variety of selection criteria is used to select the binding agents generated in the experiments described in the approaches herein. First, each of the constructs defined herein can be used in transient transfections of 293T cells for making small-scale recombinant proteins by Protein A affinity chromatography. The production yield of each construct can be determined by quantitative ELISA. Second, binding activity of recombinant proteins can be screened using ELISA and surface plasmon resonance (SPR) to select constructs that preserve their original binding activities against the toxins. Third, the proteins are evaluated for neutralizing activity in in vitro assays (FIG. 3).

Accumulating observations indicate that polyreactivity and/or autoreactivity of in vivo recombinant binding agents are potential issues related to their in vivo safety and half-life. The application of the selected ABAB binding agents as a systemic binding agent for preventing primary acute CDI likely requires that the chimeric and humanized ABAB proteins are limited in polyreactivity and/or autoreactivity. Progress in protein proteomics has made it possible to screen for polyreactivity and autoreactivity of recombinant antibodies in vitro, which is a great tool for surrogate therapeutic antibodies. Therefore, selected humanized binding agents with good yield, high binding affinity, and potent neutralizing activity can be further tested for potential polyreactivity and autoreactivity using the auto-antigen microarray test and ProtoArray protein microarrays (Invitrogen).

From the above in vitro assays, candidate ABAB-Fc and ABAB-IgG binding agents can be evaluated for their in vivo toxicity, serum half-life, and immunogenicity.

Generation of S. cerevisiae Secreting ABAB (Sc-ABAB)

Means for in vivo production and delivery of the binding agents to the gut of subjects having CDI or at risk of developing CDI were developed. Because S. cerevisiae is genetically similar to S. boulardii [52,53] and genetic tools are readily available for S. cerevisiae, S. cerevisiae was first used for ABAB secretion validation.

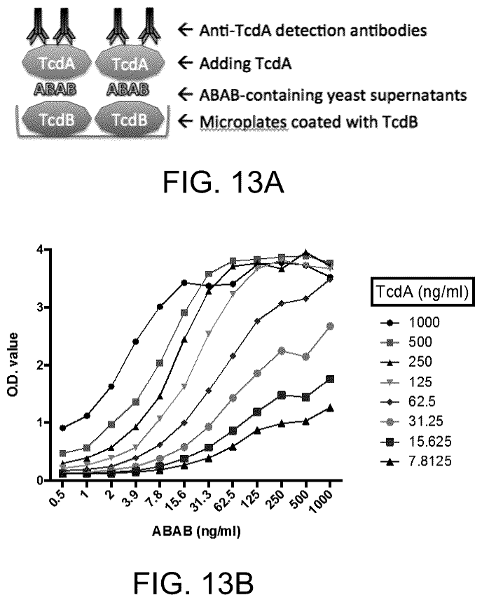

A novel bi-specific sandwich ELISA method was first developed to evaluate ABAB secretion. The setting utilizes purified TcdA and TcdB as binding antigens for ABAB bispecificity and α-TcdA antibodies for detection (FIG. 13A). For standardization, plates were coated with TcdB (1 ug/ml) into which was added serially diluted ABBA ((AH3-E3-E3-AA6)) standard. Serial diluted rTcdA (1 ug/ml to 7.8 ng/ml) was then added. The capture of TcdA was then measured by adding monoclonal antibody against TcdA followed by HRP conjugated secondary antibody. The results for the standard curves are shown in FIG. 13B. Based on these results, a standard curve derived using 125 ng/ml of rTcdA was chosen for determining secretion levels of ABAB in yeast culture supernatants and used for all subsequent ELISA.

A shuttle plasmid (pD1214-FAKS) containing origins of replication from both E. coli (pUC) and yeast (2 micron circle), as well as a yeast auxotrophic selection marker URA3 (conferring the ability to synthesize uracil), was obtained from DNA 2.0 (Newark, CA). The sequence encoding ABAB (SEQ ID NO:20), and His tag (SEQ ID NO:66) and D7 tag (SEQ ID NO: 112) at the N-terminus and C-terminus of ABAB respectively, was inserted into this plasmid backbone in which transcription was controlled by the strong constitutive yeast translational elongation factor promoter (PTEF) and extracellular secretion provided by fusion to the alpha mating factor secretion signal leader sequence (FAKS). The sequence of the resulting plasmid (pD1214-FAKS-His-hABAB-D7) is provided in SEQ ID NO:68.

Plasmid pD1214-FAKS-His-hABAB-D7 was transformed into the S. cerevisiae strain BY4741 (MATa his3A1 leu2A0 Met1540 ura340), an URA3 knockout S288C-derivative laboratory strain. Yeast transformants were then cultured in YNB medium containing dropout mix without uracil (6.8 g YNB, 20 g glucose, 2 g dropout mix in 1L of sterile ddH₂O) at 250 rpm at 30° C. overnight to reach O.D. 1 in a shaker. The cells were then centrifuged down and lysed by sonication in 1X SDS loading buffer. After sonication, total cell lysates were treated at 98° C. for 5 minutes before loading on a SDS gel. Same amount of yeast control cell lysates were loaded in each well except the control cells were not viable in YNB medium without uracil and therefore were cultured in YNB complimented with uracil.

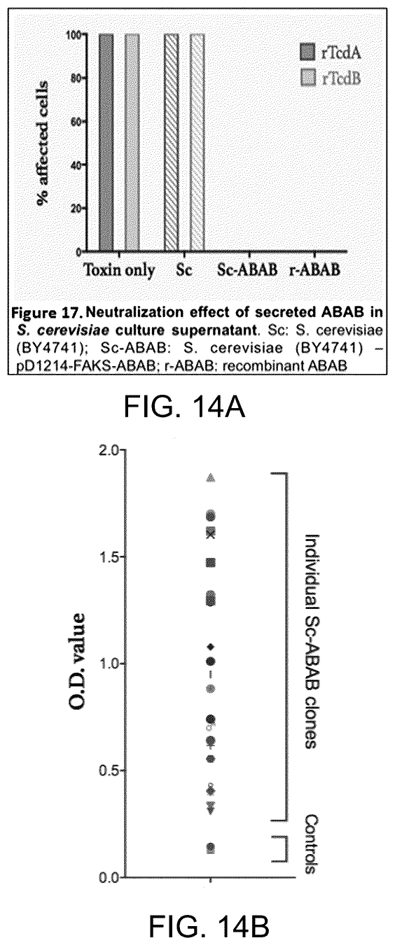

Culture supernatants from 25 yeast transformants as well as 3 yeast control colonies were centrifuged to spin down cells, and the cell-free supernatants were then diluted with 2.5% milk in PBS containing 0.05% of tween 20 at 1:3 ratio and screened by the ELISA as described above after 24 hrs of incubation in a shaker at 250 rpm and 30° C. FIG. 14B shows that all the yeast transformants secreted ABAB in culture supernatant compared to the culture supernatant from the yeast control colonies.

A cell-based neutralizing assay was used to assess the biological activity of secreted ABAB in culture supernatant. In this assay, sufficient amount of toxin A or toxin B to cause 100% cell rounding in 4 hours were added with PBS, cell-free culture supernatant from BY4741 control colony or BY4741-ABAB colony. Recombinant ABAB was used a positive control. The biological activity of secreted ABAB in culture supernatant was determined by the level of neutralizing activity to prevent cell rounding. Full length ABAB secreted from S. cerevisiae indeed retains its neutralizing activity when compared with purified recombinant ABAB (FIG. 14A). These combined results imply the plausibility of ABAB secretion by S. boulardii.

In further experiments, it was demonstrated that oral gavage of mice with Sc-ABAB at doses of 10¹⁰ CFU had no adverse effects on mice, and mice shed live Sc-ABAB as determined by plating feces on Sabouraud CAF-Agar (data not shown). Isolates recovered from mice retained their ability to produce functional ABAB using the assay described above.

### ABAB Secretion Optimization

ABAB secretion level is imperatively linked to in vivo therapeutic efficacy. Therefore, the possibility of further optimizing ABAB secretion by replacing the existing FAKS secretion signal with a number of commercially available secretion signals was explored. Secretion sequences facilitate co-translational or post-translational translocation of heterogeneous proteins into the endoplasmic reticulum and Golgi compartments prior to extracellular export. Although α-mating factor is a commonly used signal sequence for heterologous protein secretion that typically generates good yields of the secreted proteins in S. cerevisiae [69,70], studies have shown that other secretion sequences from other proteins such as inulinase or invertase could be more suitable for secreting certain heterologous proteins [71,72].

11 different commercially available secretion signals (Table 4; DNA 2.0, Newark, CA) were genetically fused with ABAB individually under the control of TEF promoter in the same pD1214 plasmid backbone. Plasmids encoding ABAB with alternative secretion signals include the following plasmids where the FAKS secretion signal is replaced by the noted new secretion signals from Table 4 and where both the his-tag and D7-tag are removed:

(SEQ ID NO: 70)
Plasmid pD1214-AKS-hABAB
(SEQ ID NO: 71)
Plasmid pD1214-AK-hABAB
(SEQ ID NO: 72)
Plasmid pD1214-AT-hABAB
(SEQ ID NO: 73)
Plasmid pD1214-AA-hABAB
(SEQ ID NO: 74)
Plasmid pD1214-GA-hABAB
(SEQ ID NO: 75)
Plasmid pD1214-IN-hABAB
(SEQ ID NO: 76)
Plasmid pD1214-IVS-hABAB
(SEQ ID NO: 77)
Plasmid pD1214-KP-hABAB
(SEQ ID NO: 78)
Plasmid pD1214-LZ-hABAB
(SEQ ID NO: 79)
Plasmid pD1214-SA-hABAB

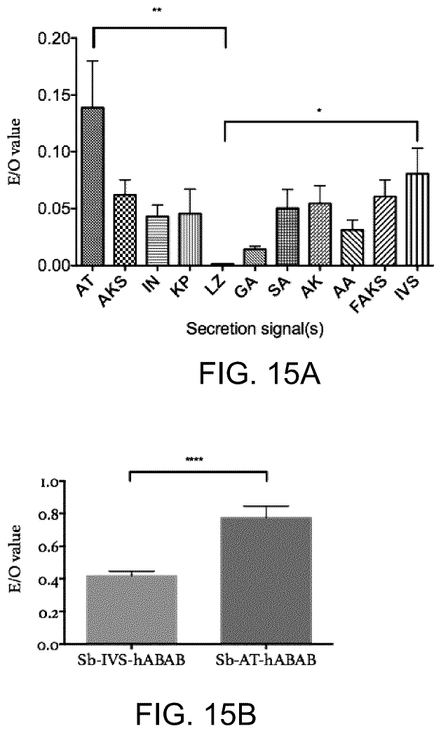

In addition, both the his-tag and D7-tag in the original ABAB construct (pD1214-FAKS-His-hABAB-D7) were removed to produce plasmid pD1214-FAKS-hABAB (SEQ ID NO:69) and culture incubation temperature was raised to 37° C. to better accommodate in vivo and clinical testing relevant scenarios. All 11 plasmids were then transformed in BY4741 and 5 independent colonies from each selective plate were selected to generate culture supernatants. The amount of secreted ABAB was determined by the same ELISA as described above. In addition, E/O value was used to provide a fair comparison across all groups. E/O value is defined by ELISA O.D. value normalizes against culture O.D. value. Two of the best secretion signals for ABAB were found to be AT and IVS (Table 4; FIG. 15A).

Due to the unavailability of an auxotrophic mutant strain for S. boulardii, another 2 μm-based plasmid carrying the aphAl gene encoding resistance to G418 (pCEV-G4-Km; SEQ ID NO: 80; a gift from Lars Nielsen & Claudia Vickers (Addgene plasmid #46819)) was used instead of pD1214 plasmids to confirm ABAB secretion in S. boulardii. The best two secretion signals for S. cerevisiae (AT and IVS) were fused with ABAB genetically and inserted in the pCEV-G4-Km plasmid backbone to generate plasmids pCEV-G4-Km-TEF-AT-hABAB* (SEQ ID NO: 81) and pCEV-G4-Km-TEF-IVS-hABAB* (SEQ ID NO:82). Both plasmids were used to transform S. boulardii (strain MYA796) and ABAB secretion with AT and IVS in S. boulardii was comparable with S. cerevisiae as determined by ELISA (FIG. 15B). A further construct, pCEV-G4-Km-TEF-AT-hABAB (SEQ ID NO:83), was prepared which differs from pCEV-G4-Km-TEF-AT-hABAB* in that it contains a molecular cloning site between the AT and hABAB sequence.

ABAB secretion was then further optimized by yeast codon optimization (yABAB) at the nucleotide level in the construct having the AT secretion signal, producing plasmid pCEV-G4-Km-TEF-AT-yABAB (SEQ ID NO:84). A sequence containing 40 nucleotides between PTEF and ABAB coding sequence was also found to be dispensable for ABAB secretion and removed resulting in plasmid pCEV-G4-Km-TEF-X40-AT-yABAB (SEQ ID NO:85). A further sequence containing two restriction cloning sites between AT and ABAB sequence was found to negatively impact ABAB secretion and therefore this sequence was also omitted (plasmid pCEV-G4-Km-TEF-AT-^{RS}yABAB; SEQ ID NO:115) for subsequent study to maximize ABAB secretion.

Next, the amount of secretion of the individual monomers was measured and AA6 was found to be secreted the least. To improve AA6 secretion, and thus further optimize ABAB secretion, a panel of key amino acid residues was utilized. A T83N mutation was found to improve AA6 secretion. In addition, S. boulardii carrying the hAA6 sequence was found to secrete more AA6 than the one carrying the yeast optimized yAA6 sequence. Therefore, a comparison was undertaken between ABAB carrying the T83N mutation within AA6 (AT-yABAB T83N; plasmid pCEV-G4-Km-TEF-AT-yABAB AA6T83N; SEQ ID NO:116) and ABAB where the yAA6 sequence was replaced by the hAA6 T83N sequence (AT-yABAB hAA6 T83N; plasmid pCEV-G4-Km-TEF-AT-yABAB hAA6T83N, which has the sequence of SEQ ID NO:90 but lacks the coding sequence for c-Myc)) to determined which sequence exhibited better secretion. It was found that there was no significance difference between these constructs and AT-yABAB hAA6 T83N was concluded as the final sequence moving forward. The nucleotide sequence encoding AT-yABAB hAA6 T83N is provided in plasmid pCEV-G4-Km-TEF-AT-yABAB hAA6T83N-tagless (SEQ ID NO:90). The amino acid sequence of AT-yABAB hAA6 T83N is provided in SEQ ID NO:117.

Generation of an Auxotrophic S. boulardii Strain

The expression plasmid encoding ABAB can be cloned into the S. boulardii strain. The S. boulardii strain can tolerate normal body temperature and acidic conditions better than S. cerevisiae, which can improve efficacy as a novel oral yeast-based therapeutic strategy. Two modifications to a wild-type S. boulardii strain can be made to preserve the in vivo stability of the expression plasmid conferred by the yeast URA3 metabolic selection marker: 1) a diploid auxotrophic mutant carrying a deletion in both chromosomal alleles of URA3 can be constructed, and 2) the endogenous 2 micron circle can be cured from S. boulardii to prevent unintended recombination from interfering with ABAB expression.

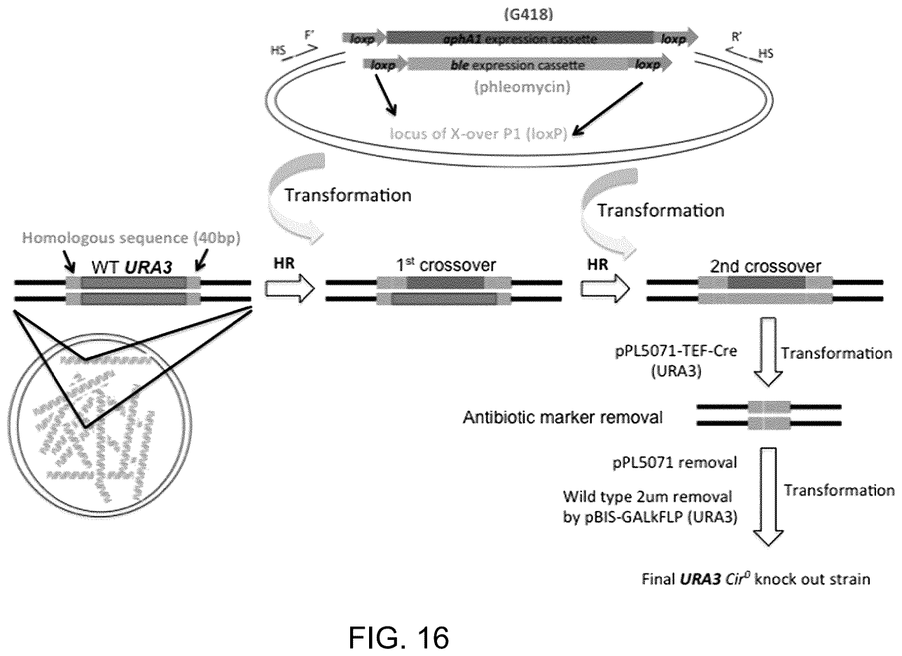

The most straightforward and efficient method for constructing auxotrophic mutants in wild-type Saccharomyces strains involves targeted deletion of chromosomally encoded genes by homologous recombination, which occurs at very high frequencies in Saccharomyces. Complete deletion of the targeted gene is preferred over selection of spontaneous mutations which can revert back to the wild type. Thus a gene deletion is preferred for the haploid state in S. cerevisiae which is typically induced from wild-type diploid via sporulation using a nutritionally poor growth medium and incubating at low temperature (30° C.). However, S. boulardii is sporulation deficient and recalcitrant to formation of haploid cells under normal sporulation conditions [64,65]. A two-step process for deletion of both chromosomal gene alleles (e.g. URA3) was used in which each deletion step can be selected for. The process is outlined schematically in FIG. 16.

All chromosomal deletions were carried out by lithium acetate-facilitated genetic transformation of linear DNA deletion cassettes. Lithium acetate-based transformation originated from a S. cerevisiae protocol and was found to be compatible with S. boulardii although S. boulardii was found to be much harder to transform [55,56]. The difference is around 100 fold. Transformation efficiency in S. cerevisiae can be improved by adjusting glucose concentration and heat shock time [74]. Therefore various glucose concentrations and heat shock times were incorporated in S. boulardii transformation for optimization. The best condition tested for S. boulardii was 2% glucose in preculture and 20 minutes of heat shock time at 42° C. and these conditions were used for all transformation procedures in all studies.

Two deletion cassettes containing the genes aphAl and ble, which confer resistance to G418 and phleomycin in yeast respectively, were generated by PCR using pCEV-G4-Km (SEQ ID NO:80) and pCEV-G4-Ph (SEQ ID NO:86) (a gift from Lars Nielsen & Claudia Vickers (Addgene plasmid #46820)) as templates. Both deletion cassettes are flanked by two locus of X-over P1 (loxP) in the same direction, allowing for antibiotic resistance genes spin out using Cre-recombinase. 40 base pairs of homologous sequences upstream of URA3 promoter (P_{URA3}) and downstream of the stop codon of URA3 were incorporated in PCR primers to generate two final deletion cassettes for site-specific gene deletion in S. boulardii (see FIG. 16). The exact sequence and location of URA3 gene on chromosome V on S. boulardii was mapped using URA3 gene annotation from online-published sequence from Saccharomyces genome database (SGD). Selection for crossover 1 replacing the first URA3 allele with aphAl deletion cassette is selected for using resistance to G418 [66]; the second crossover replacing the second URA3 allele with ble deletion cassette is selected for using resistance to phleomycin (FIG. 16). The replacement of both URA3 alleles with aphAl and ble deletion cassettes was evidenced by resistance to both antibiotics (data not shown) as well as lack of growth on minimal synthetic medium plates lacking uracil (data not shown). Yeast phenotype was also confirmed by growth on Sabouraud plate with chloramphenicol (100 ug/ml) (data not shown). In addition, three sets of unique primers targeting the URA3, aphAl or ble genes in the URA3 chromosomal region was designed and performed PCR using wild type (WT), URA3Δ::aphA1/URA3 (1^{st} crossover) and URA3Δ::aphA1/A::ble (2nd crossover) genomic DNA as templates. Expected PCR product sizes targeting the URA3, aphAl or ble genes in the URA3 chromosomal region are 766 bp, 1183 bp, and 662 bp respectively. DNA electrophoresis of PCR products from WT, 1^{st} crossover and 2^{nd} crossover clones using these three sets of unique primers confirmed the absence of URA3 alleles and integration of the aphAl and ble deletion cassettes of the 2^{nd} crossover strain.

The 2^{nd} crossover strain was then transformed with pPL5071_TEF1-Cre_URA3 (pPL5071; SEQ ID NO:95) to remove the aphAl and ble deletion cassettes. Strain carries pPL5071 expresses Cre recombinase constitutively under PTEF. Cre recombinase then targets loxp sequences flanking the aphAl and ble deletion cassettes; this causes the excision of the aphAl and ble deletion cassettes, leaving only one loxp site in the URA3 chromosomal region. Strains that underwent successful excision of the aphAl and ble deletion cassettes cannot grow in the presence of either G418 or phleomycin; yet retain the loss of both URA3 alleles, therefore can only grow on minimal synthetic medium plate in the presence of uracil and showed no growth on minimal synthetic medium plate without uracil supplement.

Removal of pPL5071 was achieved by growth in YPD and selecting for colonies later grown on minimal synthetic medium containing uracil and the pyrimidine analog 5-fluoro-orotic acid (5-FOA) [77]. Strains possessing pPL5071 carry the URA3 gene that can synthesize the toxic intermediate 5-fluorodeoxyuridine a potent inhibitor of thymidylate synthetase, which interrupts DNA synthesis and leads to cell death and allows selection of strains that have lost pPL5071. The absence of pPL5071 also was confirmed by pPL5071 specific primers by PCR and DNA electrophoresis of the PCR product.

The 2 um plasmid is a very stable 6.1 kb plasmid that is ubiquitous in Saccharomyces strains. This plasmid confers no selective advantage to the yeast host organism, and it is remarkably stable due to the presence of an efficient REP1-REP2-STB plasmid partitioning system [68]. S. boulardii strains used also contain this plasmid as confirmed via PCR. To remove the 2 um plasmid, pBIS-GALkFLP-URA3 (SEQ ID NO:87) was used to cure 2 um plasmid, followed by removal with uracil and 5-FOA. Loss of the 2 um plasmid was confirmed by PCR using primers specific for the origin of replication.

The auxotrophic strain of S. boulardii that results from these manipulations is termed S. boulardii URA3 Δ/Δ.

Auxotrophic S. boulardii Strain for In Situ Delivery of ABAB

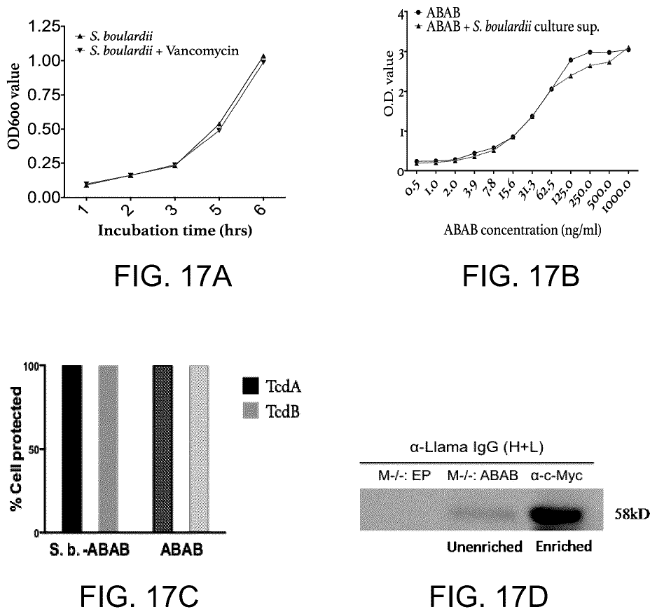

For constructing the auxotrophic S. boulardii strain for in situ delivery of ABAB, the aphAl cassette of the plasmid pCEV-G4-Km-TEF-X40-AT-yABAB (SEQ ID NO:85) was replaced by the URA3 cassette from pD plasmid to generate the plasmid pCEV-URA3-TEF-AT-yABAB-cMyc (SEQ ID NO:88). This plasmid was then used to transform S. boulardii URA3 Δ/Δ. The resulting strain secretes fully functional ABAB when compared with purified ABAB in a cell toxicity assay (FIG. 17C). Western blotting showed the corresponding ABAB band from S. boulardii culture supernatant using α-Llama antibodies conjugated with HRP (FIG. 17D). C-terminus end of ABAB contains c-Myc tag and can be further pulled down by a-c-Myc antibodies (FIG. 17D).

For empty plasmid (EP) control, AT-yABAB sequence was later removed from pCEV-URA3-TEF-AT-yABAB-cMyc (SEQ ID NO:88) to generate pCEV-URA3-TEF-cMyc (SEQ ID NO:89). S. boulardii URA3 Δ/Δ strain transformed with this plasmid results a strain complemented with URA3 but does not secrete ABAB. S. boulardii URA3 Δ/Δ strain secreting ABAB also showed no growth inhibition when cultured in YPD containing vancomycin (1 mg/ml) (FIG. 17A). This suggests S. boulardii can be co-administered with vancomycin typically used to treat CDI patients and secretes ABAB to treat ongoing CDI. In addition, purified ABAB is stable in culture supernatant collected from S. boulardii at O.D. 10 over 2 hours period of time suggests secreted ABAB is likely to diffuse out from S. boulardii without being degraded.

Safety Assessment of S. boulardii Delivered Orally to Antibiotic-Treated Mice

Prior to evaluating whether S. boulardii URA3 Δ/Δ expressing ABAB can protect mice in CDI models [20,33,62,78], a safety assessment was performed to determine safe doses of S. boulardii in antibiotic-treated mice. In this safety assessment mice were first supplied with an antibiotic cocktail in their daily drinking water for three days and then switched to regular water. One day before oral delivery of S. boulardii, mice were injected with clindamycin intraperitoneally. This completes the antibiotic treatment for the mice and S. boulardii was then orally delivered to the mice for safety assessment, which includes monitoring of daily weight change and persistence of S. boulardii in their stool samples of these antibiotic-treated mice. Mice exhibited no signs of illness and steadily weight increase during 6 days of monitoring when 10¹⁰ cells of S. boulardii were delivered orally consistent with the idea of S. boulardii as a GRAS organism. For the subsequent CDI mouse studies, however, only 10⁹ cells of S. boulardii were given due to the ease of pellet resuspension and less variability of the dosing amount to the mice, which can occur with high viscosity present in resuspension. S. boulardii also shows limited colonization in these antibiotic-treated mice GI tracts; three days after the final gavage, no detectable S. boulardii were recovered from Sabouroud plate (data not shown).

Protection of S. boulardii Expressing ABAB Against Primary CDI in Mice

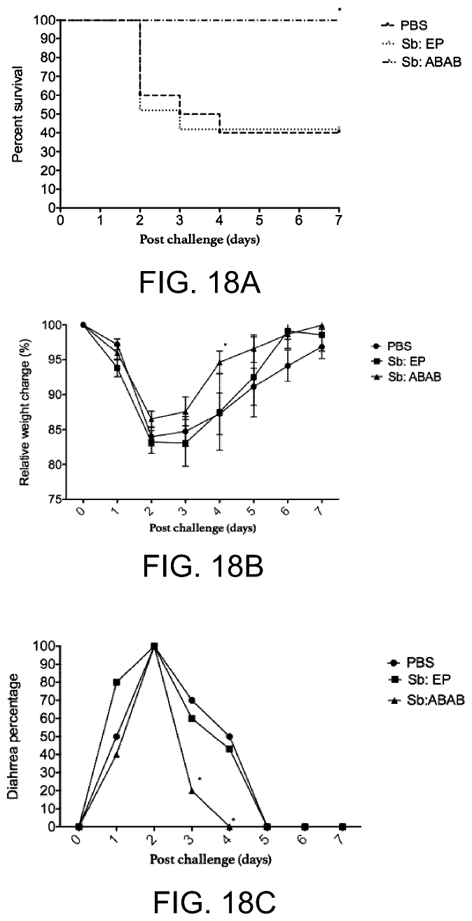

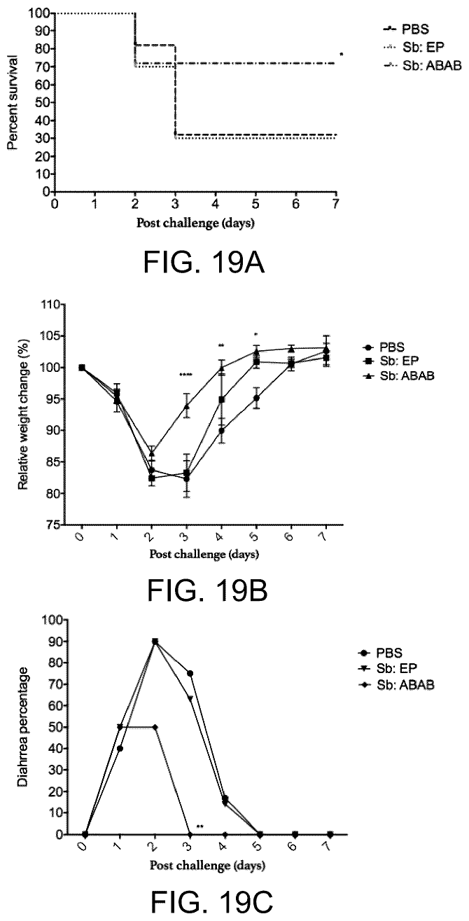

Protection of S. boulardii expressing ABAB was evaluated using established primary mouse CDI models. S. boulardii expressing ABAB was delivered either as preventative or treatment against primary CDI in mice. In brief, primary CDI was established in mice by supplementing a mixture of antibiotic into their drinking water for three days, and then intraperitoneal injection of clindamycin 24 hours prior to C. difficile spore challenge. 10⁵ C. difficile spores (UK1, a 027/BI/NAP1 epidemic strain) were gavaged in the mice to induce CDI. For preventative evaluation, mice started receiving an oral dose of S. boulardii the day after switching to regular drinking water, which continued every day for 7 days. For therapeutic evaluation, mice received an oral dose of S. boulardii at 6, 24, 48, and 72 hours after spore challenge. Controls included PBS and S. boulardii transformed with an empty plasmid. In both methods, mice receiving S. boulardii expressing ABAB were significantly protected against CDI-induced deaths (FIGS. 18A and 19A; PBS: negative control; Sb: EP: S. boulardii transformed with an empty plasmid; Sb: BAB: S. boulardii secreting ABAB). CDI mice typically suffered weight loss with most weight drops around day 2 to day 3 due to diarrhea and gradually recovered. Weights of mice receiving S. boulardii expressing ABAB recovered significantly sooner (FIGS. 18B and 19B) and had significant reduced percentage of diarrhea incidents after day 2 post challenge (FIGS. 18C and 19C).

Protection of S. boulardii Expressing ABAB Against Recurrence CDI in Mice

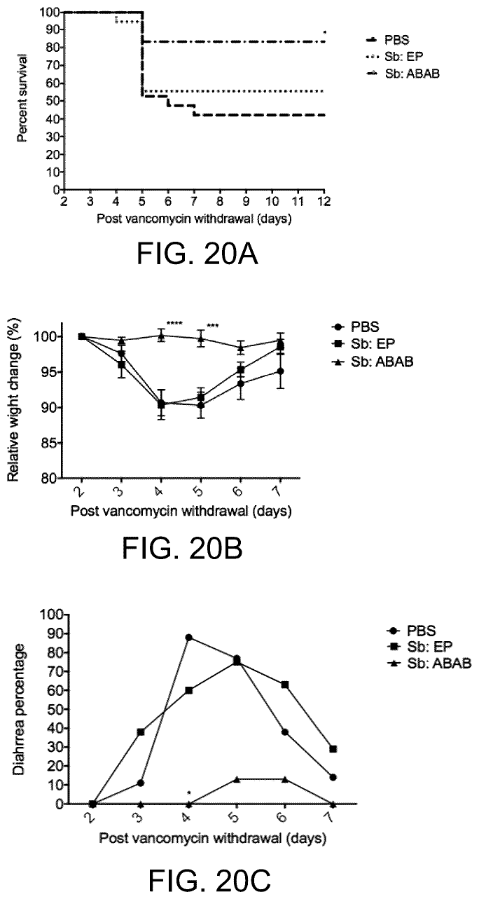

Protection of S. boulardii expressing ABAB was evaluated against recurrence CDI in mice. To induce recurrent CDI, mice were given three days of antibiotic cocktail in their daily drinking water. After three days of antibiotic water, mice were then switched back to drinking regular water. One day before oral delivery of 105 C. difficile spores (UK1, a 027/BI/NAP1 epidemic strain), mice were injected with clindamycin intraperitoneally. Six hours after spore challenge, regular water was changed to water containing 0.5 mg/ml of vancomycin for six days and regular water was switched back again for the rest of study. Mice typically develop signs of CDI after 4 days of vancomycin withdrawal without another C. difficile spore challenge. During the course of recurrence model, S. boulardii was orally delivered along with vancomycin water once every day for 12 days. This model is used to evaluate protection efficacy of S. boulardii expressing ABAB for preventing CDI recurrence in mice. Survival rate, weight loss and diarrhea incident of these mice were monitored on a daily basis. Controls included PBS and S. boulardii transformed with an empty plasmid. Mice receiving S. boulardii expressing ABAB were significantly protected against recurrence-induced CDI deaths (FIG. 20A; PBS: negative control; Sb: EP: S. boulardii transformed with an empty plasmid; Sb: BAB: S. boulardii secreting ABAB). Similar to primary CDI mice, recurrent CDI mice also typically suffered weight loss with most weight drops around day 4 to day 5 after vancomycin water withdrawal. Mice receiving S. boulardii expressing ABAB were significantly protected from weight loss (FIG. 20B) and had significant reduced percentage of diarrhea recurrence incidents (FIG. 20C).

### Stability Optimization of ABAB Cassette Through Chromosomal Integration

Genome editing using a CRISPR-Cas9 based system has been recently demonstrated both in S. cerevisiae and S. boulardii [79-81]. In addition, large fragment deletion can be achieved by targeting two guide sequences simultaneously [82]. Foreign genes are typically more steadily maintained when integrated into chromosomes versus introduced via plasmids when there is no selection pressure. However, chromosomal integration often requires multiple rounds of integration to achieve high copies. A protocol reported in a recent publication overcame this hurdle through targeting multiple copies of common sequences such as δ sites in S. cerevisiae genome through CRISPR-induced double stain breaks and achieved concurrent integration of large fragments in these sites [83]. DNA double strain break can be repaired either by non-homologous end joining or homologous recombination; however, when endogenous homologous sequences are present, host preferentially uses homologous sequences to repair DNA double strain break by homologous recombination [83].

δ sites are long terminal repeats (LTRs) belong to the Ty element I and II and are the most abundant LTRs in S. cerevisiae. There are five types of Ty elements (1-5) represented by the class II transposon (retrotransposon) that is more commonly found in S. cerevisiae. It is estimated that there are about 51 retrotransposons (Ty1-5) and 251 δ sites across S. cerevisiae genomes [84]. Such δ sites are appealing target sequences for ABAB expression cassette integration into S. boulardii chromosomes. However, much less is known about δ sites in S. boulardii. Therefore, Ty1-H3 (Genbank accession no. M18706) was first used as a probe to survey Ty1-2 elements in S. boulardii strain MYA796 (ATCC, Manassas, VA) (draft genome obtained from NCBI) to identify possible Ty1-2 elements and their δ sites in the S. boulardii genome. Surprisingly, no full Ty1-2 elements were found in MYA796. A total of 57 δsites were found; this includes 44 full δsites and 12 partial sites as well as a partial Ty element containing 1 full δ site identified across all 16 chromosomes (Table 12).

TABLE 12
Number of δ sites and their distribution on
MYA796 chromosomes

| | | | | Partial Ty1, 2 |
|---|---|---|---|---|
| | Full δ | Full Ty1, | Partial δ site | element with |
| | site | 2 elements | 60 < X < 200 bp | full δ site |
| Ch I | 0 | 0 | 1 | 0 |
| Ch II | 0 | 0 | 0 | 0 |
| Ch III | 1 | 0 | 0 | 0 |
| Ch IV | 5 | 0 | 1 | 1 |
| Ch V | 2 | 0 | 1 | 0 |
| Ch VI | 2 | 0 | 0 | 0 |
| Ch VII | 8 | 0 | 1 | 0 |
| Ch VIII | 2 | 0 | 0 | 0 |
| Ch IX | 3 | 0 | 0 | 0 |
| Ch X | 3 | 0 | 1 | 0 |
| Ch XI | 0 | 0 | 0 | 0 |
| Ch XII | 8 | 0 | 1 | 0 |
| Ch XIII | 2 | 0 | 1 | 0 |
| Ch XIV | 1 | 0 | 0 | 0 |
| Ch XV | 2 | 0 | 4 | 0 |
| Ch XVI | 5 | 0 | 1 | 0 |
| Total | 44 | 0 | 12 | 1 |

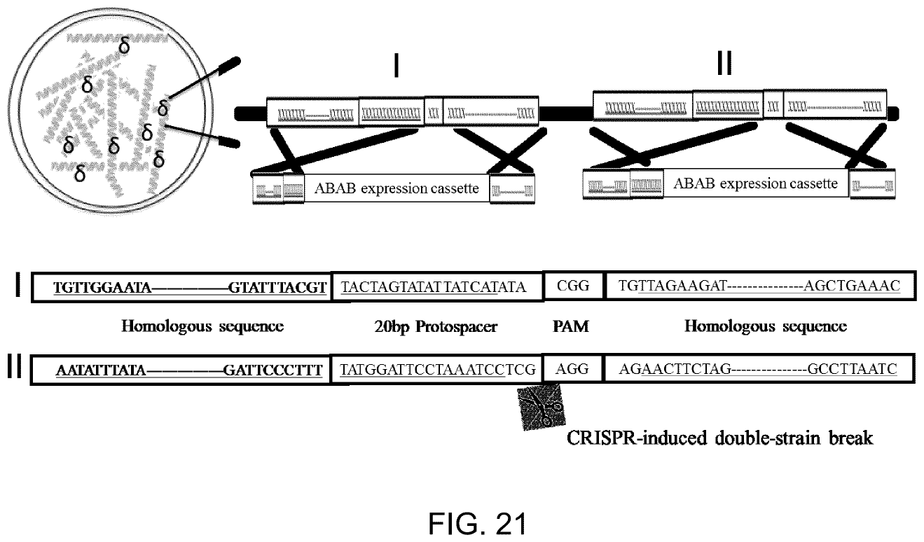

Due to S. boulardii diploid state; there are about 114 δsites across the S. boulardii genome. To allow simple multiple δ site targeting by CRISPR, all 57 δsite sequences were compiled for multiple sequence alignment using MUSCLE to identify protospacer adjacent motif (PAM) sites that present in high numbers among the 57 δsequences. Two PAM sites were chosen based on the highest number of δ sequences having uniformity in protospacers as the upstream and downstream sequences. The sequences of these PAM sites are illustrated in FIG. 21 and the specific sequences are as follows:

PAM Site I
(SEQ ID NO: 93)

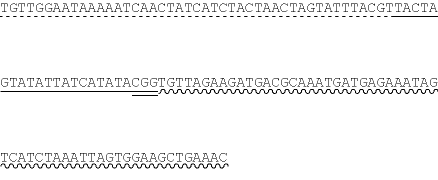

PAM Site II
(SEQ ID NO: 94)

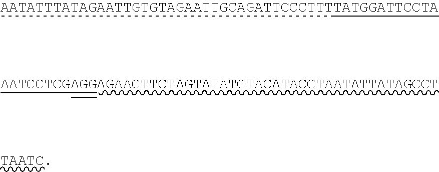

In both Pam Site I and Pam Site II, the sequences underscored by a dashed line correspond to the upstream homologous sequences; the sequences underscored by a single line correspond to the 20 bp protospacers; the sequences underscored by a double line correspond to the PAM sequences; the sequences underscored by a wavy line correspond to the downstream homologous sequences.

These two PAM sites, accompanied by their common upstream and downstream homologous sequences within the δsites, allow simple chromosomal integration of ABAB expression cassettes into S. boulardii genomes. ABAB integration cassettes containing homologous recombination sequences were generated by PCR using primers containing the upstream homologous sequences with the last three nucleotides removed at the 3′ end and the downstream homologous sequences with the first two nucleotides removed at the 5′ end and the corresponding annealing sequences needed for PCR using plasmid pCEV-G4-Km-TEF-AT-yABAB hAA6T83N-tagless as template (SEQ ID NO:90).

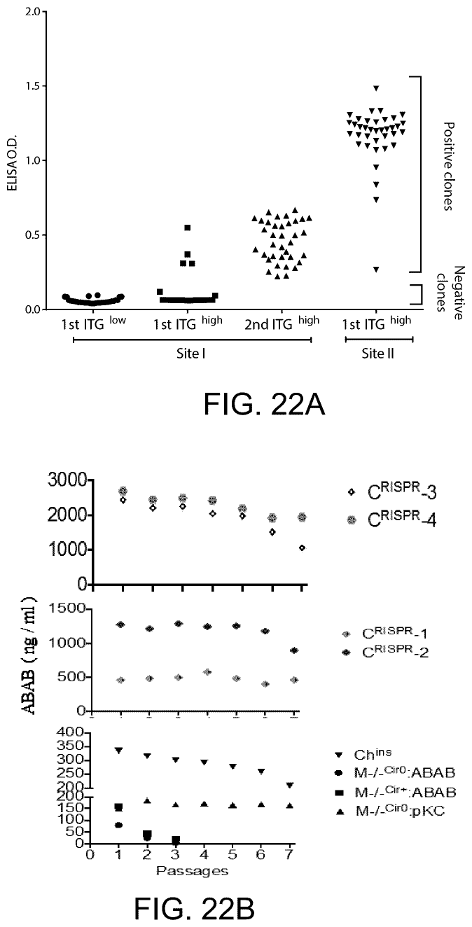

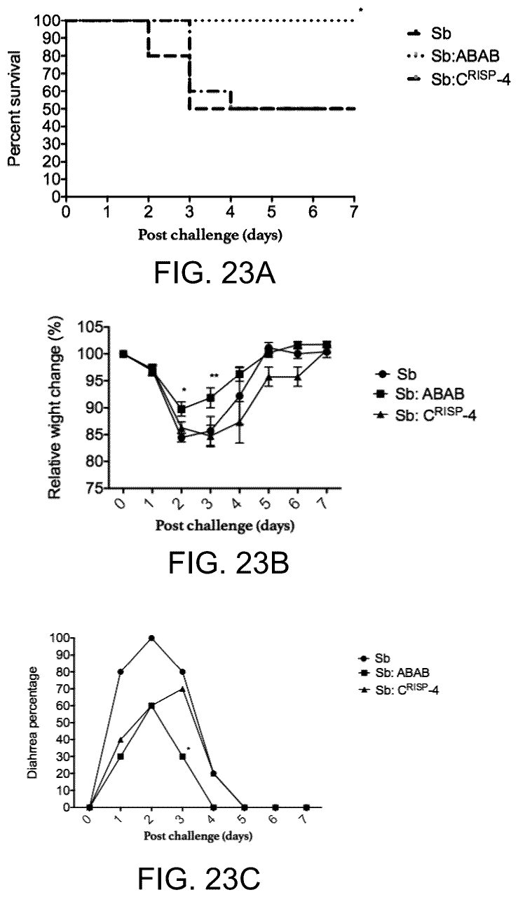

PCR products of the ABAB integration cassette with CRISPR plasmids contain the corresponding guide sequence (pCRI-Sb-81 (SEQ ID NO:91) and pCRI-Sb-82 (SEQ ID NO:92)) were then cotransformed with S. bouladii for ABAB integrations into chromosomes independently and sequentially to target PAM site I and PAM site II. The ratio of PCR product to CRISPR plasmid was found to be important for generating successful integration clones (FIG. 22A; ITG^{low} versus ITG^{high}). In addition, a repeat transformation of the highest ABAB secretion clone from ITG^{high} group with the same integration cassette and CRISPR plasmid did not further improve the overall ABAB secretion of independent clones (FIG. 22A; 2^{nd} ITG^{high}). ABAB secretion of the highest ABAB secretion clone (C^{RISPR}-2) from ITG^{high} group was then further improved by cotransforming the second set of ABAB integration cassette containing the homologous recombination sequences and its corresponding guide sequence in C^{RISPR} plasmid targeting site II (FIG. 22A). Two highest ABAB secretion clones, C^{RISP}-3 and C^{RISPR}-4 were selected. ABAB secretion amount and stability over time of these four representative clones are shown in FIG. 22B. A preliminary mouse CDI study was performed. However, C^{RISPR}-4 was found to be not better than previously M−/−: ABAB clone that showed protection in a number of mouse CDI models (FIG. 23).

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
1. Cloud, J. & Kelly, C. P. Update on Clostridium difficile associated disease. Curr Opin Gastroenterol 23, 4-9 (2007).
2. Kelly, C. P. & LaMont, J. T. Clostridium difficile—more difficult than ever. N Engl J Med 359, 1932-1940 (2008).
3. Barbut, F., et al. Epidemiology of recurrences or reinfections of Clostridium difficile-associated diarrhea. J Clin Microbiol 38, 2386-2388 (2000).
4. Hassoun, A. & Ibrahim, F. Use of intravenous immunoglobulin for the treatment of severe Clostridium difficile colitis. Am J Geriatr Pharmacother 5, 48-51 (2007).
5. Shahani, L. & Koirala, J. Intravenous immunoglobulin in treatment of Clostridium difficile colitis. BMJ Case Rep 2012 (2012).
6. Saito, T., et al. Evidence of intravenous immunoglobulin as a critical supportive therapy against Clostridium difficile toxin-mediated lethality in mice. J Antimicrob Chemother 66, 1096-1099 (2011).
7. Abougergi, M. S. & Kwon, J. H. Intravenous immunoglobulin for the treatment of Clostridium difficile infection: a review. Dig Dis Sci 56, 19-26 (2011).
8. Sokol, H., Maury, E., Seksik, P., Cosnes, J. & Beaugerie, L. Single immunoglobulin infusion can reverse hemodynamic failure associated with severe Clostridium difficile colitis. Am J Gastroenterol 104, 2649-2650 (2009).
9. Lowy, I., et al. Treatment with monoclonal antibodies against Clostridium difficile toxins. N Engl J Med 362, 197-205 (2010).
10. Louie, T. J., et al. Fidaxomicin versus vancomycin for Clostridium difficile infection. The New England journal of medicine 364, 422-431 (2011).
11. Rao, K. & Young, V. B. Fecal Microbiota Transplantation for the Management of Clostridium difficile Infection. Infectious disease clinics of North America 29, 109-122 (2015).
12. Vyas, D., Aekka, A. & Vyas, A. Fecal transplant policy and legislation. World journal of gastroenterology: WJG 21, 6-11 (2015).
13. Tonna, I. & Welsby, P. D. Pathogenesis and treatment of Clostridium difficile infection. Postgrad Med J 81, 367-369 (2005).
14. McFarland, L. V., Elmer, G. W. & Surawicz, C. M. Breaking the cycle: treatment strategies for 163 cases of recurrent Clostridium difficile disease. The American journal of gastroenterology 97, 1769-1775 (2002).
15. O'Neill, G. L., Beaman, M. H. & Riley, T. V. Relapse versus reinfection with Clostridium difficile. Epidemiol Infect 107, 627-635 (1991).
16. Wilcox, M. H., Fawley, W. N., Settle, C. D. & Davidson, A. Recurrence of symptoms in Clostridium difficile infection—relapse or reinfection? J Hosp Infect 38, 93-100 (1998).
17. Johnson, S., Adelmann, A., Clabots, C. R., Peterson, L. R. & Gerding, D. N. Recurrences of Clostridium difficile diarrhea not caused by the original infecting organism. J Infect Dis 159, 340-343 (1989).
18. Tang-Feldman, Y., Mayo, S., Silva Jr, J., Jr. & Cohen, S. H. Molecular analysis of Clostridium difficile strains isolated from 18 cases of recurrent Clostridium difficile-associated diarrhea. J Clin Microbiol 41, 3413-3414 (2003).
19. Johnson, S. Recurrent Clostridium difficile infection: causality and therapeutic approaches. Int J Antimicrob Agents 33 Suppl 1, S33-36 (2009).
20. Sun, X., et al. Mouse relapse model of Clostridium difficile infection. Infection and immunity 79, 2856-2864 (2011).
21. Seal, D., et al. Treatment of relapsing Clostridium difficile diarrhoea by administration of a non-toxigenic strain. Eur J Clin Microbiol 6, 51-53 (1987).
22. Kuehne, S. A., et al. The role of toxin A and toxin B in Clostridium difficile infection. Nature (2010).
23. Lyras, D., et al. Toxin B is essential for virulence of Clostridium difficile. Nature 458, 1176-1179 (2009).
24. Libby, J. M. & Wilkins, T. D. Production of antitoxins to two toxins of Clostridium difficile and immunological comparison of the toxins by cross-neutralization studies. Infection and immunity 35, 374-376 (1982).
25. Katchar, K., et al. Association between IgG2 and IgG3 subclass responses to toxin A and recurrent Clostridium difficile-associated disease. Clinical gastroenterology and hepatology: the official clinical practice journal of the American Gastroenterological Association 5, 707-713 (2007).
26. Ghose, C., et al. Transcutaneous immunization with Clostridium difficile toxoid A induces systemic and mucosal immune responses and toxin A-neutralizing antibodies in mice. Infection and immunity 75, 2826-2832 (2007).
27. Sougioultzis, S., et al. Clostridium difficile toxoid vaccine in recurrent C. difficile-associated diarrhea. Gastroenterology 128, 764-770 (2005).
28. Kyne, L., Warny, M., Qamar, A. & Kelly, C. P. Association between antibody response to toxin A and protection against recurrent Clostridium difficile diarrhoea. Lancet 357, 189-193 (2001).
29. Leav, B. A., et al. Serum anti-toxin B antibody correlates with protection from recurrent Clostridium difficile infection (CDI). Vaccine 28, 965-969 (2010).
30. Fernie, D. S., Thomson, R. O., Batty, I. & Walker, P. D. Active and passive immunization to protect against antibiotic associated caecitis in hamsters. Dev Biol Stand 53, 325-332 (1983).
31. Kim, P. H., Iaconis, J. P. & Rolfe, R. D. Immunization of adult hamsters against Clostridium difficile-associated ileocecitis and transfer of protection to infant hamsters. Infection and immunity 55, 2984-2992 (1987).
32. Lyerly, D. M., Bostwick, E. F., Binion, S. B. & Wilkins, T. D. Passive immunization of hamsters against disease caused by Clostridium difficile by use of bovine immunoglobulin G concentrate. Infection and immunity 59, 2215-2218 (1991).
33. Wang, H., et al. A chimeric toxin vaccine protects against primary and recurrent Clostridium difficile infection. Infection and immunity 80, 2678-2688 (2012).
34. Hamers-Casterman, C., et al. Naturally occurring antibodies devoid of light chains. Nature 363, 446-448 (1993).
35. van der Linden, R., et al. Induction of immune responses and molecular cloning of the heavy chain antibody repertoire of Lama glama. J Immunol Methods 240, 185-195 (2000).
36. Harmsen, M. M. & De Haard, H. J. Properties, production, and applications of camelid single-domain antibody fragments. Appl Microbiol Biotechnol 77, 13-22 (2007).
37. Arbabi Ghahroudi, M., Desmyter, A., Wyns, L., Hamers, R. & Muyldermans, S. Selection and identification of single domain antibody fragments from camel heavy-chain antibodies. FEBS Lett 414, 521-526 (1997).
38. van der Linden, R. H., et al. Comparison of physical chemical properties of llama V_{H}H antibody fragments and mouse monoclonal antibodies. Biochim Biophys Acta 1431, 37-46 (1999).
39. Dumoulin, M., et al. Single-domain antibody fragments with high conformational stability. Protein Sci 11, 500-515 (2002).
40. Hussack, G., Hirama, T., Ding, W., Mackenzie, R. & Tanha, J. Engineered single-domain antibodies with high protease resistance and thermal stability. PloS one 6, e28218 (2011).
41. Yang, Z., et al. A novel multivalent, single-domain antibody targeting TcdA and TcdB prevents fulminant Clostridium difficile infection in mice. The Journal of infectious diseases 210, 964-972 (2014).
42. McFarland, L. V. Systematic review and meta-analysis of Saccharomyces boulardii in adult patients. World J. Gastroenterol. 16, 2202-2222 (2010).
43. Elmer, G. W., McFarland, L. V., Surawicz, C. M., Danko, L. & Greenberg, R. N. Behaviour of Saccharomyces boulardii in recurrent Clostridium difficile disease patients. Aliment. Pharmacol. Ther. 13, 1663-1668 (1999).
44. Surawicz, C. M., et al. The search for a better treatment for recurrent Clostridium difficile disease: use of high-dose vancomycin combined with Saccharomyces boulardii. Clin. Infect. Dis. 31, 1012-1017 (2000).
45. McFarland, L. V., et al. A randomized placebo-controlled trial of Saccharomyces boulardii in combination with standard antibiotics for Clostridium difficile disease. Jama 271, 1913-1918 (1994).
46. Czerucka, D., Piche, T. & Rampal, P. Review article: yeast as probiotics—Saccharomyces boulardii. Alimentary pharmacology & therapeutics 26, 767-778 (2007).
47. Sougioultzis, S., et al. Saccharomyces boulardii produces a soluble anti-inflammatory factor that inhibits NF-kappaB-mediated IL-8 gene expression. Biochemical and biophysical research communications 343, 69-76 (2006).
48. Qamar, A., et al. Saccharomyces boulardii stimulates intestinal immunoglobulin A immune response to Clostridium difficile toxin A in mice. Infection and immunity 69, 2762-2765 (2001).
49. Chen, X., et al. Saccharomyces boulardii inhibits ERK1/2 mitogen-activated protein kinase activation both in vitro and in vivo and protects against Clostridium difficile toxin A-induced enteritis. The Journal of biological chemistry 281, 24449-24454 (2006).
50. Barc, M. C., et al. Molecular analysis of the digestive microbiota in a gnotobiotic mouse model during antibiotic treatment: Influence of Saccharomyces boulardii. Anaerobe 14, 229-233 (2008).
51. Gorlani, A., de Haard, H. & Verrips, T. Expression of V_{H}Hs in Saccharomyces cerevisiae. Methods in molecular biology 911, 277-286 (2012).
52. Khatri, I., et al. Gleaning evolutionary insights from the genome sequence of a probiotic yeast Saccharomyces boulardii. Gut Pathog. 5, 30 (2013).
53. Batista, T. M., Marques, E. T., Jr., Franco, G. R. & Douradinha, B. Draft Genome Sequence of the Probiotic Yeast Saccharomyces cerevisiae var. boulardii Strain ATCC MYA-796. Genome announcements 2, 578-579 (2014).
54. Hamedi, H., et al. Generation of a uracil auxotroph strain of the probiotic yeast Saccharomyces boulardii as a host for the recombinant protein production. Avicenna journal of medical biotechnology 5, 29-34 (2013).
55. Hudson, L. E., et al. Functional heterologous protein expression by genetically engineered probiotic yeast Saccharomyces boulardii. PLoS One 9, e112660 (2014).
56. Douradinha, B., et al. Novel insights in genetic transformation of the probiotic yeast Saccharomyces boulardii. Bioengineered 5, 21-29 (2014).
57. Fietto, J. L., et al. Molecular and physiological comparisons between Saccharomyces cerevisiae and Saccharomyces boulardii. Canadian journal of microbiology 50, 615-621 (2004).
58. Martins, F. S., et al. Screening of yeasts as probiotic based on capacities to colonize the gastrointestinal tract and to protect against enteropathogen challenge in mice. The Journal of general and applied microbiology 51, 83-92 (2005).
59. Martins, F. S., Veloso, L. C., Arantes, R. M. & Nicoli, J. R. Effects of yeast probiotic formulation on viability, revival and protection against infection with Salmonella enterica ssp. enterica serovar Typhimurium in mice. Lett. Appl. Microbiol. 49, 738-744 (2009).
60. Wu, D., Teng, D., Wang, X., Dai, C. & Wang, J. Saccharomyces boulardii prevention of the hepatic injury induced by Salmonella Enteritidis infection. Can. J. Microbiol. 60, 681-686 (2014).
61. Elmer, G. W. & Corthier, G. Modulation of Clostridium difficile induced mortality as a function of the dose and the viability of the Saccharomyces boulardii used as a preventative agent in gnotobiotic mice. Can. J. Microbiol. 37, 315-317 (1991).
62. Chen, X., et al. A mouse model of Clostridium difficile-associated disease. Gastroenterology 135, 1984-1992 (2008).
63. Pawlowski, S. W., et al. Murine model of Clostridium difficile infection with aged gnotobiotic C57BL/6 mice and a BI/NAP1 strain. The Journal of infectious diseases 202, 1708-1712 (2010).
64. McFarland, L. V. Saccharomyces boulardii is not Saccharomyces cerevisiae. Clin. Infect. Dis. 22, 200-201 (1996).
65. Edwards-Ingram, L., et al. Genotypic and physiological characterization of Saccharomyces boulardii, the probiotic strain of Saccharomyces cerevisiae. Appl. Environ. Microbiol. 73, 2458-2467 (2007).
66. Panchal, C. J., Whitney, G. K. & Stewart, G. G. Susceptibility of Saccharomyces spp. and Schwanniomyces spp. to the aminoglycoside antibiotic G418. Appl. Environ. Microbiol. 47, 1164-1166 (1984).
67. Tsalik, E. L. & Gartenberg, M. R. Curing Saccharomyces cerevisiae of the 2 micron plasmid by targeted DNA damage. Yeast 14, 847-852 (1998).
68. Chan, K. M., Liu, Y. T., Ma, C. H., Jayaram, M. & Sau, S. The 2 micron plasmid of Saccharomyces cerevisiae: a miniaturized selfish genome with optimized functional competence. Plasmid 70, 2-17 (2013).
69 Brake, A. J., et al. Alpha-factor-directed synthesis and secretion of mature foreign proteins in Saccharomyces cerevisiae. Proceedings of the National Academy of Sciences of the United States of America 81, 4642-4646 (1984).
70. Porro, D., Sauer, M., Branduardi, P. & Mattanovich, D. Recombinant protein production in yeasts. Molecular biotechnology 31, 245-259 (2005).
71. Chung, B. H., Nam, S. W., Kim, B. M. & Park, Y. H. Highly efficient secretion of heterologous proteins from Saccharomyces cerevisiae using inulinase signal peptides. Biotechnology and bioengineering 49, 473-479 (1996).
72. Hahm, M. S. & Chung, B. H. Secretory expression of human growth hormone in Saccharomyces cerevisiae using three different leader sequences. Biotechnology and Bioprocess Engineering 6, 306-309 (2001).
73. Gietz, R. D. & Schiestl, R. H. High-efficiency yeast transformation using the LiAc/SS carrier DNA/PEG method. Nat. Protoc. 2, 31-4 (2007).
74. Konishi, T. & Harata, M. Improvement of the transformation efficiency of Saccharomyces cerevisiae by altering carbon sources in pre-culture. Biosci. Biotechnol. Biochem. 78, 1090-1093 (2014).
75. Gatignol, A., Baron, M. & Tiraby, G. Phleomycin resistance encoded by the ble gene from transposon Tn 5 as a dominant selectable marker in Saccharomyces cerevisiae. MGG Mol. Gen. Genet. 207, 342-348 (1987).
76. Macdonald, C. & Piper, R. C. Puromycin- and methotrexate-resistance cassettes and optimized Cre-recombinase expression plasmids for use in yeast. Yeast 32, 423-438 (2015).
77. Boeke, J. D., LaCroute, F. & Fink, G. R. A positive selection for mutants lacking orotidine-5′-phosphate decarboxylase activity in yeast: 5-fluoro-orotic acid resistance. Mol Gen Genet 197, 345-346 (1984).
78. Yang, Z. et al. Intravenous adenovirus expressing a multi-specific, single-domain antibody neutralizing TcdA and TedB protects mice from Clostridium difficile infection. Pathog. Dis. 74, ftw078 (2016).
79. DiCarlo, J. E. et al. Genome engineering in Saccharomyces cerevisiae using CRISPR-Cas systems. Nucleic Acids Res. 41, 4336-43 (2013).
80. Bao, Z. et al. Homology-Integrated CRISPR-Cas (HI-CRISPR) System for One-Step Multigene Disruption in Saccharomyces cerevisiae. ACS Synth. Biol. 4, 585-594 (2015).
81. Liu, J.-J. et al. Metabolic engineering of a probiotic Saccharomyces boulardii. Appl. Environ. Microbiol. AEM.00057-16 (2016). doi: 10.1128/AEM.00057-16
82. Hao, H. et al. Large fragment deletion using a CRISPR/Cas9 system in Saccharomyces cerevisiae. (2016). doi: 10.1016/j.ab.2016.07.008.This
83. Shi, S., Liang, Y., Zhang, M. M., Ang, E. L. & Zhao, H. A highly efficient single-step, markerless strategy for multi-copy chromosomal integration of large biochemical pathways in Saccharomyces cerevisiae. Metab. Eng. 33, 19-27 (2016).
84. Kim, J. M. et al. Transposable Elements and Genome Organization: A Comprehensive Survey of Retrotransposons Revealed by the Complete Saccharomyces cerevisiae. Genome Research. 8 (5): 464-478 (1998).
85. Bao Z, Xiao H, Liang J, Zhang L, Xiong X, Sun N, Si T, Zhao H. Homology-Integrated CRISPR-Cas (HI-CRISPR) System for One-Step Multigene Disruption in Saccharomyces cerevisiae. ACS Synth Biol. 4 (5): 585-94 (2015).
86. DiCarlo J E, Norville J E, Mali P, Rios X, Aach J, Church G M. Genome engineering in Saccharomyces cerevisiae using CRISPR-Cas systems. Nucleic Acids Res 41:4336-43 (2013).
87. Liu J-J, Kong I I, Zhang G-C, Jayakody L N, Kim H, Xia P-F, Kwak S, Sung B H, Sohn J-H, Walukiewicz H E, Rao C V., Jin Y-S. Metabolic engineering of a probiotic Saccharomyces boulardii. Appl Environ Microbiol. 82 (8): 2280-7 (2016).
88. Shi S, Liang Y, Zhang M M, Ang E L, Zhao H. A highly efficient single-step, markerless strategy for multi-copy chromosomal integration of large biochemical pathways in Saccharomyces cerevisiae. Metab Eng 33:19-27 (2016).

1-67. (canceled)

68. A method of preventing a primary Clostridum difficile (C. difficile) infection or preventing a recurrent C. difficile) infection in a subject, comprising administering an effective amount of an engineered strain of Saccharomyces boulardii yeast to the subject, wherein the engineered strain of Saccharomyces boulardii yeast produces a tetra-specific, tetrameric binding agent comprising: a first, a second, a third, and a fourth linked V_{H}H peptide monomer, wherein the first and third V_{H}H peptide monomers have binding specificity for epitopes of TcdA and comprise the amino acid sequences of AH3 (SEQ ID NO:7) and AA6 (SEQ ID NO:5), respectively, and wherein the second and fourth V_{H}H peptide monomers have binding specificity for epitopes of TcdB and comprise the amino acid sequences 5D (SEQ ID NO:1) and E3 (SEQ ID NO: 3), respectively.

69. The method of claim 68, wherein the C. difficile infection is a primary C. difficile infection.

70. The method of claim 68, wherein the C. difficile infection is a recurrent C. difficile infection.

71. The method of claim 68, wherein the tetra-specific, tetrameric binding agent an amino acid sequence of SEQ ID NO:109.

72. The method of claim 68, wherein the tetra-specific, tetrameric binding agent an amino acid sequence of SEQ ID NO: 19.

73. The method of claim 68, wherein the tetra-specific, tetrameric binding agent an amino acid sequence of SEQ ID NO:107.

74. The method of claim 68, wherein the tetra-specific, tetrameric binding agent further comprises an N-terminal secretion signal selected from SEQ ID NO:99 and SEQ ID NO: 103.

75. The method of claim 68, wherein the engineered strain of Saccharomyces boulardii yeast is administered in an amount between 10 μg/kg and 100 mg/kg per body weight of the subject.

76. The method of claim 68, wherein the engineered strain of Saccharomyces boulardii yeast is administered to the subject orally, nasally, or rectally.

77. The method of claim 68, further comprising administering an antibiotic to the subject.

78. A method of reducing recurrence of a Clostridum difficile (C. difficile) infection in a subject, comprising administering an engineered strain of Saccharomyces boulardii yeast that produces a V_{H}H peptide binding agent comprising a first, second, third, and fourth V_{H}H peptide monomer, wherein each V_{H}H peptide monomer independently has binding specificity for a unique epitope of Clostridium difficile toxin A (TcdA) or toxin B (TcdB).

79. The method of claim 78, wherein the first and third V_{H}H peptide monomers have binding specificity for epitopes of TedA and comprise the amino acid sequences of AH3 (SEQ ID NO: 7) and AA6 (SEQ ID NO:5), respectively, and wherein the second and fourth V_{H}H peptide monomers have binding specificity for epitopes of TcdB and comprise the amino acid sequences 5D (SEQ ID NO:1) and E3 (SEQ ID NO:3), respectively.

80. The method of claim 78, wherein the tetra-specific, tetrameric binding agent an amino acid sequence of SEQ ID NO: 109, 107, or 19.

81. The method of claim 78, wherein the tetra-specific, tetrameric binding agent further comprises an N-terminal secretion signal selected from SEQ ID NO:99 and SEQ ID NO: 103.

82. The method of claim 78, wherein the engineered strain of Saccharomyces boulardii yeast is administered to the subject orally, nasally, or rectally in an amount between 10 ug/kg and 100 mg/kg per body weight of the subject.

83. A method of treating a Clostridum difficile (C. difficile) infection in a subject, comprising administering an engineered strain of Saccharomyces boulardii yeast that produces a V_{H}H peptide binding agent comprising a first, second, third, and fourth V_{H}H peptide monomer, wherein each V_{H}H peptide monomer independently has binding specificity for a unique epitope of Clostridium difficile toxin A (TcdA) or toxin B (TcdB).

84. The method of claim 83, wherein the first and third V_{H}H peptide monomers have binding specificity for epitopes of TedA and comprise the amino acid sequences of AH3 (SEQ ID NO: 7) and AA6 (SEQ ID NO:5), respectively, and wherein the second and fourth V_{H}H peptide monomers have binding specificity for epitopes of TcdB and comprise the amino acid sequences 5D (SEQ ID NO:1) and E3 (SEQ ID NO:3), respectively.

85. The method of claim 83, wherein the tetra-specific, tetrameric binding agent an amino acid sequence of SEQ ID NO: 109, 107, or 19.

86. The method of claim 83, wherein the tetra-specific, tetrameric binding agent further comprises an N-terminal secretion signal selected from SEQ ID NO:99 and SEQ ID NO: 103.

87. The method of claim 83, wherein the engineered strain of Saccharomyces boulardii yeast is administered to the subject orally, nasally, or rectally in an amount between 10 ug/kg and 100 mg/kg per body weight of the subject.


SEQUENCE LISTING (117 sequences)

SEQ ID NO: 1 - protein, 127 aa, synthetic construct
  REGION 1..127: note = Codon-optimized VHH peptide monomer 5D
QVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSS

SEQ ID NO: 2 - DNA, 381 bp, synthetic construct
  misc_feature 1..381: note = Codon-optimized VHH peptide monomer 5D
  source 1..381: mol_type = other DNA
caggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtctcgagc

SEQ ID NO: 3 - protein, 111 aa, synthetic construct
  REGION 1..111: note = Codon-optimized VHH peptide monomer E3
QVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSS

SEQ ID NO: 4 - DNA, 333 bp, synthetic construct
  misc_feature 1..333: note = Codon-optimized VHH peptide monomer E3
  source 1..333: mol_type = other DNA
caagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagt

SEQ ID NO: 5 - protein, 121 aa, synthetic construct
  REGION 1..121: note = Codon-optimized VHH peptide monomer AA6
QLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSS

SEQ ID NO: 6 - DNA, 363 bp, synthetic construct
  misc_feature 1..363: note = Codon-optimized VHH peptide monomer AA6
  source 1..363: mol_type = other DNA
caactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaagtaactgtctcgagc

SEQ ID NO: 7 - protein, 126 aa, synthetic construct
  REGION 1..126: note = Codon-optimized VHH peptide monomer AH3
QVQLVETGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSS

SEQ ID NO: 8 - DNA, 378 bp, synthetic construct
  misc_feature 1..378: note = Codon-optimized VHH peptide monomer AH3
  source 1..378: mol_type = other DNA
caggtacagctggtggagacgggggggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtctcgagt

SEQ ID NO: 9 - protein, 15 aa, synthetic construct
  REGION 1..15: note = Codon-optimized flexible linker 1
GGGGSGGGGSGGGGS

SEQ ID NO: 10 - DNA, 45 bp, synthetic construct
  misc_feature 1..45: note = Codon-optimized flexible linker 1
  source 1..45: mol_type = other DNA
ggcggtggagggtctggtgggggaggctcagggggtggaggcagc

SEQ ID NO: 11 - protein, 16 aa, synthetic construct
  REGION 1..16: note = Codon-optimized flexible linker 2
GGGSGGGSGGGSGGGS

SEQ ID NO: 12 - DNA, 48 bp, synthetic construct
  misc_feature 1..48: note = Codon-optimized flexible linker 2
  source 1..48: mol_type = other DNA
ggtggcggaagcggagggggcagcgggggtgggagcggtgggggcagc

SEQ ID NO: 13 - protein, 15 aa, synthetic construct
  REGION 1..15: note = Codon-optimized flexible linker 3
GGGGSGGGGSGGGGS

SEQ ID NO: 14 - DNA, 45 bp, synthetic construct
  misc_feature 1..45: note = Codon-optimized flexible linker 3
  source 1..45: mol_type = other DNA
gggggaggcggttcaggcggtgggggatctggcgggggtggatcc

SEQ ID NO: 15 - protein, 269 aa, synthetic construct
  REGION 1..269: note = AH3-5D heterodimer
QVQLVETGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSGGGGSGGGGSGGGGSQVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSS

SEQ ID NO: 16 - DNA, 807 bp, synthetic construct
  misc_feature 1..807: note = AH3-5D heterodimer
  source 1..807: mol_type = other DNA
caggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtctcgagc

SEQ ID NO: 17 - protein, 247 aa, synthetic construct
  REGION 1..247: note = AA6-E3 heterodimer
QLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSGGGGSGGGGSGGGGSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSS

SEQ ID NO: 18 - DNA, 741 bp, synthetic construct
  misc_feature 1..741: note = AA6-E3 heterodimer
  source 1..741: mol_type = other DNA
caactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagt

SEQ ID NO: 19 - protein, 532 aa, synthetic construct
  REGION 1..532: note = ABAB binding agent
QVQLVETGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSGGGGSGGGGSGGGGSQVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSGGGSGGGSGGGSGGGSQLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSGGGGSGGGGSGGGGSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSS

SEQ ID NO: 20 - DNA, 1596 bp, synthetic construct
  misc_feature 1..1596: note = ABAB binding agent
  source 1..1596: mol_type = other DNA
caggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagt

SEQ ID NO: 21 - protein, 12 aa, synthetic construct
  REGION 1..12: note = Albumin-binding peptide
DICLPRWGCLWD

SEQ ID NO: 22 - protein, 761 aa, synthetic construct
  REGION 1..761: note = ABAB-Fc binding agent
QVQLVETGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSGGGGSGGGGSGGGGSQVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSGGGSGGGSGGGSGGGSQLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSGGGGSGGGGSGGGGSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 2286 bp, synthetic construct
  misc_feature 1..2286: note = ABAB-Fc binding agent
  source 1..2286: mol_type = other DNA
caggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 24 - protein, 356 aa, synthetic construct
  REGION 1..356: note = 5D-Fc binding agent
QVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 1071 bp, synthetic construct
  misc_feature 1..1071: note = 5D-Fc binding agent
  source 1..1071: mol_type = other DNA
caggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtctcgagcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - protein, 340 aa, synthetic construct
  REGION 1..340: note = E3-Fc binding agent
QVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1023 bp, synthetic construct
  misc_feature 1..1023: note = E3-Fc binding agent
  source 1..1023: mol_type = other DNA
caagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 28 - protein, 350 aa, synthetic construct
  REGION 1..350: note = AA6-Fc binding agent
QLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1053 bp, synthetic construct
  misc_feature 1..1053: note = AA6-Fc binding agent
  source 1..1053: mol_type = other DNA
caactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaagtaactgtctcgagcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 30 - protein, 356 aa, synthetic construct
  REGION 1..356: note = AH3-Fc binding agent
QVQLVETGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 1071 bp, synthetic construct
  misc_feature 1..1071: note = AH3-Fc binding agent
  source 1..1071: mol_type = other DNA
caggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtctcgagcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 32 - protein, 498 aa, synthetic construct
  REGION 1..498: note = AH3-5D-Fc binding agent
QVQLVETGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSGGGGSGGGGSGGGGSQVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 1497 bp, synthetic construct
  misc_feature 1..1497: note = AH3-5D-Fc binding agent
  source 1..1497: mol_type = other DNA
caggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtctcgagcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 34 - protein, 476 aa, synthetic construct
  REGION 1..476: note = AA6-E3-Fc binding agent
QLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSGGGGSGGGGSGGGGSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1431 bp, synthetic construct
  misc_feature 1..1431: note = AA6-E3-Fc binding agent
  source 1..1431: mol_type = other DNA
caactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 36 - protein, 457 aa, synthetic construct
  REGION 1..457: note = AH3-IgG1-heavy chain
QVQLVETGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 1374 bp, synthetic construct
  misc_feature 1..1374: note = AH3-IgG1-heavy chain
  source 1..1374: mol_type = other DNA
caggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtctcgagtgcgtcgaccaagggcccatcggtcttcccgctagcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 38 - protein, 457 aa, synthetic construct
  REGION 1..457: note = 5D-IgG1-heavy chain
QVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 1374 bp, synthetic construct
  misc_feature 1..1374: note = 5D-IgG1-heavy chain
  source 1..1374: mol_type = other DNA
caggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtctcgagcgcgtcgaccaagggcccatcggtcttcccgctagcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 40 - protein, 228 aa, synthetic construct
  REGION 1..228: note = AA6-IgG1-kappa chain
QLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 687 bp, synthetic construct
  misc_feature 1..687: note = AA6-IgG1-kappa chain
  source 1..687: mol_type = other DNA
caactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaagtaactgtctcgagccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 42 - protein, 218 aa, synthetic construct
  REGION 1..218: note = E3-IgG1-kappa chain
QVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - DNA, 657 bp, synthetic construct
  misc_feature 1..657: note = E3-IgG1-kappa chain
  source 1..657: mol_type = other DNA
caagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 44 - protein, 599 aa, synthetic construct
  REGION 1..599: note = AH3-5D-IgG1 heavy chain
QVQLVETGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSGGGGSGGGGSGGGGSQVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 1800 bp, synthetic construct
  misc_feature 1..1800: note = AH3-5D-IgG1 heavy chain
  source 1..1800: mol_type = other DNA
caggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtctcgagcgcgtcgaccaagggcccatcggtcttcccgctagcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 46 - protein, 354 aa, synthetic construct
  REGION 1..354: note = AA6-E3-IgG1 light chain
QLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSGGGGSGGGGSGGGGSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - DNA, 1065 bp, synthetic construct
  misc_feature 1..1065: note = AA6-E3-IgG1 light chain
  source 1..1065: mol_type = other DNA
caactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 48 - protein, 127 aa, synthetic construct; identical to SEQ ID NO: 1
  REGION 1..127: note = VHH peptide monomer 5D

SEQ ID NO: 49 - DNA, 381 bp, synthetic construct
  misc_feature 1..381: note = VHH peptide monomer 5D
  source 1..381: mol_type = other DNA
caggtgcagctcgtggagtcaggtggaggcttggtgcagcctggggggtctctgagactctcctgtgaagcctctggattcactttggattattatggtataggctggttccgccagcccccagggaaggagcgcgaggcggtctcatatattagtgccagtgcccgtacgatattgtatgcagattccgtgaagggccgatttaccatctccagagacaatgccaagaacgcggtgtatctacaaatgaacagcctgaaacgtgaggacacggctgtctattactgtgcgaggcggcgattctccgcgtctagtgttaatagatggcttgccgacgactatgacgtctggggtcgggggacccaggtcgcggtgtcctca

SEQ ID NO: 50 - protein, 111 aa, synthetic construct; identical to SEQ ID NO: 3
  REGION 1..111: note = VHH peptide monomer E3

SEQ ID NO: 51 - DNA, 333 bp, synthetic construct
  misc_feature 1..333: note = VHH peptide monomer E3
  source 1..333: mol_type = other DNA
caggtgcagctcgtggagtcgggcggaggcttggtgcagactggggggtctctgagactctcctgtgcatcctctggaagtatcgccggtttcgaaaccgtgacctggtcccgccaggctcctggaaagtcgctccagtgggtcgcatcgatgactaaaactaataacgagatctattcagactccgtgaagggccgattcatcatctccagagacaacgccaagaatacggtgtatctacaaatgaacagcctgaaacctgaggacacaggcgtctatttttgtaaaggtcctgagttgaggggccaggggatccaggtcaccgtctcctcg

SEQ ID NO: 52 - protein, 121 aa, synthetic construct; identical to SEQ ID NO: 5
  REGION 1..121: note = VHH peptide monomer AA6

SEQ ID NO: 53 - DNA, 363 bp, synthetic construct
  misc_feature 1..363: note = VHH peptide monomer AA6
  source 1..363: mol_type = other DNA
cagttgcagctcgtggagacagggggaggcttggtgcagcctggggggtctctgagactctcctgtgcagcctctggattcacgttcagtgactacgtcatgacctgggtccgccaggctccaggaaaggggcccgaatggatcgcaactattaatacggacggtagcacgatgcgtgatgactccacaaaaggccgattcaccatctccagagacaacgccaagaacacactgtatctgcaaatgaccagcctgaaaccggaggacacggccctgtattactgtgcgagaggccgcgtgatctccgcctccgcgataagaggggcggttaggggcccggggacccaggtcaccgtctcctca

SEQ ID NO: 54 - protein, 126 aa, synthetic construct; identical to SEQ ID NO: 7
  REGION 1..126: note = VHH peptide monomer AH3

SEQ ID NO: 55 - DNA, 378 bp, synthetic construct
  misc_feature 1..378: note = VHH peptide monomer AH3
  source 1..378: mol_type = other DNA
caggtgcagctcgtggagacggggggcttggtgcagcctggggggtctctgagactctcctgtgcagcctctggattcactttggattattcgtccataggctggttccgccaggccccagggaaggagcgtgagggggtctcatgtattagtagtagtggtgatagcacaaagtatgcagactccgtgaagggccgattcaccacctccagagacaacgccaagaacacggtgtatctgcaaatgaacagcctgaaacctgacgacacagccgtttattactgtgcagcttttagggcgactatgtgcggcgtgttcccccttagcccctacggcaaggacgactggggcaaagggaccctggtcaccgtctcctca

SEQ ID NO: 56 - protein, 15 aa, synthetic construct
  REGION 1..15: note = Flexible linker 1
GGGGSGGGGSGGGGS

SEQ ID NO: 57 - DNA, 45 bp, synthetic construct
  misc_feature 1..45: note = Flexible linker 1
  source 1..45: mol_type = other DNA
ggcggtggtggctctggtggcggcggttccggtggcggtggcagc

SEQ ID NO: 58 - protein, 15 aa, synthetic construct
  REGION 1..15: note = Flexible linker 2
GGGGSGGGGSGGGGS

SEQ ID NO: 59 - DNA, 45 bp, synthetic construct
  misc_feature 1..45: note = Flexible linker 2
  source 1..45: mol_type = other DNA
ggtggaggcggttcaggcggaggtggctctggcggtggcggttcc

SEQ ID NO: 60 - protein, 15 aa, synthetic construct
  REGION 1..15: note = Flexible linker 3
GGGGSGGGGSGGGGS

SEQ ID NO: 61 - DNA, 45 bp, synthetic construct; identical to SEQ ID NO: 57
  misc_feature 1..45: note = Flexible linker 3
  source 1..45: mol_type = other DNA

SEQ ID NO: 62 - protein, 259 aa, synthetic construct
  REGION 1..259: note = 5D-E3 heterodimer
QVQLVESGGGLVQPGGSLRLSCEASGFTLDYYGIGWFRQPPGKEREAVSYISASARTILYADSVKGRFTISRDNAKNAVYLQMETNSLKREDTAVYYCARRRFSASSVNRWLADDYDVWGRGTQVAVSSGGGGSGGGGSGGGGSQVQLVESGGGLVQTGGSLRLSCASSGSIAGFETVTWSRQAPGKSLQWVASMETTKTNNEIYSDSVKGRFIISRDNAKNTVYLQMETNSLKPEDTGVYFCKGPELRGQGIQVTVSS

SEQ ID NO: 63 - DNA, 759 bp, synthetic construct
  misc_feature 1..759: note = 5D-E3 heterodimer
  source 1..759: mol_type = other DNA
caggtgcagctcgtggagtcaggtggaggcttggtgcagcctggggggtctctgagactctcctgtgaagcctctggattcactttggattattatggtataggctggttccgccagcccccagggaaggagcgcgaggcggtctcatatattagtgccagtgcccgtacgatattgtatgcagattccgtgaagggccgatttaccatctccagagacaatgccaagaacgcggtgtatctacaaatgaacagcctgaaacgtgaggacacggctgtctattactgtgcgaggcggcgattctccgcgtctagtgttaatagatggcttgccgacgactatgacgtctggggtcgggggacccaggtcgcggtgtcctcaggcggtggtggctctggtggcggcggttccggtggcggtggcagccaggtgcagctcgtggagtcgggcggaggcttggtgcagactggggggtctctgagactctcctgtgcatcctctggaagtatcgccggtttcgaaaccgtgacctggtcccgccaggctcctggaaagtcgctccagtgggtcgcatcgatgactaaaactaataacgagatctattcagactccgtgaagggccgattcatcatctccagagacaacgccaagaatacggtgtatctacaaatgaacagcctgaaacctgaggacacaggcgtctatttttgtaaaggtcctgagttgaggggccaggggatccaggtcaccgtctcctcg

SEQ ID NO: 64 - protein, 272 aa, synthetic construct
  REGION 1..272: note = AH3-AA6 heterodimer
QVQLVETGGLVQPGGSLRLSCAASGFTLDYSSIGWFRQAPGKEREGVSCISSSGDSTKYADSVKGRFTTSRDNAKNTVYLQMETNSLKPDDTAVYYCAAFRATMETCGVFPLSPYGKDDWGKGTLVTVSSGGGGSGGGGSGGGGSQLQLVETGGGLVQPGGSLRLSCAASGFTFSDYVMETTWVRQAPGKGPEWIATINTDGSTMETRDDSTKGRFTISRDNAKNTLYLQMETTSLKPEDTALYYCARGRVISASAIRGAVRGPGTQVTVSS

SEQ ID NO: 65 - DNA, 786 bp, synthetic construct
  misc_feature 1..786: note = AH3-AA6 heterodimer
  source 1..786: mol_type = other DNA
caggtgcagctcgtggagacggggggcttggtgcagcctggggggtctctgagactctcctgtgcagcctctggattcactttggattattcgtccataggctggttccgccaggccccagggaaggagcgtgagggggtctcatgtattagtagtagtggtgatagcacaaagtatgcagactccgtgaagggccgattcaccacctccagagacaacgccaagaacacggtgtatctgcaaatgaacagcctgaaacctgacgacacagccgtttattactgtgcagcttttagggcgactatgtgcggcgtgttcccccttagcccctacggcaaggacgactggggcaaagggaccctggtcaccgtctcctcaggcggtggtggctctggtggcggcggttccggtggcggtggcagccagttgcagctcgtggagacagggggaggcttggtgcagcctggggggtctctgagactctcctgtgcagcctctggattcacgttcagtgactacgtcatgacctgggtccgccaggctccaggaaaggggcccgaatggatcgcaactattaatacggacggtagcacgatgcgtgatgactccacaaaaggccgattcaccatctccagagacaacgccaagaacacactgtatctgcaaatgaccagcctgaaaccggaggacacggccctgtattactgtgcgagaggccgcgtgatctccgcctccgcgataagaggggcggttaggggcccggggacccaggtcaccgtctcctca

SEQ ID NO: 66 - protein, 6 aa, synthetic construct
  REGION 1..6: note = six-histidine tag
HHHHHH

SEQ ID NO: 67 - protein, 13 aa, synthetic construct
  REGION 1..13: note = E-tag for protein purification
GAPVPYPDPLEPR

SEQ ID NO: 68 - DNA, 6985 bp, synthetic construct
  misc_feature 1..6985: note = Plasmid pD1214-FAKS-His-hABAB-D7
  source 1..6985: mol_type = other DNA
tcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggggtcgtgcaggtatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagtgaccgcctcgtttctttttcttcgtcgaaaaaggcaataaaaatttttatcacgtttctttttcttgaaaatttttttttttgatttttttctctttcgatgacctcccattgatatttaagttaataaacggacttcaatttctcaagtttcagtttcatttttcttgttctattacaactttttttacttcttgctcattagaaagaaagcatagcaatctaatctaagtttaaatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatgcatcatcatcatcatcatcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagtgctcctacaaaagccaaacggagagtggtccagagagagaagacctaataaggttgaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcccctcacctgcacgcaaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacgctatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagtcgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgatattgg

SEQ ID NO: 69 - DNA, 6916 bp, synthetic construct
  misc_feature 1..6916: note = Plasmid pD1214-FAKS-hABAB
  source 1..6916: mol_type = other DNA
tcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggggtcgtgcaggtatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagtgaccgcctcgtttctttttcttcgtcgaaaaaggcaataaaaatttttatcacgtttctttttcttgaaaatttttttttttgatttttttctctttcgatgacctcccattgatatttaagttaataaacggacttcaatttctcaagtttcagtttcatttttcttgttctattacaactttttttacttcttgctcattagaaagaaagcatagcaatctaatctaagtttaaatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagtggttgaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcccctcacctgcacgcaaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacgctatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagtcgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgatattgg

SEQ ID NO: 70 - DNA, 6833 bp, synthetic construct
  misc_feature 1..6833: note = Plasmid pD1214-AKS-hABAB
  source 1..6833: mol_type = other DNA
ggttaaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcccctcacctgcacgcaaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacgctatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagtcgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgatattggtcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggggtcgtgcaggtatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagtgaccgcctcgtttctttttcttcgtcgaaaaaggcaataaaaatttttatcacgtttctttttcttgaaaatttttttttttgatttttttctctttcgatgacctcccattgatatttaagttaataaacggacttcaatttctcaagtttcagtttcatttttcttgttctattacaactttttttacttcttgctcattagaaagaaagcatagcaatctaatctaagtttaaaatgagatttccttcaatttttactgctgttttattcgcagcatcctccgcattagctgctccagtcaacactacaacagaagatgaattagaaggggatttcgatgttgctgttttgccattttccgccagcattgctgctaaagaagaaggggtatctctcgagaaaagagaggctgaagccatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagt

SEQ ID NO: 71 - DNA, 6821 bp, synthetic construct
  misc_feature 1..6821: note = Plasmid pD1214-AK-hABAB
  source 1..6821: mol_type = other DNA
ggttaaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcccctcacctgcacgcaaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacgctatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagtcgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgatattggtcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggggtcgtgcaggtatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagtgaccgcctcgtttctttttcttcgtcgaaaaaggcaataaaaatttttatcacgtttctttttcttgaaaatttttttttttgatttttttctctttcgatgacctcccattgatatttaagttaataaacggacttcaatttctcaagtttcagtttcatttttcttgttctattacaactttttttacttcttgctcattagaaagaaagcatagcaatctaatctaagtttaaaatgagatttccttcaatttttactgctgttttattcgcagcatcctccgcattagctgctccagtcaacactacaacagaagatgaattagaaggggatttcgatgttgctgttttgccattttccgccagcattgctgctaaagaagaaggggtatctctcgagaaaagaatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagt

SEQ ID NO: 72 - DNA, 6707 bp, synthetic construct
  misc_feature 1..6707: note = Plasmid pD1214-AT-hABAB
  source 1..6707: mol_type = other DNA
ggttaaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcccctcacctgcacgcaaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacgctatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagtcgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgatattggtcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggggtcgtgcaggtatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagtgaccgcctcgtttctttttcttcgtcgaaaaaggcaataaaaatttttatcacgtttctttttcttgaaaatttttttttttgatttttttctctttcgatgacctcccattgatatttaagttaataaacggacttcaatttctcaagtttcagtttcatttttcttgttctattacaactttttttacttcttgctcattagaaagaaagcatagcaatctaatctaagtttaaaatgagatttccttcaatttttactgctgttttattcgcagcatcctccgcattagctatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagt

SEQ ID NO: 73 - DNA, 6710 bp, synthetic construct
  misc_feature 1..6710: note = Plasmid pD1214-AA-hABAB
  source 1..6710: mol_type = other DNA
ggttaaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcccctcacctgcacgcaaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacgctatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagtcgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgatattggtcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggggtcgtgcaggtatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagtgaccgcctcgtttctttttcttcgtcgaaaaaggcaataaaaatttttatcacgtttctttttcttgaaaatttttttttttgatttttttctctttcgatgacctcccattgatatttaagttaataaacggacttcaatttctcaagtttcagtttcatttttcttgttctattacaactttttttacttcttgctcattagaaagaaagcatagcaatctaatctaagtttaaaatggttgcctggtggtcccttttcctgtacggtttacaagtcgctgctcctgcattggcaatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagt

SEQ ID NO: 74 - DNA, 6704 bp, synthetic construct
  misc_feature 1..6704: note = Plasmid pD1214-GA-hABAB
  source 1..6704: mol_type = other DNA
ggttaaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcccctcacctgcacgcaaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacgctatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagtcgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgatattggtcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggggtcgtgcaggtatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagtgaccgcctcgtttctttttcttcgtcgaaaaaggcaataaaaatttttatcacgtttctttttcttgaaaatttttttttttgatttttttctctttcgatgacctcccattgatatttaagttaataaacggacttcaatttctcaagtttcagtttcatttttcttgttctattacaactttttttacttcttgctcattagaaagaaagcatagcaatctaatctaagtttaaaatgtcttttagatcacttttagctctgtccggtttggtttgtagtggattggcaatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagt

SEQ ID NO: 75 - DNA, 6698 bp, synthetic construct
  misc_feature 1..6698: note = Plasmid pD1214-IN-hABAB
  source 1..6698: mol_type = other DNA
ggttaaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcccctcacctgcacgcaaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacgctatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagtcgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgatattggtcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggggtcgtgcaggtatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagtgaccgcctcgtttctttttcttcgtcgaaaaaggcaataaaaatttttatcacgtttctttttcttgaaaatttttttttttgatttttttctctttcgatgacctcccattgatatttaagttaataaacggacttcaatttctcaagtttcagtttcatttttcttgttctattacaactttttttacttcttgctcattagaaagaaagcatagcaatctaatctaagtttaaaatgaaacttgcttactctctgttgttaccattggccggtgtttccgcaatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagt

SEQ ID NO: 76 - DNA, 6707 bp, synthetic construct
  misc_feature 1..6707: note = Plasmid pD1214-IVS-hABAB
  source 1..6707: mol_type = other DNA
ggttaaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcccctcacctgcacgcaaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacgctatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagtcgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgatattggtcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggggtcgtgcaggtatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagtgaccgcctcgtttctttttcttcgtcgaaaaaggcaataaaaatttttatcacgtttctttttcttgaaaatttttttttttgatttttttctctttcgatgacctcccattgatatttaagttaataaacggacttcaatttctcaagtttcagtttcatttttcttgttctattacaactttttttacttcttgctcattagaaagaaagcatagcaatctaatctaagtttaaaatgttacttcaagctttcttgttcctgttggctggttttgccgcaaaaatctccgcaatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagt

SEQ ID NO: 77 - DNA, 6728 bp, synthetic construct
  misc_feature 1..6728: note = Plasmid pD1214-KP-hABAB
  source 1..6728: mol_type = other DNA
ggttaaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcccctcacctgcacgcaaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacgctatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagtcgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgatattggtcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggggtcgtgcaggtatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagtgaccgcctcgtttctttttcttcgtcgaaaaaggcaataaaaatttttatcacgtttctttttcttgaaaatttttttttttgatttttttctctttcgatgacctcccattgatatttaagttaataaacggacttcaatttctcaagtttcagtttcatttttcttgttctattacaactttttttacttcttgctcattagaaagaaagcatagcaatctaatctaagtttaaaatgacaaaaccaacccaagtcttggtgagatctgtatccattcttttctttatcactttattgcatctggttgttgctatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagt

SEQ ID NO: 78 - DNA, 6728 bp, synthetic construct
  misc_feature 1..6728: note = Plasmid pD1214-LZ-hABAB
  source 1..6728: mol_type = other DNA
ggttaaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcccctcacctgcacgcaaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacgctatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagtcgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgatattggtcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggggtcgtgcaggtatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagtgaccgcctcgtttctttttcttcgtcgaaaaaggcaataaaaatttttatcacgtttctttttcttgaaaatttttttttttgatttttttctctttcgatgacctcccattgatatttaagttaataaacggacttcaatttctcaagtttcagtttcatttttcttgttctattacaactttttttacttcttgctcattagaaagaaagcatagcaatctaatctaagtttaaaatgttaggtaaaaatgatccaatgtgtctggtgcttgttctgttgggtttgactgctctattgggtatctgccaaggaatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagt

SEQ ID NO: 79 - DNA, 6704 bp, synthetic construct
  misc_feature 1..6704: note = Plasmid pD1214-SA-hABAB
  source 1..6704: mol_type = other DNA
ggttaaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcccctcacctgcacgcaaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacgctatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagtcgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgatattggtcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggggtcgtgcaggtatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagtgaccgcctcgtttctttttcttcgtcgaaaaaggcaataaaaatttttatcacgtttctttttcttgaaaatttttttttttgatttttttctctttcgatgacctcccattgatatttaagttaataaacggacttcaatttctcaagtttcagtttcatttttcttgttctattacaactttttttacttcttgctcattagaaagaaagcatagcaatctaatctaagtttaaaatgaaatgggttacttttatctcacttttgttcctgttctccagcgcttactctatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagt

SEQ ID NO: 80 - DNA, 6574 bp, synthetic construct
  misc_feature 1..6574: note = Plasmid pCEV-G4-Km
  source 1..6574: mol_type = other DNA
ggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagttttttgattaaaattaaaaaaactttttgtttttgtgtttattctttgttcttagaaaagacaagttgagcttgtttgttcttgatgttttattattttacaatagctgcaaatgaagaatagattcgaacattgtgaagtattggcatatatcgtctctatttatactttttttttttcagttctagtatattttgtattttcctccttttcattctttcagttgccaataagttacaggggatctcgaaagatggtggggatttttccttgaaagacgactttttgccatctaatttttccttgttgcctctgaaaattatccagcagaagcaaatgtaaaagatgaacctcagaagaacacgcaggggcccgaaattgttcctacgagaagtagtgggtcataaaaagtttattccctggaaaaaaaattttgcgttgcctttctggagaattttttcgaattagcgtgctgccactgcatgcatttctgagaagtgtgggcattcttccaccagttgttcctcctaaaaaaaaaaagatttcctaccccgcacaaattcctgcatacccctcatttccacggggccggccgcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaggatccgtaatacgactcactatagggcccgggcgtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatggtagacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccacta

SEQ ID NO: 81 - DNA, 8211 bp, synthetic construct
  misc_feature 1..8211: note = Plasmid pCEV-G4-Km-TEF-AT-hABAB*
  source 1..8211: mol_type = other DNA
gcctaggatgagatttccttcaatttttactgctgttttattcgcagcatcctccgcattagctggcgcccacgtgatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagtgtcgacggtacctaagctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatggtagacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccactaggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagttttttgattaaaattaaaaaaactttttgtttttgtgtttattctttgttcttagaaaagacaagttgagcttgtttgttcttgatgttttattattttacaatagctgcaaatgaagaatagattcgaacattgtgaagtattggcatatatcgtctctatttatactttttttttttcagttctagtatattttgtattttcctccttttcattctttcagttgccaataagttacaggggatctcgaaagatggtggggatttttccttgaaagacgactttttgccatctaatttttccttgttgcctctgaaaattatccagcagaagcaaatgtaaaagatgaacctcagaagaacacgcaggggcccgaaattgttcctacgagaagtagtgggtcataaaaagtttattccctggaaaaaaaattttgcgttgcctttctggagaattttttcgaattagcgtgctgccactgcatgcatttctgagaagtgtgggcattcttccaccagttgttcctcctaaaaaaaaaaagatttcctaccccgcacaaattcctgcatacccctcatttccacggggccggccgcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaggatccgtaatacgactcactatagggcccgggcgtcga

SEQ ID NO: 82 - DNA, 8202 bp, synthetic construct
  misc_feature 1..8202: note = Plasmid pCEV-G4-Km-TEF-IVS-hABAB*
  source 1..8202: mol_type = other DNA
catgttacttcaagctttcttgttcctgttggctggttttgccgcaaaaatctccgcaggcgcccacgtgatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagttaaggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatggtagacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccactaggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagttttttgattaaaattaaaaaaactttttgtttttgtgtttattctttgttcttagaaaagacaagttgagcttgtttgttcttgatgttttattattttacaatagctgcaaatgaagaatagattcgaacattgtgaagtattggcatatatcgtctctatttatactttttttttttcagttctagtatattttgtattttcctccttttcattctttcagttgccaataagttacaggggatctcgaaagatggtggggatttttccttgaaagacgactttttgccatctaatttttccttgttgcctctgaaaattatccagcagaagcaaatgtaaaagatgaacctcagaagaacacgcaggggcccgaaattgttcctacgagaagtagtgggtcataaaaagtttattccctggaaaaaaaattttgcgttgcctttctggagaattttttcgaattagcgtgctgccactgcatgcatttctgagaagtgtgggcattcttccaccagttgttcctcctaaaaaaaaaaagatttcctaccccgcacaaattcctgcatacccctcatttccacggggccggccgcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaggatccgtaatacgactcactatagggcccgggcgtcga

SEQ ID NO: 83 - DNA, 8190 bp, synthetic construct
  misc_feature 1..8190: note = Plasmid pCEV-G4-Km-TEF-AT-hABAB
  source 1..8190: mol_type = other DNA
catgagatttccttcaatttttactgctgttttattcgcagcatcctccgcattagctatgcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagttaaggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatggtagacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccactaggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagttttttgattaaaattaaaaaaactttttgtttttgtgtttattctttgttcttagaaaagacaagttgagcttgtttgttcttgatgttttattattttacaatagctgcaaatgaagaatagattcgaacattgtgaagtattggcatatatcgtctctatttatactttttttttttcagttctagtatattttgtattttcctccttttcattctttcagttgccaataagttacaggggatctcgaaagatggtggggatttttccttgaaagacgactttttgccatctaatttttccttgttgcctctgaaaattatccagcagaagcaaatgtaaaagatgaacctcagaagaacacgcaggggcccgaaattgttcctacgagaagtagtgggtcataaaaagtttattccctggaaaaaaaattttgcgttgcctttctggagaattttttcgaattagcgtgctgccactgcatgcatttctgagaagtgtgggcattcttccaccagttgttcctcctaaaaaaaaaaagatttcctaccccgcacaaattcctgcatacccctcatttccacggggccggccgcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaggatccgtaatacgactcactatagggcccgggcgtcga

SEQ ID NO: 84 - DNA, 8190 bp, synthetic construct
  misc_feature 1..8190: note = Plasmid pCEV-G4-Km-TEF-AT-yABAB
  source 1..8190: mol_type = other DNA
ggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagttttttgattaaaattaaaaaaactttttgtttttgtgtttattctttgttcttagaaaagacaagttgagcttgtttgttcttgatgttttattattttacaatagctgcaaatgaagaatagattcgaacattgtgaagtattggcatatatcgtctctatttatactttttttttttcagttctagtatattttgtattttcctccttttcattctttcagttgccaataagttacaggggatctcgaaagatggtggggatttttccttgaaagacgactttttgccatctaatttttccttgttgcctctgaaaattatccagcagaagcaaatgtaaaagatgaacctcagaagaacacgcaggggcccgaaattgttcctacgagaagtagtgggtcataaaaagtttattccctggaaaaaaaattttgcgttgcctttctggagaattttttcgaattagcgtgctgccactgcatgcatttctgagaagtgtgggcattcttccaccagttgttcctcctaaaaaaaaaaagatttcctaccccgcacaaattcctgcatacccctcatttccacggggccggccgcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaggatccgtaatacgactcactatagggcccgggcgtcgacatgagatttccttcaatttttactgctgttttattcgcagcatcctccgcattagctatgcaagtacaattggttgaaaccggtggtggtttagttcaaccaggtggtagtttgagattatcttgtgctgcatcaggttttacattggattattcttcaataggttggttcagacaagctcctggtaaagaaagagaaggtgtttcttgcatatccagttctggtgactcaactaaatatgctgactccgttaagggtagattcactacttcaagagataacgctaaaaatacagtctacttgcaaatgaactcattaaagccagatgacacagcagtctattactgtgccgcttttagagccaccatgtgcggtgtattcccattgtctccttacggtaaagatgactggggtaaaggtactttagttactgtctcatccggtggtggtggttccggtggtggtggtagtggtggtggtggttctcaagttcaattagtagaatccggtggtggtttagtccaacctggtggtagtttaagattatcctgcgaagcaagtggttttacattagattattacggtatcggttggtttagacaaccacctggtaaagaaagagaagctgtctcttatatttccgctagtgcaagaactatattgtacgcagattctgtaaagggtagattcacaatttcaagagacaatgccaagaacgctgtttatttgcaaatgaactctttgaagagagaagacaccgcagtttattactgtgccagaagaagattttctgcttcttcagtcaacagatggttagcagacgattatgatgtttggggtagaggtacacaagtcgccgtaagttctggtggtggttccggtggtggtagtggtggtggttctggtggtggttcacaattgcaattagtagaaactggtggtggtttggttcaaccaggtggttccttgagattaagttgtgctgcatctggttttactttctctgattacgttatgacatgggtcagacaagctccaggtaaaggtcctgaatggatcgctacaattaataccgacggttccacaatgagagatgacagtaccaagggtagattcactatttcaagagataacgctaagaacacattgtacttacaaatgacctctttgaaaccagaagacaccgcattatattactgtgccagaggtagagtcatatccgccagtgctatcagaggtgcagtaagaggtcctggtactcaagttacagtctcttcaggtggcggcggtagtggcggcggcggttctggcggtggtggttcacaagtccaattggtagaatctggtggtggtttagttcaaactggtggttcattgagattatcctgcgcttccagtggttccattgcaggtttcgaaactgttacatggtcaagacaagctccaggtaaatctttgcaatgggtcgcctcaatgaccaagactaacaacgaaatctattctgattcagttaagggtagattcattatttcaagagataatgctaaaaacaccgtttatttgcaaatgaactcattgaagccagaagatactggtgtttacttctgcaagggtcctgaattaagaggtcaaggtattcaagtaacagtttcttcataaggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatggtagacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccacta

SEQ ID NO: 85 - DNA, 8202 bp, synthetic construct
  misc_feature 1..8202: note = Plasmid pCEV-G4-Km-TEF-X40-AT-yABAB
  source 1..8202: mol_type = other DNA
ggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagttttttgattaaaattaaaaaaactttttgtttttgtgtttattctttgttcttagaaaagacaagttgagcttgtttgttcttgatgttttattattttacaatagctgcaaatgaagaatagattcgaacattgtgaagtattggcatatatcgtctctatttatactttttttttttcagttctagtatattttgtattttcctccttttcattctttcagttgccaataagttacaggggatctcgaaagatggtggggatttttccttgaaagacgactttttgccatctaatttttccttgttgcctctgaaaattatccagcagaagcaaatgtaaaagatgaacctcagaagaacacgcaggggcccgaaattgttcctacgagaagtagtgggtcataaaaagtttattccctggaaaaaaaattttgcgttgcctttctggagaattttttcgaattagcgtgctgccactgcatgcatttctgagaagtgtgggcattcttccaccagttgttcctcctaaaaaaaaaaagatttcctaccccgcacaaattcctgcatacccctcatttccacggggccggccgcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaggatccatgagatttccttcaatttttactgctgttttattcgcagcatcctccgcattagctatgcaagtacaattggttgaaaccggtggtggtttagttcaaccaggtggtagtttgagattatcttgtgctgcatcaggttttacattggattattcttcaataggttggttcagacaagctcctggtaaagaaagagaaggtgtttcttgcatatccagttctggtgactcaactaaatatgctgactccgttaagggtagattcactacttcaagagataacgctaaaaatacagtctacttgcaaatgaactcattaaagccagatgacacagcagtctattactgtgccgcttttagagccaccatgtgcggtgtattcccattgtctccttacggtaaagatgactggggtaaaggtactttagttactgtctcatccggtggtggtggttccggtggtggtggtagtggtggtggtggttctcaagttcaattagtagaatccggtggtggtttagtccaacctggtggtagtttaagattatcctgcgaagcaagtggttttacattagattattacggtatcggttggtttagacaaccacctggtaaagaaagagaagctgtctcttatatttccgctagtgcaagaactatattgtacgcagattctgtaaagggtagattcacaatttcaagagacaatgccaagaacgctgtttatttgcaaatgaactctttgaagagagaagacaccgcagtttattactgtgccagaagaagattttctgcttcttcagtcaacagatggttagcagacgattatgatgtttggggtagaggtacacaagtcgccgtaagttctggtggtggttccggtggtggtagtggtggtggttctggtggtggttcacaattgcaattagtagaaactggtggtggtttggttcaaccaggtggttccttgagattaagttgtgctgcatctggttttactttctctgattacgttatgacatgggtcagacaagctccaggtaaaggtcctgaatggatcgctacaattaataccgacggttccacaatgagagatgacagtaccaagggtagattcactatttcaagagataacgctaagaacacattgtacttacaaatgacctctttgaaaccagaagacaccgcattatattactgtgccagaggtagagtcatatccgccagtgctatcagaggtgcagtaagaggtcctggtactcaagttacagtctcttcaggtggcggcggtagtggcggcggcggttctggcggtggtggttcacaagtccaattggtagaatctggtggtggtttagttcaaactggtggttcattgagattatcctgcgcttccagtggttccattgcaggtttcgaaactgttacatggtcaagacaagctccaggtaaatctttgcaatgggtcgcctcaatgaccaagactaacaacgaaatctattctgattcagttaagggtagattcattatttcaagagataatgctaaaaacaccgtttatttgcaaatgaactcattgaagccagaagatactggtgtttacttctgcaagggtcctgaattaagaggtcaaggtattcaagtaacagtttcttcagtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatggtagacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccacta

SEQ ID NO: 86 - DNA, 6151 bp, synthetic construct
  misc_feature 1..6151: note = Plasmid pCEV-G4-Ph
  source 1..6151: mol_type = other DNA
ggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagttttttgattaaaattaaaaaaactttttgtttttgtgtttattctttgttcttagaaaagacaagttgagcttgtttgttcttgatgttttattattttacaatagctgcaaatgaagaatagattcgaacattgtgaagtattggcatatatcgtctctatttatactttttttttttcagttctagtatattttgtattttcctccttttcattctttcagttgccaataagttacaggggatctcgaaagatggtggggatttttccttgaaagacgactttttgccatctaatttttccttgttgcctctgaaaattatccagcagaagcaaatgtaaaagatgaacctcagaagaacacgcaggggcccgaaattgttcctacgagaagtagtgggtcataaaaagtttattccctggaaaaaaaattttgcgttgcctttctggagaattttttcgaattagcgtgctgccactgcatgcatttctgagaagtgtgggcattcttccaccagttgttcctcctaaaaaaaaaaagatttcctaccccgcacaaattcctgcatacccctcatttccacggggccggccgcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaggatccgtaatacgactcactatagggcccgggcgtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatggtagacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtatgaccgaccaagcgacgcccaacctgccatcacgagatttcgatcccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcgacccggacgggacgctcctgcgcctgatacagaacgaattgcttgcaggcatctcatgatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccacta

SEQ ID NO: 87 - DNA, 6861 bp, synthetic construct
  misc_feature 1..6861: note = Plasmid pBIS-GALkFLP-URA3
  source 1..6861: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggatatgtatatggtggtaatgccatgtaatatgattattaaacttctttgcgtccatccaaaaaaaaagtaagaatttttgggtcgacatgccacaatttggtatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctctcattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 88 - DNA, 6857 bp, synthetic construct
  misc_feature 1..6857: note = Plasmid pCEV-URA3-TEF-AT-yABAB-cMyc
  source 1..6857: mol_type = other DNA
agcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccccggccgcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaggatccatgagatttccttcaatttttactgctgttttattcgcagcatcctccgcattagctatgcaagtacaattggttgaaaccggtggtggtttagttcaaccaggtggtagtttgagattatcttgtgctgcatcaggttttacattggattattcttcaataggttggttcagacaagctcctggtaaagaaagagaaggtgtttcttgcatatccagttctggtgactcaactaaatatgctgactccgttaagggtagattcactacttcaagagataacgctaaaaatacagtctacttgcaaatgaactcattaaagccagatgacacagcagtctattactgtgccgcttttagagccaccatgtgcggtgtattcccattgtctccttacggtaaagatgactggggtaaaggtactttagttactgtctcatccggtggtggtggttccggtggtggtggtagtggtggtggtggttctcaagttcaattagtagaatccggtggtggtttagtccaacctggtggtagtttaagattatcctgcgaagcaagtggttttacattagattattacggtatcggttggtttagacaaccacctggtaaagaaagagaagctgtctcttatatttccgctagtgcaagaactatattgtacgcagattctgtaaagggtagattcacaatttcaagagacaatgccaagaacgctgtttatttgcaaatgaactctttgaagagagaagacaccgcagtttattactgtgccagaagaagattttctgcttcttcagtcaacagatggttagcagacgattatgatgtttggggtagaggtacacaagtcgccgtaagttctggtggtggttccggtggtggtagtggtggtggttctggtggtggttcacaattgcaattagtagaaactggtggtggtttggttcaaccaggtggttccttgagattaagttgtgctgcatctggttttactttctctgattacgttatgacatgggtcagacaagctccaggtaaaggtcctgaatggatcgctacaattaataccgacggttccacaatgagagatgacagtaccaagggtagattcactatttcaagagataacgctaagaacacattgtacttacaaatgacctctttgaaaccagaagacaccgcattatattactgtgccagaggtagagtcatatccgccagtgctatcagaggtgcagtaagaggtcctggtactcaagttacagtctcttcaggtggcggcggtagtggcggcggcggttctggcggtggtggttcacaagtccaattggtagaatctggtggtggtttagttcaaactggtggttcattgagattatcctgcgcttccagtggttccattgcaggtttcgaaactgttacatggtcaagacaagctccaggtaaatctttgcaatgggtcgcctcaatgaccaagactaacaacgaaatctattctgattcagttaagggtagattcattatttcaagagataatgctaaaaacaccgtttatttgcaaatgaactcattgaagccagaagatactggtgtttacttctgcaagggtcctgaattaagaggtcaaggtattcaagtaacagtttcttcagtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatggtagacaacccttaatataacttcgtataatgtatgctatacgaagttattaggtctagagatct

SEQ ID NO: 89 - DNA, 5199 bp, synthetic construct
  misc_feature 1..5199: note = Plasmid pCEV-URA3-TEF-cMyc
  source 1..5199: mol_type = other DNA
agcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccagcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagatagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaaaagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgttgtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccccggccgcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaggatctcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatggtagacaacccttaatataacttcgtataatgtatgctatacgaagttattaggtctagagatct

SEQ ID NO: 90 - DNA, 7208 bp, synthetic construct
  misc_feature 1..7208: note = Plasmid pCEV-G4-Km-TEF-AT-yABAB hAA6T83N-tagless
  source 1..7208: mol_type = other DNA
cacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaggatccatgagatttccttcaatttttactgctgttttattcgcagcatcctccgcattagctatgcaagtacaattggttgaaaccggtggtggtttagttcaaccaggtggtagtttgagattatcttgtgctgcatcaggttttacattggattattcttcaataggttggttcagacaagctcctggtaaagaaagagaaggtgtttcttgcatatccagttctggtgactcaactaaatatgctgactccgttaagggtagattcactacttcaagagataacgctaaaaatacagtctacttgcaaatgaactcattaaagccagatgacacagcagtctattactgtgccgcttttagagccaccatgtgcggtgtattcccattgtctccttacggtaaagatgactggggtaaaggtactttagttactgtctcatccggtggtggtggttccggtggtggtggtagtggtggtggtggttctcaagttcaattagtagaatccggtggtggtttagtccaacctggtggtagtttaagattatcctgcgaagcaagtggttttacattagattattacggtatcggttggtttagacaaccacctggtaaagaaagagaagctgtctcttatatttccgctagtgcaagaactatattgtacgcagattctgtaaagggtagattcacaatttcaagagacaatgccaagaacgctgtttatttgcaaatgaactctttgaagagagaagacaccgcagtttattactgtgccagaagaagattttctgcttcttcagtcaacagatggttagcagacgattatgatgtttggggtagaggtacacaagtcgccgtaagttctggtggtggttccggtggtggtagtggtggtggttctggtggtggttcacagctgcagctggtggagaccgggggaggcttagttcagcctggggggtccctgagactctcctgtgcagcctctggattcaccttcagtgactacgtgatgacctgggtccgccaagctccagggaaggggcctgagtggatcgcaactattaatactgatgggagcacaatgcgcgacgactccacaaagggccggttcaccatctccagagacaacgccaagaacactctgtatctgcaaatgaacagtctgaaacccgaggacactgctctgtattactgtgcaagaggccgggtgatctctgcttccgctatcagaggcgcagtcagaggccctggaacccaggtcaccgtctcgagcggtggcggcggtagtggcggcggcggttctggcggtggtggttcacaagtccaattggtagaatctggtggtggtttagttcaaactggtggttcattgagattatcctgcgcttccagtggttccattgcaggtttcgaaactgttacatggtcaagacaagctccaggtaaatctttgcaatgggtcgcctcaatgaccaagactaacaacgaaatctattctgattcagttaagggtagattcattatttcaagagataatgctaaaaacaccgtttatttgcaaatgaactcattgaagccagaagatactggtgtttacttctgcaagggtcctgaattaagaggtcaaggtattcaagtaacagtttcttcagtcgacgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatggtagacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccactag

SEQ ID NO: 91 - DNA, 10379 bp, synthetic construct
  misc_feature 1..10379: note = Plasmid pCRI-Sb-delta1
  source 1..10379: mol_type = other DNA
tcgcgcgaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtcgtttctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgtgctcgagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcgggttttagagctatgctgttttgaatggtcccaaaactactagtatattatcatatagttttagagctatgctgttttgaatggtcccaaaactttttttgttttttatgtctgcatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagaggccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttctagatggattataaagatgacgatgacaaacctccaaaaaagaagagaaaggtcgataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctacttataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattacccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgaccctccaaaaaagaagagaaaggtctgagcggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgagtccctcgatatttctcatactagttctagagatctgccaattgaacataacatggtagttacatatactagtaatatggttcggcacacattaaaagtataaaaactatctgaattacgaattacatatattggtcataaaaatcaatcaatcatcgtgtgttttatatgtctcttatctaagtataagaatatccatagttaatattcacttacgctaccttttaacctgtaatcattgtcaacaggatatgttaacgacccacattgataaacgctagtatttctttttcctcttcttattggccggctgtctctatactcccctatagtctgtttcttttcgtttcgattgttttacgtttgaggcctcgtggcgcacatggtacgctgtggtgctcgcggctgggaacgaaactctgggagctgcgattggcaggaaccattcaaaacagcatagcaagttaaaataaggctagtccgttatcaacttgaaaaagtggcaccgagtcggtgctttttttccatatccaacttccaatttaatctttcttttttaattttcacttatttgcgatacagaaagaccctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggctcccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtc

SEQ ID NO: 92 - DNA, 10379 bp, synthetic construct
  misc_feature 1..10379: note = Plasmid pCRI-Sb-delta2
  source 1..10379: mol_type = other DNA
tcgcgcgaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtcgtttctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgtgctcgagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcgggttttagagctatgctgttttgaatggtcccaaaactatggattcctaaatcctcggttttagagctatgctgttttgaatggtcccaaaactttttttgttttttatgtctgcatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagaggccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttctagatggattataaagatgacgatgacaaacctccaaaaaagaagagaaaggtcgataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctacttataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattacccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgaccctccaaaaaagaagagaaaggtctgagcggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgagtccctcgatatttctcatactagttctagagatctgccaattgaacataacatggtagttacatatactagtaatatggttcggcacacattaaaagtataaaaactatctgaattacgaattacatatattggtcataaaaatcaatcaatcatcgtgtgttttatatgtctcttatctaagtataagaatatccatagttaatattcacttacgctaccttttaacctgtaatcattgtcaacaggatatgttaacgacccacattgataaacgctagtatttctttttcctcttcttattggccggctgtctctatactcccctatagtctgtttcttttcgtttcgattgttttacgtttgaggcctcgtggcgcacatggtacgctgtggtgctcgcggctgggaacgaaactctgggagctgcgattggcaggaaccattcaaaacagcatagcaagttaaaataaggctagtccgttatcaacttgaaaaagtggcaccgagtcggtgctttttttccatatccaacttccaatttaatctttcttttttaattttcacttatttgcgatacagaaagaccctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcggctcccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtc

SEQ ID NO: 93 - DNA, 126 bp, Saccharomyces boulardii
  source 1..126: mol_type = other DNA
tgttggaataaaaatcaactatcatctactaactagtatttacgttactagtatattatcatatacggtgttagaagatgacgcaaatgatgagaaatagtcatctaaattagtggaagctgaaac

SEQ ID NO: 94 - DNA, 105 bp, Saccharomyces boulardii
  source 1..105: mol_type = other DNA
aatatttatagaattgtgtagaattgcagattcccttttatggattcctaaatcctcgaggagaacttctagtatatctacatacctaatattatagccttaatc

SEQ ID NO: 95 - DNA, 6919 bp, synthetic construct
  misc_feature 1..6919: note = Plasmid pPL5071_TEF1-Cre_URA3
  misc_feature 2382: note = n is a, c, g, or t
  source 1..6919: mol_type = other DNA
agaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgaggtcgacaattccaaccttacccaagagttcgccaaactcagacatcactttangcaaaaccgcgccgtgcttcttcctcggtggcattcatcacgaaatgttcagcactacgcatacttttgacaggaaacgcaacggatattgagtcaatatcaggcattctatcgctcagctttacagtgacaatgacggctggcgactgaatattagtgcttacagacagcactacatattttccgtcgatgttgaaatcctttctcatatgtcaccataaatatcaaataattatagcaatcatttacgcgttaatggctaatcgccatcttccagcaggcgcaccattgcccctgtttcactatccagggtacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatttaacactcagataatggttttaagtaaagtgtacaggatcggctctgcccctcgacggtatcgataagcttgatatcgaattcctgcagcccgggggatccactagtttttgtaattaaaacttagattagattgctatgctttctttccaatgagcaagaagtaaaaaaagttgtaatagaacaggaaaaatgaagctgaaacttgagaaattgaagaccgtttgttaactcaaatatcaatgggaggtcgtcgaaagagaacaaaatcgaaaaaaaagttttcaagagaaagaaacgtgataaaaatttttattgccttctccgacgaagaaaaagggacgaggcggtctctttttccttttccaaacctttagtacgggtaattaacggcaccctagaggaaggaggagggggaatttagtatgctgtgcttgggtgttttgaagtggtacggcggtgcgcggagtccgagaaaatctggaagagtaaaaaaggagtagagacattttgaagctatccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtccta

SEQ ID NO: 96 - protein, 89 aa, Saccharomyces cerevisiae
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 97 - protein, 61 aa, Saccharomyces cerevisiae
MRFPSIFTAVLFAASSALAAPVNTTTEDELEGDFDVAVLPFSASIAAKEEGVSLEKREAEA

SEQ ID NO: 98 - protein, 57 aa, Saccharomyces cerevisiae
MRFPSIFTAVLFAASSALAAPVNTTTEDELEGDFDVAVLPFSASIAAKEEGVSLEKR

SEQ ID NO: 99 - protein, 19 aa, Saccharomyces cerevisiae
MRFPSIFTAVLFAASSALA

SEQ ID NO: 100 - protein, 20 aa, Aspergillus niger
MVAWWSLFLYGLQVAAPALA

SEQ ID NO: 101 - protein, 18 aa, Aspergillus awamorii
MSFRSLLALSGLVCSGLA

SEQ ID NO: 102 - protein, 16 aa, Kluyveromyces marxianus
MKLAYSLLLPLAGVSA

SEQ ID NO: 103 - protein, 19 aa, Saccharomyces cerevisiae
MLLQAFLFLLAGFAAKISA

SEQ ID NO: 104 - protein, 26 aa, Saccharomyces cerevisiae
MTKPTQVLVRSVSILFFITLLHLVVA

SEQ ID NO: 105 - protein, 26 aa, Gallus gallus
MLGKNDPMCLVLVLLGLTALLGICQG

SEQ ID NO: 106 - protein, 18 aa, Homo sapiens
MKWVTFISLLFLFSSAYS

SEQ ID NO: 107 - protein, 566 aa, synthetic construct
  REGION 1..566: note = ABAB binding protein expressed from plasmid
MRFPSIFTAVLFAASSALAMQVQLVETGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSGGGGSGGGGSGGGGSQVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSGGGSGGGSGGGSGGGSQLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSGGGGSGGGGSGGGGSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSVDMEQKLISEEDLE

SEQ ID NO: 108 - DNA, 1701 bp, synthetic construct
  misc_feature 1..1701: note = Polynucleotide sequence encoding ABAB binding protein expressed from plasmid
  source 1..1701: mol_type = other DNA
atgagatttccttcaatttttactgctgttttattcgcagcatcctccgcattagctatgcaagtacaattggttgaaaccggtggtggtttagttcaaccaggtggtagtttgagattatcttgtgctgcatcaggttttacattggattattcttcaataggttggttcagacaagctcctggtaaagaaagagaaggtgtttcttgcatatccagttctggtgactcaactaaatatgctgactccgttaagggtagattcactacttcaagagataacgctaaaaatacagtctacttgcaaatgaactcattaaagccagatgacacagcagtctattactgtgccgcttttagagccaccatgtgcggtgtattcccattgtctccttacggtaaagatgactggggtaaaggtactttagttactgtctcatccggtggtggtggttccggtggtggtggtagtggtggtggtggttctcaagttcaattagtagaatccggtggtggtttagtccaacctggtggtagtttaagattatcctgcgaagcaagtggttttacattagattattacggtatcggttggtttagacaaccacctggtaaagaaagagaagctgtctcttatatttccgctagtgcaagaactatattgtacgcagattctgtaaagggtagattcacaatttcaagagacaatgccaagaacgctgtttatttgcaaatgaactctttgaagagagaagacaccgcagtttattactgtgccagaagaagattttctgcttcttcagtcaacagatggttagcagacgattatgatgtttggggtagaggtacacaagtcgccgtaagttctggtggtggttccggtggtggtagtggtggtggttctggtggtggttcacaattgcaattagtagaaactggtggtggtttggttcaaccaggtggttccttgagattaagttgtgctgcatctggttttactttctctgattacgttatgacatgggtcagacaagctccaggtaaaggtcctgaatggatcgctacaattaataccgacggttccacaatgagagatgacagtaccaagggtagattcactatttcaagagataacgctaagaacacattgtacttacaaatgacctctttgaaaccagaagacaccgcattatattactgtgccagaggtagagtcatatccgccagtgctatcagaggtgcagtaagaggtcctggtactcaagttacagtctcttcaggtggcggcggtagtggcggcggcggttctggcggtggtggttcacaagtccaattggtagaatctggtggtggtttagttcaaactggtggttcattgagattatcctgcgcttccagtggttccattgcaggtttcgaaactgttacatggtcaagacaagctccaggtaaatctttgcaatgggtcgcctcaatgaccaagactaacaacgaaatctattctgattcagttaagggtagattcattatttcaagagataatgctaaaaacaccgtttatttgcaaatgaactcattgaagccagaagatactggtgtttacttctgcaagggtcctgaattaagaggtcaaggtattcaagtaacagtttcttcagtcgacatggaacagaagttgatttccgaagaagacctcgagtaa

SEQ ID NO: 109 - protein, 557 aa, synthetic construct
  REGION 1..557: note = ABAB binding protein expressed from chromosomal integration
MRFPSIFTAVLFAASSALAMQVQLVETGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSGGGGSGGGGSGGGGSQVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSGGGSGGGSGGGSGGGSQLQLVETGGGLVQPGGSLRLSCAASGFTFSDYVMTWVRQAPGKGPEWIATINTDGSTMRDDSTKGRFTISRDNAKNTLYLQMNSLKPEDTALYYCARGRVISASAIRGAVRGPGTQVTVSSGGGGSGGGGSGGGGSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSVDAAS

SEQ ID NO: 110 - DNA, 1674 bp, synthetic construct
  misc_feature 1..1674: note = Polynucleotide sequence encoding ABAB binding protein expressed from chromosomal integration
  source 1..1674: mol_type = other DNA
atgagatttccttcaatttttactgctgttttattcgcagcatcctccgcattagctatgcaagtacaattggttgaaaccggtggtggtttagttcaaccaggtggtagtttgagattatcttgtgctgcatcaggttttacattggattattcttcaataggttggttcagacaagctcctggtaaagaaagagaaggtgtttcttgcatatccagttctggtgactcaactaaatatgctgactccgttaagggtagattcactacttcaagagataacgctaaaaatacagtctacttgcaaatgaactcattaaagccagatgacacagcagtctattactgtgccgcttttagagccaccatgtgcggtgtattcccattgtctccttacggtaaagatgactggggtaaaggtactttagttactgtctcatccggtggtggtggttccggtggtggtggtagtggtggtggtggttctcaagttcaattagtagaatccggtggtggtttagtccaacctggtggtagtttaagattatcctgcgaagcaagtggttttacattagattattacggtatcggttggtttagacaaccacctggtaaagaaagagaagctgtctcttatatttccgctagtgcaagaactatattgtacgcagattctgtaaagggtagattcacaatttcaagagacaatgccaagaacgctgtttatttgcaaatgaactctttgaagagagaagacaccgcagtttattactgtgccagaagaagattttctgcttcttcagtcaacagatggttagcagacgattatgatgtttggggtagaggtacacaagtcgccgtaagttctggtggtggttccggtggtggtagtggtggtggttctggtggtggttcacagctgcagctggtggagaccgggggaggcttagttcagcctggggggtccctgagactctcctgtgcagcctctggattcaccttcagtgactacgtgatgacctgggtccgccaagctccagggaaggggcctgagtggatcgcaactattaatactgatgggagcacaatgcgcgacgactccacaaagggccggttcaccatctccagagacaacgccaagaacactctgtatctgcaaatgaacagtctgaaacccgaggacactgctctgtattactgtgcaagaggccgggtgatctctgcttccgctatcagaggcgcagtcagaggccctggaacccaggtcaccgtctcgagcggtggcggcggtagtggcggcggcggttctggcggtggtggttcacaagtccaattggtagaatctggtggtggtttagttcaaactggtggttcattgagattatcctgcgcttccagtggttccattgcaggtttcgaaactgttacatggtcaagacaagctccaggtaaatctttgcaatgggtcgcctcaatgaccaagactaacaacgaaatctattctgattcagttaagggtagattcattatttcaagagataatgctaaaaacaccgtttatttgcaaatgaactcattgaagccagaagatactggtgtttacttctgcaagggtcctgaattaagaggtcaaggtattcaagtaacagtttcttcagtcgacgcggctagctaa

SEQ ID NO: 111 - protein, 549 aa, synthetic construct
  REGION 1..549: note = ABA binding agent
QVQLVETGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSEPKTPKPQPTSGGGGSGGGGSGGGGSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSEPKTPKPQTSAIAGGGGSGGGGSGGGGSLQAMAAASQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSGGGGSGGGGSGGGGSWAAQLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSS

SEQ ID NO: 112 - protein, 15 aa, synthetic construct
  REGION 1..15: note = D7 tag
APTKAKRRVVQREKT

SEQ ID NO: 113 - protein, 553 aa, synthetic construct
  REGION 1..553: note = His-hABAB-D7 binding agent
HHHHHHQVQLVETGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSGGGGSGGGGSGGGGSQVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSGGGSGGGSGGGSGGGSQLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSGGGGSGGGGSGGGGSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSAPTKAKRRVVQREKT

SEQ ID NO: 114 - DNA, 1665 bp, synthetic construct
  misc_feature 1..1665: note = Polynucleotide sequence encoding His-hABAB-D7 binding agent
  source 1..1665: mol_type = other DNA
catcatcatcatcatcatcaggtacagctggtggagacggggggagggctggtacaaccaggcgggtcactgaggctttcctgtgccgcatctgggttcacactggattattcgtccatagggtggtttcggcaggctcctggcaaagagcgtgagggggtctcatgtattagtagtagtggtgatagcacaaagtacgccgattccgtaaagggccggtttacaacctccagggataatgctaagaacaccgtatatctccagatgaactctctgaagcccgacgatacggccgtatattactgtgcggctttcagggcgactatgtgcggcgtgttccctctgagcccttacggcaaggacgactggggcaaggggaccctggtgaccgtatcctcaggcggtggagggtctggtgggggaggctcagggggtggaggcagccaggtgcaactggttgaatctgggggaggcttggtacaacctgggggatccctgagactctcttgcgaggcctccggattcaccttggactactatggcatcggctggttccgccagcccccagggaaggagcgggaggccgtttcatacattagtgccagtgcccggaccatactgtacgcagactctgtgaagggacgctttaccatctctagggacaatgccaaaaatgctgtgtacctgcaaatgaacagcctcaagcgggaggataccgcagtgtactactgcgcgagacggcgcttctccgcttctagcgtgaatagatggctggccgacgactacgacgtgtggggacggggcacacaggtggctgtgtcttccggtggcggaagcggagggggcagcgggggtgggagcggtgggggcagccaactgcagctggtagagacagggggcggcttagttcagcctggagggtctctcagactgtcatgcgctgcctctggctttaccttcagtgactacgtgatgacatgggtccgccaagctccagggaaggggcctgagtggatcgctactattaatacagatggcagcacaatgcgggacgactccacaaaggggcggttcaccatttccagagacaacgccaagaatactctgtaccttcagatgaccagtctgaaacccgaggacactgctctgtactattgtgcaagaggccgggtgatctctgcttccgctatcagaggcgcagtaaggggccctggaacacaggtaaccgtttcatccgggggaggcggttcaggcggtgggggatctggcgggggtggatcccaagttcagctggtcgaatccgggggcggactggtccagacagggggctccctgaggctctcctgtgcatcttccggaagcatcgccggcttcgagaccgtgacctggtctcgccaggctcccgggaagtctctgcagtgggtcgcttccatgactaagactaacaacgagatctactctgactcagtgaaaggccgcttcatcatttctagagataacgctaaaaacacagtgtatctgcagatgaatagtctcaaacctgaagacacaggcgtgtatttctgtaagggtcctgagctgaggggccagggcatccaggtaacagtctcgagtgctcctacaaaagccaaacggagagtggtccagagagagaagacctaataa

SEQ ID NO: 115 - DNA, 7257 bp, synthetic construct
  misc_feature 1..7257: note = Plasmid pCEV-G4-Km-TEF-AT-RSyABAB
  source 1..7257: mol_type = other DNA
cacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaggatccatgagatttccttcaatttttactgctgttttattcgcagcatcctccgcattagctatgcaagtacaattggttgaaaccggtggtggtttagttcaaccaggtggtagtttgagattatcttgtgctgcatcaggttttacattggattattcttcaataggttggttcagacaagctcctggtaaagaaagagaaggtgtttcttgcatatccagttctggtgactcaactaaatatgctgactccgttaagggtagattcactacttcaagagataacgctaaaaatacagtctacttgcaaatgaactcattaaagccagatgacacagcagtctattactgtgccgcttttagagccaccatgtgcggtgtattcccattgtctccttacggtaaagatgactggggtaaaggtactttagttactgtctcatccggtggtggtggttccggtggtggtggtagtggtggtggtggttctcaagttcaattagtagaatccggtggtggtttagtccaacctggtggtagtttaagattatcctgcgaagcaagtggttttacattagattattacggtatcggttggtttagacaaccacctggtaaagaaagagaagctgtctcttatatttccgctagtgcaagaactatattgtacgcagattctgtaaagggtagattcacaatttcaagagacaatgccaagaacgctgtttatttgcaaatgaactctttgaagagagaagacaccgcagtttattactgtgccagaagaagattttctgcttcttcagtcaacagatggttagcagacgattatgatgtttggggtagaggtacacaagtcgccgtaagttctggtggtggttccggtggtggtagtggtggtggttctggtggtggttcacagctgcagctggtggagaccgggggaggcttagttcagcctggggggtccctgagactctcctgtgcagcctctggattcaccttcagtgactacgtgatgacctgggtccgccaagctccagggaaggggcctgagtggatcgcaactattaatactgatgggagcacaatgcgcgacgactccacaaagggccggttcaccatctccagagacaacgccaagaacactctgtatctgcaaatgaacagtctgaaacccgaggacactgctctgtattactgtgcaagaggccgggtgatctctgcttccgctatcagaggcgcagtcagaggccctggaacccaggtcaccgtctcgagcggtggcggcggtagtggcggcggcggttctggcggtggtggttcacaagtccaattggtagaatctggtggtggtttagttcaaactggtggttcattgagattatcctgcgcttccagtggttccattgcaggtttcgaaactgttacatggtcaagacaagctccaggtaaatctttgcaatgggtcgcctcaatgaccaagactaacaacgaaatctattctgattcagttaagggtagattcattatttcaagagataatgctaaaaacaccgtttatttgcaaatgaactcattgaagccagaagatactggtgtttacttctgcaagggtcctgaattaagaggtcaaggtattcaagtaacagtttcttcagtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatggtagacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccactag

SEQ ID NO: 116 - DNA, 8202 bp, synthetic construct
  misc_feature 1..8202: note = Plasmid pCEV-G4-Km-TEF-AT-yABAB AA6T83N
  source 1..8202: mol_type = other DNA
ggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagttttttgattaaaattaaaaaaactttttgtttttgtgtttattctttgttcttagaaaagacaagttgagcttgtttgttcttgatgttttattattttacaatagctgcaaatgaagaatagattcgaacattgtgaagtattggcatatatcgtctctatttatactttttttttttcagttctagtatattttgtattttcctccttttcattctttcagttgccaataagttacaggggatctcgaaagatggtggggatttttccttgaaagacgactttttgccatctaatttttccttgttgcctctgaaaattatccagcagaagcaaatgtaaaagatgaacctcagaagaacacgcaggggcccgaaattgttcctacgagaagtagtgggtcataaaaagtttattccctggaaaaaaaattttgcgttgcctttctggagaattttttcgaattagcgtgctgccactgcatgcatttctgagaagtgtgggcattcttccaccagttgttcctcctaaaaaaaaaaagatttcctaccccgcacaaattcctgcatacccctcatttccacggggccggccgcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaggatccatgagatttccttcaatttttactgctgttttattcgcagcatcctccgcattagctatgcaagtacaattggttgaaaccggtggtggtttagttcaaccaggtggtagtttgagattatcttgtgctgcatcaggttttacattggattattcttcaataggttggttcagacaagctcctggtaaagaaagagaaggtgtttcttgcatatccagttctggtgactcaactaaatatgctgactccgttaagggtagattcactacttcaagagataacgctaaaaatacagtctacttgcaaatgaactcattaaagccagatgacacagcagtctattactgtgccgcttttagagccaccatgtgcggtgtattcccattgtctccttacggtaaagatgactggggtaaaggtactttagttactgtctcatccggtggtggtggttccggtggtggtggtagtggtggtggtggttctcaagttcaattagtagaatccggtggtggtttagtccaacctggtggtagtttaagattatcctgcgaagcaagtggttttacattagattattacggtatcggttggtttagacaaccacctggtaaagaaagagaagctgtctcttatatttccgctagtgcaagaactatattgtacgcagattctgtaaagggtagattcacaatttcaagagacaatgccaagaacgctgtttatttgcaaatgaactctttgaagagagaagacaccgcagtttattactgtgccagaagaagattttctgcttcttcagtcaacagatggttagcagacgattatgatgtttggggtagaggtacacaagtcgccgtaagttctggtggtggttccggtggtggtagtggtggtggttctggtggtggttcacaattgcaattagtagaaactggtggtggtttggttcaaccaggtggttccttgagattaagttgtgctgcatctggttttactttctctgattacgttatgacatgggtcagacaagctccaggtaaaggtcctgaatggatcgctacaattaataccgacggttccacaatgagagatgacagtaccaagggtagattcactatttcaagagataacgctaagaacacattgtacttacaaatgaactctttgaaaccagaagacaccgcattatattactgtgccagaggtagagtcatatccgccagtgctatcagaggtgcagtaagaggtcctggtactcaagttacagtctcttcaggtggcggcggtagtggcggcggcggttctggcggtggtggttcacaagtccaattggtagaatctggtggtggtttagttcaaactggtggttcattgagattatcctgcgcttccagtggttccattgcaggtttcgaaactgttacatggtcaagacaagctccaggtaaatctttgcaatgggtcgcctcaatgaccaagactaacaacgaaatctattctgattcagttaagggtagattcattatttcaagagataatgctaaaaacaccgtttatttgcaaatgaactcattgaagccagaagatactggtgtttacttctgcaagggtcctgaattaagaggtcaaggtattcaagtaacagtttcttcagtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatggtagacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccacta

SEQ ID NO: 117 - protein, 538 aa, synthetic construct
  REGION 1..538: note = AT-yABAB hAA6 T83N binding agent
MQVQLVETGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSGGGGSGGGGSGGGGSQVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSGGGSGGGSGGGSGGGSQLQLVETGGGLVQPGGSLRLSCAASGFTFSDYVMTWVRQAPGKGPEWIATINTDGSTMRDDSTKGRFTISRDNAKNTLYLQMNSLKPEDTALYYCARGRVISASAIRGAVRGPGTQVTVSSGGGGSGGGGSGGGGSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSVDAAS